(19) United States
(12) Patent Application Publication
(10) Pub. No.: US 2019/0350676 A1
(43) Pub. Date: Nov. 21, 2019

(54) MOUTHPIECE, SYSTEM FOR SUPPORTING PRODUCTION OF MOUTHPIECE, MINI-SPLINT, AND METHOD FOR PRODUCING MOUTHPIECE

(71) Applicant: MUSIC CAP TOKYO CORPORATION, Tokyo (JP)

(72) Inventor: Hiroaki ARAI, Tokyo (JP)

(21) Appl. No.: 16/458,742

(22) Filed: Jul. 1, 2019

Related U.S. Application Data
(63) Continuation of application No. PCT/JP2018/038811, filed on Oct. 18, 2018.

(30) Foreign Application Priority Data
May 18, 2018 (JP) 2018-096001
Aug. 10, 2018 (JP) 2018-150927

(51) Int. Cl. A61C 5/00; B29C 43/02; B29C 43/58

(52) U.S. Cl. CPC A61C 5/007; G16H 20/40; B29C 43/58; B29C 43/021

(57) ABSTRACT

A mouthpiece (76) is detachably attached to the maxillary teeth dentition of a patient. A mouthpiece body (78) has an approximately uniform thickness. A mandibular teeth dentition impression (84) in an optimized chewing occlusal jaw position that balances the systemic basal muscle tone of the patient is formed on the occlusal surface of the mouthpiece body (78). When the patient attaches the mouthpiece (76) to the maxillary teeth dentition to achieve a chewing occlusal state or a resting occlusal state, the mandibular teeth dentition is induced to a proper positional relationship that balances a systemic basal muscle tone with respect to the maxillary teeth dentition due to the tongue/jaw position reflex and masseter/jaw position reflex of the patient, caused by attaching the mouthpiece (76), whereby a systemic pain symptom can be resolved or greatly relieved.

### TECHNICAL FIELD

The present invention relates to: a mouthpiece that corrects a mandibular position at a proper position in order to optimize the balance of a systemic basal muscle tone; a system for supporting production of a mouthpiece; a mini-splint; and a method for producing a mouthpiece.

### BACKGROUND ART

Mouthpieces and mini-splints, which are not natural materials but are artificial products, are structures attached to human maxillary teeth dentitions.

Conventionally, favorable reactions have not necessarily been able to be provided from patients in the case of forming teeth thought to be ideal by dentists in occlusal cares.

Conventionally, medical examinations have been performed primarily only with oral models as observation objects. However, it is a fact that the teeth are parts of the skeleton of the face, and the certain deformation of the skeletal system from the cranial bone to the face and the mandibular bone is not rare at all in the experience of neurosurgeons.

Teeth seen to be normal in the case of observing only a dental oral model may be inappropriate for the form of the teeth of a patient having the certain deformation of the skeleton of the face, and cause unidentified complaints.

Unidentified complaints are conditions of which the causative diseases are unknown even in the case of inspection although subjective symptoms such as the chronic pain of each site in the body, various autonomic nervous symptoms, an unremoved feeling of fatigue, and difficulty in sleeping are complained. The unidentified complaints are characterized in that the complaints (chief complaints) from patients are intensive but are subjective and various, and have less objective findings. Since it is impossible to find the basic causes of the unidentified complaints, treatment of the unidentified complaints is also difficult, and it is also difficult to understand the unidentified complaints by those around the patients.

### RELATED ART DOCUMENTS

### Patent Documents

Patent Document 1: Japanese Patent Laid-Open No. 2017-080027

Patent Document 2: Japanese Patent Laid-Open No. 2018-061639

### Non Patent Documents

Non Patent Document 1: “Jaw Position Disorder Vol. 1”, written by Hiroaki Arai and published by MUSIC CAP TOKYO on Jul. 17, 2015

Non Patent Document 2: “Jaw Position Disorder Vol. 2”, written by Hiroaki Arai and published by MUSIC CAP TOKYO on Jul. 23, 2015

Non Patent Document 3: “Jaw Position Disorder Vol. 3”, written by Hiroaki Arai and published by MUSIC CAP TOKYO on Jul. 23, 2015

### DISCLOSURE OF INVENTION

### Problems to be Solved by the Invention

There are a few dentists proposing the improvement of such unidentified complaints. However, there are many patients who not only experience no improvement of conditions even if receiving treatment in any places for dental treatment but also develop the conditions worsened by further receiving the treatment, and who have a sense of fear about dental practice.

Even in such present circumstances, there is no unified view in the field of dental practice, and even guidelines are not currently developed.

The fact has been confirmed that patients with medical disease names include many patients who do not notice that the patients developed the diseases but who develop the diseases due to dental practice. However, many in the field of dental practice currently deny the influence of the jaw position on the whole body.

An objective of the present invention is to clarify the causal relation between the change of mandibular position due to, for example, dental practice, aging, and congenital causes and the corresponding change of the balance of a systemic basal muscle tone, and to provide: a mouthpiece that can relatively inexpensively resolve a systemic pain symptom caused by an unidentified complaint such as fibromyalgia without the invasion of a tooth while demonstrating that the mouthpiece has universality and reproducibility; a system for supporting production of the mouthpiece; a mini-splint; and a method for producing the mouthpiece.

### Solution to Problem

(First Invention: Mouthpiece)

The present invention is to provide a mouthpiece that is detachably attached to the maxillary teeth dentition of a patient,

wherein the teeth mark of a mandibular teeth dentition in an optimized chewing occlusal jaw position that balances the systemic basal muscle tone of the patient is formed on an occlusal surface of a mouthpiece body having an approximately uniform thickness.

(Material of Mouthpiece)

The mouthpiece body includes a soft-type material or a hard-type material.

(Second Invention: System for Supporting Production of Mouthpiece)

In another embodiment of the present invention, a system for supporting production of a mouthpiece that is detachably mounted on the maxillary teeth dentition of a patient, the system including:

jaw model guidance means that guides production of maxillary and mandibular models on which the teeth dentitions of the patient are arranged;

chewing occlusal jaw position guidance means that guides a medical care operation by which a chewing occlusal jaw position optimized for the patient is constructed;

optimum occlusion taking guidance means that guides production of an optimum occlusal impression member on which, in the state of constructing a chewing occlusal jaw position optimized for the patient according to the guidance of the chewing occlusal jaw position guidance means, the occlusal teeth dentitions of the optimized chewing occlusal jaw position are impressed;

occlusal model production guidance means that fixes the optimum occlusal impression member produced according to the guidance of the optimum occlusion taking guidance means to an occludator in the state of sandwiching the optimum occlusal impression member between the teeth dentitions of the maxillary model and the mandibular model to guide an operation of constructing an optimized chewing occlusal model;

mouthpiece production guidance means that guides production of a mouthpiece on which the teeth dentition impression of the maxillary model and the teeth dentition impression of the mandibular model are formed by the occludator in which the chewing occlusal model is constructed, so that the optimized chewing occlusal jaw position is achieved, and which has an approximately uniform thickness; and

mouthpiece fine adjustment guidance means that guides a medical care operation of finely adjusting the mouthpiece so that the systemic basal muscle tone of the patient is balanced to obtain an optimum resting jaw position in the resting jaw position state of the patient on which the mouthpiece produced by the mouthpiece production guidance means is mounted.

(Chewing Occlusal Jaw Position Guidance)

The chewing occlusal jaw position guidance means includes:

mandibular angle position correction guidance means that guides a medical care operation of correcting the lateral position of a mandibular angle at an optimized position using the tongue/jaw position reflex of the patient, based on the hypertonia of the posterior cervical region of the patient, input by a screen operation through a display;

mental region position correction guidance means that guides a medical care operation of correcting the lateral position of the mental region at an optimized position using the tongue/jaw position reflex of the patient, based on the hypertonia of the anterior cervical region of the patient, input by a screen operation through the display;

height difference correction guidance means that guides a medical care operation of correcting a difference between the heights of the mandibular angle and the mental region using the masseter/jaw position reflex of the patient in a state in which corrections according to the guidance of the mandibular angle position correction guidance means and the guidance of the mental region position correction guidance means have been finished; and

anteroposterior position adjustment guidance means that guides a medical care operation of adjusting the anteroposterior position of a mandibular position at an optimized position in a state in which a correction according to the guidance of the height difference correction guidance means has been finished.

(Mandibular Angle Position Correction Guidance)

The mandibular angle position correction guidance means

guides a medical care operation of putting wax on the lingual tooth surface of a mandibular right molar tooth and adjusting the thickness of the wax so that the laterality of the basal muscle tone of the posterior cervical region is eliminated, when the enhancement of the basal muscle tone of the right of the posterior cervical region is input by a screen operation through the display;

guides a medical care operation of putting wax on the lingual tooth surface of a mandibular left molar tooth and adjusting the thickness of the wax so that the laterality of the basal muscle tone of the posterior cervical region is eliminated, when the enhancement of the basal muscle tone of the left of the posterior cervical region is input by a screen operation through the display; and

guides a medical care operation of preventing wax from being put on the lingual tooth surface of a mandibular molar tooth when no enhancement of the basal muscle tone of the posterior cervical region is input by a screen operation through the display and there is no laterality.

(Mental Region Position Correction Guidance)

The mental region position correction guidance means

guides a medical care operation of putting wax on the lingual tooth surface of a mandibular right premolar tooth and adjusting the thickness of the wax so that the laterality of the basal muscle tone of the anterior cervical region is eliminated, when the enhancement of the basal muscle tone of the right of the anterior cervical region is input by a screen operation through the display;

guides a medical care operation of putting wax on the lingual tooth surface of a mandibular left premolar tooth and adjusting the thickness of the wax so that the laterality of the basal muscle tone of the anterior cervical region is eliminated, when the enhancement of the basal muscle tone of the left of the anterior cervical region is input by a screen operation through the display; and

guides a medical care operation of preventing wax from being put on the lingual tooth surface of a mandibular premolar tooth when no enhancement of the basal muscle tone of the anterior cervical region is input by a screen operation through the display and there is no laterality.

(Height Difference Correction Guidance)

The height difference correction guidance means guides a medical care operation of putting wax on the occlusal surfaces of both maxillary first molar teeth to configure a chewing jaw position, measuring a forced expiratory residual volume with a spirometer after induction of a masseter/jaw position reflex, and correcting the elevation angle of the occlusal reference plane of a mandibular teeth dentition with respect to a horizontal reference plane so that the forced expiratory residual volume is not more than a predetermined value.

(Forced Expiratory Residual Volume)

The height difference correction guidance means guides a medical care operation of correcting the elevation angle so that the forced expiratory residual volume is 500 milliliters or less.

(Mini-Splint)

The height difference correction guidance means guides a medical care operation of preparing a mini-splint in which the predetermined, corrected elevation angle of the occlusal reference plane of the mandibular teeth dentition with respect to the horizontal reference plane is set, and mounting the mini-splint on the mandibular teeth dentition to measure the forced expiratory residual volume with the spirometer.

(Selection of Mini-Splint)

The height difference correction guidance means guides a medical care operation of preparing a plurality of kinds of mini-splints of which the elevation angle-corrected angles are different, selecting a mini-splint having a predetermined elevation angle-corrected angle from the mini-splints, and using the mini-splint.

(Anteroposterior Position Adjustment Guidance)

The anteroposterior position adjustment guidance means guides a medical care operation of finely adjusting the anteroposterior position of a chewing occlusal jaw position so that the head position of the patient in rest and relaxation is maintained at a midline position with respect to the visual line of the patient.

(Adjustment of Anteroposterior Positions of Forward Bending Position and Backward Bending Position)

The anteroposterior position adjustment guidance means guides a medical care operation of moving forward a mandibular position when the head position of the patient in the rest and relaxation is a forward bending position and moving backward the mandibular position when the head position is a backward bending position.

(Assistance of Adjustment of Anteroposterior Position)

In a case in which the patient is incapable of single-handedly finely adjusting an anteroposterior position, the anteroposterior position adjustment guidance means

guides a medical care operation of putting wax on the lingual tooth surface of a mandibular anterior teeth portion to adjust the thickness of the wax so that forward movement is assisted when the head position of the patient in the rest and relaxation is a forward bending position, and

guides a medical care operation of putting wax on the lingual tooth surface of a maxillary anterior teeth portion to adjust the thickness of the wax so that backward movement is assisted when the head position of the patient in the rest and relaxation is a backward bending position.

(Mouthpiece Production Guidance)

The mouthpiece production guidance means

guides production of a mouthpiece part on which the maxillary teeth dentition is carved based on the maxillary model, and which has an approximately uniform thickness;

then guides an operation of sandwiching the optimum occlusal impression member between the maxillary model and the mandibular model to fix the maxillary model and the mandibular model to an occludator to reproduce the optimum chewing occlusal jaw position; and

then guides an operation of mounting the mouthpiece part on the maxillary model of the occludator from which the optimum occlusal impression member is removed, and applying the teeth dentition impression of the mandibular model to an occlusal surface of the mouthpiece part based on occlusion with the mandibular model via the mouthpiece part to complete a mouthpiece.

(Guidance of Fine Adjustment of Mouthpiece Corresponding to Adjustment of Position of Mandibular Angle)

The mouthpiece fine adjustment guidance means,

in the state of the resting jaw position of the patient in which the completed mouthpiece is mounted on the maxillary teeth dentition of the patient,

guides a fine adjustment operation of polishing the thickness of the mouthpiece located on the lingual tooth surface of a maxillary right molar tooth to reduce the thickness of the mouthpiece so that the laterality of the basal muscle tone of the posterior cervical region is eliminated when the enhancement of the basal muscle tone of the right of the posterior cervical region is input by a screen operation through the display,

guides a fine adjustment operation of polishing the thickness of the mouthpiece located on the lingual tooth surface of the maxillary left molar tooth to reduce the thickness of the mouthpiece so that the laterality of the basal muscle tone of the posterior cervical region is eliminated when the enhancement of the basal muscle tone of the left of the posterior cervical region is input by a screen operation through the display, and

guides a fine adjustment operation of performing no fine adjustment by polishing of the mouthpiece when no enhancement of the basal muscle tone of the posterior cervical region is input by a screen operation through the display and there is no laterality.

(Guidance of Fine Adjustment of Mouthpiece Corresponding to Adjustment of Position of Mental Region)

The mouthpiece fine adjustment guidance means,

in the state of the resting jaw position of the patient in which the completed mouthpiece is mounted on the maxillary teeth dentition of the patient,

guides a fine adjustment operation of polishing the thickness of the mouthpiece located on the lingual tooth surface of a maxillary right premolar tooth to reduce the thickness of the mouthpiece so that the laterality of the basal muscle tone of the anterior cervical region is eliminated when the enhancement of the basal muscle tone of the right of the anterior cervical region is input by a screen operation through the display,

guides a fine adjustment operation of polishing the thickness of the mouthpiece located on the lingual tooth surface of the maxillary left premolar tooth to reduce the thickness of the mouthpiece so that the laterality of the basal muscle tone of the anterior cervical region is eliminated when the enhancement of the basal muscle tone of the left of the anterior cervical region is input by a screen operation through the display, and

guides a fine adjustment operation of performing no fine adjustment by polishing of the mouthpiece when no enhancement of the basal muscle tone of the anterior cervical region is input by a screen operation through the display and there is no laterality.

(Guidance of Fine Adjustment of Mouthpiece Corresponding to Adjustment of Anteroposterior Position)

The mouthpiece fine adjustment guidance means

in the resting jaw position state of the patient in which the completed mouthpiece is mounted on the maxillary teeth dentition of the patient,

guides a fine adjustment operation of polishing the thickness of the mouthpiece located on the lingual tooth surface of a maxillary anterior teeth portion to reduce the thickness of the mouthpiece so that the forward movement of the mandible is assisted when an input in that the head position of the patient in rest and relaxation is a forward bending position is performed by a screen operation through the display, and

guides a fine adjustment operation of increasing the thickness of the mouthpiece located on the lingual tooth surface of the maxillary anterior teeth portion to increase the thickness of the mouthpiece so that the backward movement of the mandible is assisted when an input in that the head position of the patient in the rest and relaxation is a backward bending position is performed by a screen operation through the display.

(Adjustment of Thickness in Micron Order)

The mouthpiece fine adjustment guidance means guides a fine adjustment operation of changing the thickness of a mouthpiece body in a unit of micron order.

(Tool Guidance)

Tool guidance means that guides a predetermined tool used for a treatment operation for producing the mouthpiece is further included, and, for example, the tool guidance means guides: a posture measurement instrument that measures the posture of a patient; and an electromyograph that measures the enhancement of the basal muscle tones of the posterior and anterior cervical regions of the patient.

(Third Invention: Mini-Splint)

The present invention is to provide a mini-splint used in construction of an optimized chewing occlusal jaw position that balances the systemic basal muscle tone of a patient, the mini-splint including:

a fixation portion that is detachably fixedly mounted between at least a second premolar tooth and a first premolar tooth in the mandibular teeth dentition of the patient; and

an angle-corrected surface that is disposed integrally with the fixation portion and fitted to the occlusal surface of the first molar tooth to set a predetermined elevation angle-corrected angle with respect to the predetermined buccal cusp of the first molar tooth.

(Mini-Splints Having Different Elevation Angles)

A plurality of kinds of mini-splints of which the elevation angle-corrected angles due to the angle-corrected surface are different are prepared, and a mini-splint having a predetermined elevation angle-corrected angle is selected from the mini-splints and fixedly mounted between at least a second premolar tooth and a first molar tooth in the mandibular teeth dentition of the patient through the fixation portion.

(Fourth Invention: Method for Producing Mouthpiece)

The present invention is to provide a method for producing a mouthpiece that is detachably mounted on the maxillary teeth dentition of a patient to configure an optimized chewing occlusal jaw position that balances the systemic basal muscle tone of the patient, the method including:

a maxillary and mandibular models production procedure of producing a maxillary model and a mandibular model on which the teeth dentitions of the patient are arranged;

a mandibular angle position correction procedure of correcting the lateral position of a mandibular angle at an optimized position using the tongue/jaw position reflex of the patient;

a mental region position correction procedure of correcting the lateral position of the mental region at an optimized position using the tongue/jaw position reflex of the patient;

a height difference correction procedure of correcting a difference between the heights of the mandibular angle and the mental region using the masseter/jaw position reflex of the patient in a state in which corrections according to the mandibular angle position correction procedure and the mental region position correction procedure have been finished;

an anteroposterior position adjustment procedure of adjusting the anteroposterior position of a mandibular position to an optimized position in a state in which a correction according to the height difference correction procedure has been finished;

an optimum occlusion taking procedure of producing an optimum occlusal impression member on which the occlusal teeth dentitions of the optimized chewing occlusal jaw position are impressed in a state in which a chewing occlusal jaw position optimized for the patient is constructed by the anteroposterior position adjustment procedure;

an occlusal model production procedure of constructing an optimized chewing occlusal model by fixing the optimum occlusal impression member produced in the optimum occlusion taking procedure to an occludator in a state in which the optimum occlusal impression member is sandwiched between the teeth dentitions of the maxillary model and the mandibular model;

a mouthpiece production procedure of producing a mouthpiece on which the teeth dentition impression of a maxillary tooth shape and the teeth dentition impression of a mandibular tooth shape are formed, so that an optimized chewing occlusal jaw position is achieved, by the occludator in which the chewing occlusal model is constructed, and which has an approximately uniform thickness; and

a mouthpiece fine adjustment procedure of performing fine adjustment so that the systemic basal muscle tone of the patient is balanced to obtain an optimum resting jaw position in the resting jaw position state of the patient on which the mouthpiece produced in the mouthpiece production procedure is mounted.

(Details of Mandibular Angle Position Correction Procedure)

In the mandibular angle position correction procedure,

in the occurrence of the enhancement of a basal muscle tone in the right of the posterior cervical region, wax is put on the lingual tooth surface of a mandibular right molar tooth to adjust the thickness of the wax so that the laterality of the basal muscle tone of the posterior cervical region is eliminated;

in the occurrence of the enhancement of a basal muscle tone in the left of the posterior cervical region, wax is put on the lingual tooth surface of a mandibular left molar tooth to adjust the thickness of the wax so that the laterality of the basal muscle tone of the posterior cervical region is eliminated; and

in the absence of the laterality of the basal muscle tone of the posterior cervical region, the wax is not put on the lingual tooth surface of a mandibular molar tooth.

(Details of Mental Region Position Correction Procedure)

In the mental region position correction procedure,

in the occurrence of the enhancement of a basal muscle tone in the right of the anterior cervical region, wax is put on the lingual tooth surface of a mandibular right premolar tooth to adjust the thickness of the wax so that the laterality of the basal muscle tone of the posterior cervical region is eliminated;

in the occurrence of the enhancement of a basal muscle tone in the left of the anterior cervical region, wax is put on the lingual tooth surface of a mandibular left premolar tooth to adjust the thickness of the wax so that the laterality of the basal muscle tone of the posterior cervical region is eliminated; and

in the absence of the laterality of the basal muscle tone of the anterior cervical region, the wax is not put on the lingual tooth surface of a mandibular premolar tooth.

(Height Difference Correction Procedure)

In the height difference correction procedure, wax is put on the occlusal surfaces of both maxillary first molar teeth to configure a chewing jaw position, a forced expiratory residual volume is measured with a spirometer after induction of a masseter/jaw position reflex, and the elevation angle of the occlusal reference plane of a mandibular teeth dentition with respect to a horizontal reference plane is corrected so that the forced expiratory residual volume is not more than a predetermined value.

(Upper Limit Value of Forced Expiratory Residual Volume)

The elevation angle is corrected so that the forced expiratory residual volume is 500 milliliters or less.

(Mini-Splint)

A mini-splint in which the predetermined, corrected elevation angle of the occlusal reference plane of the mandibular teeth dentition with respect to the horizontal reference plane is set is prepared, and the mini-splint is mounted on the mandibular teeth dentition to measure the forced expiratory residual volume with the spirometer.

(Details of Mini-Splint)

The mini-splint includes:

a fixation portion that is detachably fixedly mounted on at least a second premolar tooth of the mandible; and

an angle-corrected surface that is formed on the occlusal surface of the first molar tooth to set a predetermined elevation angle-corrected angle with respect to the predetermined buccal cusp of the first molar tooth.

(Mini-Splints Having Different Elevation Angles)

In the mini-splint, a plurality of kinds of mini-splints of which the elevation angle-corrected angles are different are prepared, and a mini-splint having a predetermined elevation angle-corrected angle is selected from the mini-splints and used.

(Anteroposterior Position Adjustment Procedure)

In the anteroposterior position adjustment procedure,

the anteroposterior position of a chewing occlusal jaw position is finely adjusted so that the head position of the patient in rest and relaxation is maintained at a midline position with respect to the visual line of the patient.

(Details of Anteroposterior Position Adjustment Procedure)

In the anteroposterior position adjustment procedure, a mandibular position is moved forward when the head position of the patient in the rest and relaxation is a forward bending position, and the mandibular position is moved backward when the head position is a backward bending position.

(Assistance of Forward and Backward Movement by Putting Wax)

In the anteroposterior position adjustment procedure, in a case inwhich the patient is incapable of single-handedly finely adjusting an anteroposterior position,

wax is put on the lingual tooth surface of a mandibular anterior teeth portion to adjust the thickness of the wax so that forward movement is assisted when the head position of the patient in the rest and relaxation is a forward bending position, and

wax is put on the lingual tooth surface of a maxillary anterior teeth portion to adjust the thickness of the wax so that backward movement is assisted when the head position is a backward bending position.

(Production of Mouthpiece)

The mouthpiece production procedure includes:

a procedure of producing a mouthpiece part on which a maxillary teeth dentition is carved based on the produced maxillary model of a patient, and which has an approximately uniform thickness;

a procedure of, based on a produced optimum occlusal impression member which balances the systemic basal muscle tone of the patient, and on which the impressions of a maxillary teeth dentition and a mandibular teeth dentition in an optimum chewing occlusal jaw position are carved, fixing the produced maxillary and mandibular models of the patient to an occludator to reproduce the optimum chewing occlusal jaw position;

a procedure of mounting the mouthpiece part on the maxillary model fixed to the occludator and applying the teeth dentition impression of the mandibular model to an occlusal surface of the mouthpiece part based on occlusion with the mandibular model via the mouthpiece part to produce a mouthpiece; and

a procedure of finely adjusting the thickness of the mouthpiece located on the lingual tooth surface of the completed mouthpiece so that an optimum resting occlusal jaw position is obtained when the completed mouthpiece is mounted on the patient.

(Fine Adjustment of Mouthpiece Corresponding to Mandibular Angle)

In the mouthpiece fine adjustment procedure, in the state of the resting jaw position of the patient on which the completedmouthpiece is mounted,

the thickness of the mouthpiece located on the lingual tooth surface of a maxillary right molar tooth is polished to reduce the thickness of the mouthpiece so that the laterality of the basal muscle tone of the posterior cervical region is eliminated in the occurrence of the enhancement of the basal muscle tone of the right of the posterior cervical region,

the thickness of the mouthpiece located on the lingual tooth surface of the maxillary left molar tooth is polished to reduce the thickness of the mouthpiece so that the laterality of the basal muscle tone of the posterior cervical region is eliminated in the occurrence of the enhancement of the basal muscle tone of the left of the posterior cervical region, and

no fine adjustment by polishing of the mouthpiece is performed in the absence of the laterality of the basal muscle tone of the posterior cervical region.

(Fine Adjustment of Mouthpiece Corresponding to Mental Region)

In the mouthpiece fine adjustment procedure, in the state of the resting jaw position of the patient on which the completedmouthpiece is mounted,

the thickness of the mouthpiece located on the lingual tooth surface of a maxillary right premolar tooth is polished to reduce the thickness of the mouthpiece so that the laterality of the basal muscle tone of the anterior cervical region is eliminated in the occurrence of the enhancement of the basal muscle tone of the right of the anterior cervical region,

the thickness of the mouthpiece located on the lingual tooth surface of the maxillary left premolar tooth is polished to reduce the thickness of the mouthpiece so that the laterality of the basal muscle tone of the anterior cervical region is eliminated in the occurrence of the enhancement of the basal muscle tone of the left of the anterior cervical region, and

no fine adjustment by polishing of the mouthpiece is performed in the absence of the laterality of the basal muscle tone of the anterior cervical region.

(Assistance of Forward and Backward Movement by Putting Wax)

In the mouthpiece fine adjustment procedure, in the resting jaw position state of the patient on which the completed mouthpiece is mounted,

the thickness of the mouthpiece located on the lingual tooth surface of a maxillary anterior teeth portion is polished to reduce the thickness of the mouthpiece so that the forward movement of the mandible is assisted when the head position of the patient in rest and relaxation is a forward bending position, and

the thickness of the mouthpiece located on the lingual tooth surface of the maxillary anterior teeth portion is increased to increase the thickness of the mouthpiece so that the backward movement of the mandible is assisted when the head position of the patient in the rest and relaxation is a backward bending position.

(Fine Adjustment of Thickness in Micron Order)

The thickness of the mouthpiece is changed (decreased or increased) in a unit of micron order.

(Soft Type)

In the mouthpiece production procedure, the mouthpiece including a soft-type material is produced.

(Hard Type)

In the mouthpiece production procedure, the mouthpiece including a hard-type material is produced.

### Advantageous Effects of Invention

(First Invention: Effects of Mouthpiece)

In the present invention, the same effects as the effects of a system for supporting production of a mouthpiece according to the second invention described below can be obtained because the present invention is to provide a mouthpiece that is detachably attached to the maxillary teeth dentition of a patient, wherein the teeth mark of a mandibular teeth dentition in an optimized chewing occlusal jawposition that balances the systemic basal muscle tone of the patient is formed on an occlusal surface of a mouthpiece body having an approximately uniform thickness.

(Effects Due to Material of Mouthpiece)

The mouthpiece body includes a soft-type or hard-type material. Therefore, when first, a soft-type mouthpiece on which the teeth mark of a mandibular teeth dentition in an optimized chewing occlusal jaw position that balances a systemic basal muscle tone is formed is produced and used, and it can be confirmed that a chewing occlusal jaw position and a resting occlusal jaw position are allowed to be adequate using the soft-type mouthpiece, whereby appropriate jaw movement is maintained even in opening and closing mouth movement, a mouthpiece is produced using a hard-type material to which the teeth mark of the mandibular teeth dentition in the optimized chewing occlusal jaw position is applied, and which is finely adjusted, so that a form equivalent to the produced soft-type mouthpiece is achieved as a mouthpiece in a second stage, whereby the wear and degradation of the material are prevented to enable permanent use of the mouthpiece.

(Second Invention: Effects of System for Supporting Production of Mouthpiece)

In the present invention, a system for supporting production of a mouthpiece that is detachably mounted on the maxillary teeth dentition of a patient includes: jaw model guidance means that guides production of maxillary and mandibular models on which the teeth dentitions of the patient are arranged; chewing occlusal jaw position guidance means that guides a medical care operation by which a chewing occlusal jaw position optimized for the patient is constructed; optimum occlusion taking guidance means that guides production of an optimum occlusal impression member on which, in the state of constructing a chewing occlusal jaw position optimized for the patient according to the guidance of the chewing occlusal jaw position guidance means, the occlusal teeth dentitions of the optimized chewing occlusal jaw position are impressed; an occlusal model production guidance procedure that fixes the optimum occlusal impression member produced according to the guidance of the optimum occlusion taking guidance procedures to an occludator in the state of sandwiching the optimum occlusal impression member between the teeth dentitions of the maxillary model and the mandibular model to guide an operation of constructing an optimized chewing occlusal model; mouthpiece production guidance means that guides production of a mouthpiece on which the teeth dentition impression of the maxillary model and the teeth dentition impression of the mandibular model are formed by the occludator in which the chewing occlusal model is constructed, so that the optimized chewing occlusal jaw position is achieved, and which has an approximately uniform thickness; and mouthpiece fine adjustment guidance means that guides a medical care operation of finely adjusting the mouthpiece so that the systemic basal muscle tone of the patient is balanced to obtain an optimum resting jaw position in the resting jaw position state of the patient on which the mouthpiece produced by the mouthpiece production guidance means is mounted. Therefore, the optimized chewing occlusal jaw position that balances the systemic basal muscle tone of the patient is constructed by a medical care operation according to the guidance of the chewing occlusal jaw position guidance means, an optimum occlusal impression member is subsequently produced according to the guidance of an optimum occlusion taking guidance procedure, the mouthpiece on which the teeth dentition impression of a maxillary tooth shape and the teeth dentition impression of a mandibular tooth shape are formed, and which has an approximately uniform thickness so that the optimized chewing occlusal jaw position is achieved is subsequently produced according to the guidance of the mouthpiece production guidance means, and the optimized resting jaw position is further constructed by the fine adjustment of the mouthpiece according to the guidance of the mouthpiece fine adjustment guidance means. When a chewing occlusal state or a resting jaw position state is constructed in a state in which the mouthpiece produced in such a manner is mounted on the maxillary teeth dentition of the patient, the mandibular teeth dentition of the patient occludes with and is positioned at the mandibular teeth dentition impression on the undersurface of the mouthpiece, whereby the chewing occlusal jaw position or the resting jaw position is allowed to be adequate so that the mandible is at a proper position. Thus, the lateral balance of the basal muscle tone of the posterior cervical region and the lateral balance of the basal muscle tone of the anterior cervical region becomes equivalent to each other, and the systemic basal muscle tone is balanced. As a result, a systemic pain symptom caused by an unidentified complaint such as fibromyalgia can be resolved or greatly relieved.

Since it is essential only to mount the mouthpiece on the maxillary teeth dentition, a teeth is not invaded at all, the systemic basal muscle tone is relatively inexpensively balanced, and a systemic pain symptom caused by an unidentified complaint such as fibromyalgia can be instantly resolved or greatly relieved.

(Effects of Chewing Occlusal Jaw Position Guidance)

The chewing occlusal jaw position guidance means includes: mandibular angle position correction guidance means that guides a medical care operation of correcting the lateral position of a mandibular angle at an optimized position using the tongue/jaw position reflex of the patient, based on the hypertonia of the posterior cervical region of the patient, input by a screen operation through a display; mental region position correction guidance means that guides a medical care operation of correcting the lateral position of the mental region at an optimized position using the tongue/jaw position reflex of the patient, based on the hypertonia of the anterior cervical region of the patient, input by a screen operation through the display; height difference correction guidance means that guides a medical care operation of correcting a difference between the heights of the mandibular angle and the mental region using the masseter/jaw position reflex of the patient in a state in which corrections according to the guidance of the mandibular angle position correction guidance means and the guidance of the mental region position correction guidance means have been finished; and anteroposterior position adjustment guidance means that guides a medical care operation of adjusting the anteroposterior position of a mandibular position at an optimized position in a state in which a correction according to the guidance of the height difference correction guidance means has been finished. Therefore, the medical care operations according to the guidances enable the systemic basal muscle tone of the patient to be balanced and an optimized chewing occlusal jaw position to be reliably constructed.

(Effects of Mandibular Angle Position Correction Guidance)

The mandibular angle position correction guidance means guides a medical care operation of putting wax on the lingual tooth surface of a mandibular right molar tooth and adjusting the thickness of the wax so that the laterality of the basal muscle tone of the posterior cervical region is eliminated, when the enhancement of the basal muscle tone of the right of the posterior cervical region is input by a screen operation through the display, guides a medical care operation of putting wax on the lingual tooth surface of a mandibular left molar tooth and adjusting the thickness of the wax so that the laterality of the basal muscle tone of the posterior cervical region is eliminated, when the enhancement of the basal muscle tone of the left of the posterior cervical region is input by a screen operation through the display, and guides a medical care operation of preventing wax from being put on the lingual tooth surface of a mandibular molar tooth when no enhancement of the basal muscle tone of the posterior cervical region is input by a screen operation through the display and there is no laterality. Therefore, a chewing occlusal jaw position which is induced by a tongue/jaw position reflex to the position of the proper mandibular angle that balances the basal muscle tone of the posterior cervical region, and in which the enhancement of the basal muscle tone of the posterior cervical region is eliminated is constructed by primarily applying the wax to the lower tooth surface of the mandibular molar tooth to adjust the thickness to achieve the lateral balance while confirming the balance of the basal muscle tone of the posterior cervical region of the patient according to the guidances.

(Effects of Mental Region Position Correction Guidance)

The mental region position correction guidance means guides a medical care operation of putting wax on the lingual tooth surface of a mandibular right premolar tooth and adjusting the thickness of the wax so that the laterality of the basal muscle tone of the anterior cervical region is eliminated, when the enhancement of the basal muscle tone of the right of the anterior cervical region is input by a screen operation through the display, guides amedical care operation ofputting wax on the lingual tooth surface of a mandibular left premolar tooth and adjusting the thickness of the wax so that the laterality of the basal muscle tone of the anterior cervical region is eliminated, when the enhancement of the basal muscle tone of the left of the anterior cervical region is input by a screen operation through the display, and guides a medical care operation of preventing wax from being put on the lingual tooth surface of a mandibular premolar tooth when no enhancement of the basal muscle tone of the anterior cervical region is input by a screen operation through the display and there is no laterality. Therefore, a chewing occlusal jaw position which is induced by a tongue/jaw position reflex to the position of the mental region that balances the basal muscle tone of the anterior cervical region, and in which the enhancement of the basal muscle tone of the anterior cervical region is eliminated is constructed by primarily applying the wax to the lower tooth surface of the mandibular premolar tooth to adjust the thickness to achieve the lateral balance while confirming the balance of the basal muscle tone of the anterior cervical region of the patient according to the guidances.

(Effects of Height Difference Correction Guidance)

The height difference correction guidance means guides a medical care operation of putting wax on the occlusal surfaces of both maxillary first molar teeth to configure a chewing jaw position, measuring a forced expiratory residual volume with a spirometer after induction of a masseter/jaw position reflex, and correcting the elevation angle of the occlusal reference plane of a mandibular teeth dentition with respect to a horizontal reference plane so that the forced expiratory residual volume is not more than a predetermined value, for example, 500 milliliters or less. Therefore, a chewing occlusal jaw position in which a difference between the heights of the mandibular angle and the mental region is corrected can be configured by correcting the elevation angle of the occlusal reference plane of the jaw teeth dentition with respect to the horizontal reference plane according to the guidance.

(Effects of Mini-Splint)

The height difference correction guidance means guides a medical care operation of preparing a mini-splint in which the predetermined, corrected elevation angle of the occlusal reference plane of the mandibular teeth dentition with respect to the horizontal reference plane is set, and mounting the mini-splint on the mandibular teeth dentition to measure the forced expiratory residual volume with the spirometer. Therefore, when the forced expiratory residual volume of the patient does not decrease to 500 milliliters or less, a correction for increasing the elevation angle of the occlusal reference plane by mounting the mini-splint prepared in advance enables a difference between the heights of the mandibular angle and the mental region to be corrected so that the forced expiratory residual volume of the patient is 500 milliliters or less.

(Effects of Selection of Mini-Splint)

The height difference correction guidance means guides a medical care operation of preparing a plurality of kinds of mini-splints of which the elevation angle-corrected angles are different, selecting a mini-splint having a predetermined elevation angle-corrected angle from the mini-splints, and using the mini-splint. Therefore, the selective mounting of the plurality of kinds of the mini-splints of which the elevation angle-corrected angles are different on the mandibular teeth dentition of the patient and the measurement of a forced expiratory residual volume with a spirometer enable a correction for increasing the elevation angle of the occlusal reference plane to be easily performed and a difference between the heights of the mandibular angle and the mental region to be corrected so that the forced expiratory residual volume is 500 milliliters or less.

(Effects of Adjustment of Anteroposterior Positions of Forward Bending Position and Backward Bending Position)

The anteroposterior position adjustment guidance means guides a medical care operation of finely adjusting the anteroposterior position of a chewing occlusal jaw position so that the head position of the patient in rest and relaxation is maintained at a midline position with respect to the visual line of the patient. Therefore, the medical care operation according to the guidance enables the head position of the patient bent forward or backward in the rest and relaxation to be finely adjusted at a proper position maintained at the midline position with respect to the visual line of the patient and the basal muscle tones of the anterior and posterior surfaces of the body to be balanced.

(Effects Due to Assistance of Adjustment of Anteroposterior Position)

The anteroposterior position adjustment guidance means guides a medical care operation of moving forward a mandibular position when the head position of the patient in the rest and relaxation is a forward bending position and moving backward the mandibular position when the head position is a backward bending position. More specifically, in a case in which the patient is incapable of single-handedly finely adjusting an anteroposterior position, the anteroposterior position adjustment guidance means guides a medical care operation of putting wax on the lingual tooth surface of a mandibular anterior teeth portion to adjust the thickness of the wax so that forward movement is assisted when the head position of the patient in the rest and relaxation is a forward bending position, and guides a medical care operation of putting wax on the lingual tooth surface of a maxillary anterior teeth portion to adjust the thickness of the wax so that backward movement is assisted when the head position of the patient in the rest and relaxation is a backward bending position. Therefore, the application of the wax and the adjustment of the thickness according to the guidance enable the head position of the patient bent forward or backward in the rest and relaxation to be finely adjusted at a proper position maintained at the midline position with respect to the visual line of the patient and the basal muscle tones of the anterior and posterior surfaces of the body to be balanced.

(Effects of Mouthpiece Production Guidance)

The mouthpiece production guidance means guides production of a mouthpiece part on which the maxillary teeth dentition is carved based on the maxillary model, and which has an approximately uniform thickness, then guides an operation of sandwiching the optimum occlusal impression member between the jaw model and the mandibular model to fix the jaw model and the mandibular model to an occludator to reproduce the optimum chewing occlusal jaw position, and then guides an operation of mounting the mouthpiece part on the maxillary model of the occludator from which the optimum occlusal impression member is removed, and applying the teeth dentition impression of the mandibular model to an occlusal surface of the mouthpiece part based on occlusion with the mandibular model via the mouthpiece part to complete a mouthpiece. Therefore, the mouthpiece including an occlusal surface on which the teeth dentition impression of the mandibular model is formed can be easily produced using the occludator on the basis of the optimum occlusal impression member on which the impressions of a maxillary teeth dentition and a mandibular teeth dentition in the optimum chewing occlusal jaw position that balances the systemic basal muscle tone of the patient are carved.

(Effects of Guidance of Fine Adjustment of Mouthpiece Corresponding to Correction of Position of Mandibular Angle)

The mouthpiece fine adjustment guidance means, in the state of the resting jaw position of the patient in which the completed mouthpiece is mounted on the maxillary teeth dentition of the patient, guides a fine adjustment operation of polishing the thickness of the mouthpiece located on the lingual tooth surface of a maxillary right molar tooth to reduce the thickness of the mouthpiece so that the laterality of the basal muscle tone of the posterior cervical region is eliminated when the enhancement of the basal muscle tone of the right of the posterior cervical region is input by a screen operation through the display, guides a fine adjustment operation of polishing the thickness of the mouthpiece located on the lingual tooth surface of the maxillary left molar tooth to reduce the thickness of the mouthpiece so that the laterality of the basal muscle tone of the posterior cervical region is eliminated when the enhancement of the basal muscle tone of the left of the posterior cervical region is input by a screen operation through the display, and guides a fine adjustment operation of performing no fine adjustment by polishing of the mouthpiece when no enhancement of the basal muscle tone of the posterior cervical region is input by a screen operation through the display and there is no laterality. Therefore, the fine adjustment operations of polishing the mouthpiece according to the guidances allows induction to the proper position of the mandibular angle in which the basal muscle tone of the posterior cervical region of the patient in the resting jaw position state to be balanced due to a tongue/jaw position reflex, and enables the enhancement of the basal muscle tone of the posterior cervical region to be eliminated.

(Effects of Guidance of Fine Adjustment of Mouthpiece Corresponding to Correction of Position of Mental Region)

The mouthpiece fine adjustment guidance means, in the state of the resting jaw position of the patient in which the completed mouthpiece is mounted on the maxillary teeth dentition of the patient, guides a fine adjustment operation of polishing the thickness of the mouthpiece located on the lingual tooth surface of a maxillary right premolar tooth to reduce the thickness of the mouthpiece so that the laterality of the basal muscle tone of the anterior cervical region is eliminated when the enhancement of the basal muscle tone of the right of the anterior cervical region is input by a screen operation through the display, guides a fine adjustment operation of polishing the thickness of the mouthpiece located on the lingual tooth surface of the maxillary left premolar tooth to reduce the thickness of the mouthpiece so that the laterality of the basal muscle tone of the anterior cervical region is eliminated when the enhancement of the basal muscle tone of the left of the anterior cervical region is input by a screen operation through the display, and guides a fine adjustment operation of performing no fine adjustment by polishing of the mouthpiece when no enhancement of the basal muscle tone of the anterior cervical region is input by a screen operation through the display and there is no laterality. Therefore, the fine adjustment operations of polishing the mouthpiece according to the guidances allows induction to the proper position of the mandibular angle in which the basal muscle tone of the anterior cervical region of the patient in the resting jaw position state to be balanced due to a tongue/jaw position reflex, and enables the enhancement of the basal muscle tone of the anterior cervical region to be eliminated.

(Effects of Guidance of Fine Adjustment of Mouthpiece Corresponding to Adjustment of Anteroposterior Position)

The mouthpiece fine adjustment guidance means, in the resting jaw position state of the patient in which the completed mouthpiece is mounted on the maxillary teeth dentition of the patient, guides a fine adjustment operation of polishing the thickness of the mouthpiece located on the lingual tooth surface of a maxillary anterior teeth portion to reduce the thickness of the mouthpiece so that the forward movement of the mandible is assisted when an input in that the head position of the patient in rest and relaxation is a forward bending position is performed by a screen operation through the display, and guides a fine adjustment operation of increasing the thickness of the mouthpiece located on the lingual tooth surface of the maxillary anterior teeth portion to increase the thickness of the mouthpiece so that the backward movement of the mandible is assisted when an input in that the head position of the patient in the rest and relaxation is a backward bending position is performed by a screen operation through the display. Therefore, the polishing or thickening of the mouthpiece according to the guidance enables the head position of the patient bent forward or backward in the rest and relaxation to be finely adjusted at a proper forward or backward position maintained at the midline position with respect to the visual line of the patient and the basal muscle tones of the anterior and posterior surfaces of the body to be balanced.

(Effects Due to Adjustment of Thickness in Micron Order)

The mouthpiece fine adjustment guidance means guides a fine adjustment operation of changing the thickness of a mouthpiece body in a unit of micron order. Therefore, the mouthpiece is constructed so that proper chewing occlusal and resting occlusal states in which the systemic basal muscle tone is basically balanced are obtained in a state before the adjustment, and, in addition, for example, a decrease in the adjustment for the balance in a unit of micron order by the polishing of the thickness of the mouthpiece body allows an error between the production and mounting of the mouthpiece to be eliminated, and enables the basal muscle tone to be more reliably balanced.

(Effects of Tool Guidance)

Tool guidance means that guides a predetermined tool used for a treatment operation for producing the mouthpiece is included, and, for example, the tool guidance means guides: a posture measurement instrument that measures the posture of a patient; and an electromyograph that measures the enhancement of the basal muscle tones of the posterior and anterior cervical regions of the patient. Therefore, the measurement and evaluation of the posture before and after treatment by the posture measurement instrument enables the treatment effect of recovering the balance of the basal muscle tone of the patient to maintain a proper posture by mounting the mouthpiece to be confirmed. The use of the electromyograph enables the enhancement of the basal muscle tones of the posterior cervical region and the anterior cervical region to be confirmed, allows the results of diagnosis with palpation to be more reliable, and enables the precision of the treatment operation of the correction of the positions of the mandibular angle and the mental region to be enhanced.

(Third Invention: Effects of Mini-Splint)

In another embodiment of the present invention, a mini-splint is used in construction of an optimized chewing occlusal jaw position that balances the systemic basal muscle tone of a patient, and the mini-splint includes: a fixation portion that is detachably fixedly mounted between at least a second premolar tooth and a first molar tooth in the mandibular teeth dentition of the patient; and an angle-corrected surface that is disposed integrally with the fixation portion and fitted to the occlusal surface of the first molar tooth to set a predetermined elevation angle-corrected angle with respect to the predetermined buccal cusp of the first molar tooth. Therefore, when the optimized chewing occlusal jaw position of the patient, balancing the systemic basal muscle tone, is temporarily constructed, the mounting of the mini-splint allows a proper occlusal reference plane in which the elevation angle is corrected to be easily formed, thereby enabling a chewing occlusal jaw position with an optimized difference between the heights of the mental region and the mandibular angle to be configured.

In this case, wax is put on the occlusal surfaces of the both maxillary first molar teeth in a state in which the mandibular position and the mental region are corrected at the proper positions to cancel the lateral imbalance of the basal muscle tones of the posterior cervical region and the anterior cervical region, the chewing position jaw position is constructed with respect to the occlusal reference plane with the corrected elevation angle of the mini-splint mounted on the mandibular teeth dentition, and a forced expiratory residual volume is measured with a spirometer after the induction of a masseter/jaw position reflex. In a case in which the forced expiratory residual volume desirably decreases to 500 milliliters or less, the imbalance of the basal muscle tone in a forward bending posture or a backward bending posture can be canceled and changed to a favorable balance.

Herein, the forced expiratory residual volume refers to the volume of air remaining in the lung when both the expiratory muscle and the inspiratory muscle are relaxed after maximum inspiration, and is measured with the spirometer. Typically, exhalation of most air from the interior of the lung only by relaxation is considered to be ideal breathing balance.

(Effects of Mini-Splints Having Different Elevation Angles)

A plurality of kinds of mini-splints of which the elevation angle-corrected angles due to the angle-corrected surface are different are prepared, and a mini-splint having a predetermined elevation angle-corrected angle is selected from the mini-splints and fixedly mounted between at least a second molar tooth and a first premolar tooth in the mandibular teeth dentition of the patient through the fixation portion. For example, a plurality of kinds of mini-splints having elevation angle-corrected angles of 7°, 14°, 21°, and 28° are prepared, and a mini-splint is selected and mounted so that an elevation angle-corrected angle is increased in turn to 7°, 14°, 21°, and 28° to allow a forced expiratory residual volume to decrease to 500 milliliters or less, whereby the elevation angle-corrected angle can be easily changed to decrease the forced expiratory residual volume.

(Fourth Invention: Effects of Method for Producing Mouthpiece)

The present invention is to provide a method for producing a mouthpiece that is detachably mounted on the maxillary teeth dentition of a patient to configure an optimized chewing occlusal jaw position that balances the systemic basal muscle tone of the patient, the method including: a maxillary and mandibular models production procedure of producing a maxillary model and a mandibular model on which the teeth dentitions of the patient are arranged; a mandibular angle position correction procedure of correcting the lateral position of a mandibular angle at an optimized position using the tongue/jaw position reflex of the patient; a mental region position correction procedure of correcting the lateral position of the mental region at an optimized position using the tongue/jaw position reflex of the patient; a height difference correction procedure of correcting a difference between the heights of the mandibular angle and the mental region using the masseter/jaw position reflex of the patient in a state in which corrections according to the mandibular angle position correction procedure and the mental region position correction procedure have been finished; an anteroposterior position adjustment procedure of adjusting the anteroposterior position of a mandibular position to an optimized position in a state in which a correction according to the height difference correction procedure has been finished; an optimum occlusion taking procedure of producing an optimum occlusal impression member on which the occlusal teeth dentitions of the optimized chewing occlusal jaw position are impressed in a state in which a chewing occlusal jaw position optimized for the patient is constructed by the anteroposterior position adjustment procedure; an occlusal model production procedure of constructing an optimized chewing occlusal model by fixing the optimum occlusal impression member produced in the optimum occlusion taking procedure to an occludator in a state in which the optimum occlusal impression member is sandwiched between the teeth dentitions of the maxillary model and the mandibular model; a mouthpiece production procedure of producing a mouthpiece on which the teeth dentition impression of a maxillary tooth shape and the teeth dentition impression of a mandibular tooth shape are formed, so that an optimized chewing occlusal jaw position is achieved, by the occludator in which the chewing occlusal model is constructed, and which has an approximately uniform thickness; and a mouthpiece fine adjustment procedure of performing fine adjustment so that the systemic basal muscle tone of the patient is balanced to obtain an optimum resting jaw position in the resting jaw position state of the patient on which the mouthpiece produced in the mouthpiece production procedure is mounted. Therefore, effects similar to the effects of the system for supporting the production of the mouthpiece according to the second invention described above can be obtained, and thus, a description thereof is omitted. The same also applies to a specific aspect of amethod for producing a mouthpiece.

### BRIEF DESCRIPTION OF THE DRAWINGS

FIG. 1 is a flow diagram illustrating a procedure for producing a mouthpiece according to the present invention.

FIG. 2 is an explanatory diagram illustrating a brain-stem reflex to the change of the form of a jaw position based on production of a mouthpiece.

FIG. 3 is an explanatory diagram illustrating the maxillary and mandibular models of a patient.

FIG. 4 is a flow diagram illustrating the details of the correction of the mandibular angle in step S2 of FIG. 1.

FIG. 5 is an explanatory diagram illustrating a first pattern without laterality between the basal muscle tones of the posterior cervical region and the anterior cervical region and without the correction of the position of the mandibular angle.

FIG. 6 is an explanatory diagram illustrating a second pattern in which the position of the mandibular angle is corrected in the occurrence of the enhancement of the basal muscle tone in the right of the posterior cervical region.

FIG. 7 is an explanatory diagram illustrating a third pattern in which the position of the mandibular angle is corrected in the occurrence of the enhancement of the basal muscle tone in the left of the posterior cervical region.

FIG. 8 is a flow diagram illustrating the details of the correction of the position of the mental region in step S3 of FIG. 1.

FIG. 9 is an explanatory diagram illustrating a fourth pattern in which the position of the mental region is corrected in the occurrence of the enhancement of the basal muscle tone in the right of the anterior cervical region.

FIG. 10 is an explanatory diagram illustrating a fifth pattern in which the position of the mental region is corrected in the occurrence of the enhancement of the basal muscle tone in the left of the anterior cervical region.

FIG. 11 is an explanatory diagram illustrating a sixth pattern in which the positions of the mandibular angle and the mental region are corrected in the occurrence of the enhancement of the basal muscle tone in the right of the posterior cervical region and the left of the anterior cervical region.

FIG. 12 is an explanatory diagram illustrating a seventh pattern in which the positions of the mandibular angle and the mental region are corrected in the occurrence of the enhancement of the basal muscle tone in the right of the posterior cervical region and the right of the anterior cervical region.

FIG. 13 is an explanatory diagram illustrating an eighth pattern in which the positions of the mandibular angle and the mental region are corrected in the occurrence of the enhancement of the basal muscle tone in the left of the posterior cervical region and the right of the anterior cervical region.

FIG. 14 is an explanatory diagram illustrating a ninth pattern in which the positions of the mandibular angle and the mental region are corrected in the occurrence of the enhancement of the basal muscle tone in the left of the posterior cervical region and the left of the anterior cervical region.

FIG. 15 is a flow diagram illustrating the details of a procedure of correcting a difference between the heights of the mandibular angle and the mental region in step S4 of FIG. 1.

FIG. 16 is an explanatory diagram illustrating a mini-splint used for correcting a difference between the heights of the mandibular angle and the mental region.

FIG. 17 is an explanatory diagram of mini-splints of which the different correction elevation angles are set.

FIG. 18 is a flow diagram illustrating the details of a procedure of the anteroposterior fine adjustment of the mandibular position in step S5 of FIG. 1.

FIG. 19 is an explanatory diagram illustrating a forward movement pattern in which the mandible is moved forward when the head position in rest and relaxation is bent forward.

FIG. 20 is an explanatory diagram illustrating a backward movement pattern in which the mandible is moved backward when the head position in rest and relaxation is bent backward.

FIG. 21 is an explanatory diagram illustrating an occludator in which maxillary and mandibular models are fixed using an optimum occlusal teeth mark member taken from an optimized chewing occlusal state.

FIG. 22 is an explanatory diagram illustrating production of a mouthpiece according to the present invention using the occludator illustrated in FIG. 21.

FIG. 23 is an explanatory diagram illustrating the plane, side surface, and bottom surface of the mouthpiece according to the present invention produced using the occludator of FIG. 22.

FIG. 24 is a flow diagram illustrating a procedure of finely adjusting the mouthpiece illustrated in FIG. 23.

FIG. 25 is a flow diagram illustrating the details of the fine adjustment of the mouthpiece corresponding to the mandibular angle in step S52 of FIG. 24.

FIG. 26 is an explanatory diagram illustrating a first fine adjustment pattern without the laterality of the basal muscle tones of the posterior cervical region and the anterior cervical region and without the fine adjustment of a mouthpiece.

FIG. 27 is an explanatory diagram illustrating a second fine adjustment pattern in which a mouthpiece is finely adjusted in the occurrence of the enhancement of the basal muscle tone in the right of the posterior cervical region.

FIG. 28 is an explanatory diagram illustrating a third fine adjustment pattern in which a mouthpiece is finely adjusted in the occurrence of the enhancement of the basal muscle tone in the left of the posterior cervical region.

FIG. 29 is a flow diagram illustrating the details of the fine adjustment of a mouthpiece corresponding to the mental region in step S53 of FIG. 24.

FIG. 30 is an explanatory diagram illustrating a fourth fine adjustment pattern in which a mouthpiece is finely adjusted in the occurrence of the enhancement of the basal muscle tone in the right of the anterior cervical region.

FIG. 31 is an explanatory diagram illustrating a fifth fine adjustment pattern in which a mouthpiece is finely adjusted in the occurrence of the enhancement of the basal muscle tone in the left of the anterior cervical region.

FIG. 32 is an explanatory diagram illustrating a sixth fine adjustment pattern in which a mouthpiece is finely adjusted in the occurrence of the enhancement of the basal muscle tone in the right of the posterior cervical region and the left of the anterior cervical region.

FIG. 33 is an explanatory diagram illustrating a seventh fine adjustment pattern in which a mouthpiece is finely adjusted in the occurrence of the enhancement of the basal muscle tone in the right of the posterior cervical region and the right of the anterior cervical region.

FIG. 34 is an explanatory diagram illustrating an eighth fine adjustment pattern in which a mouthpiece is finely adjusted in the occurrence of the enhancement of the basal muscle tone in the left of the posterior cervical region and the right of the anterior cervical region.

FIG. 35 is an explanatory diagram illustrating a ninth fine adjustment pattern in which a mouthpiece is finely adjusted in the occurrence of the enhancement of the basal muscle tone in the left of the posterior cervical region and the left of the anterior cervical region.

FIG. 36 is a flow diagram illustrating the details of the fine adjustment of the mouthpiece corresponding to the anteroposterior position of the mandibular position in step S54 of FIG. 24.

FIG. 37 is an explanatory diagram illustrating a forward movement fine adjustment pattern in which a mouthpiece is finely adjusted when the head position in rest and relaxation is bent forward.

FIG. 38 is an explanatory diagram illustrating a backward movement fine adjustment pattern in which a mouthpiece is finely adjusted when a head position in rest and relaxation is bent backward.

FIG. 39 is a block diagram illustrating an embodiment of a system for supporting production of a mouthpiece.

FIG. 40 is a block diagram illustrating the details of a chewing occlusal jaw position guidance portion.

FIG. 41 is a block diagram illustrating the details of a mouthpiece fine adjustment guidance portion.

FIG. 42 is an explanatory diagram illustrating a guidance screen of a main menu.

FIG. 43 is an explanatory diagram illustrating a guidance screen for correcting the mandibular angle and the mental region, displayed when a chewing occlusal jaw position is selected.

FIG. 44 is an explanatory diagram illustrating a guidance screen for correcting a difference between the heights of the mandibular angle and the mental region.

FIG. 45 is an explanatory diagram illustrating a guidance screen for assisting the forward movement of the chewing jaw position.

FIG. 46 is an explanatory diagram illustrating a guidance screen for assisting the backward movement of the chewing jaw position.

FIG. 47 is an explanatory diagram illustrating a guidance screen for taking optimum occlusion.

FIG. 48 is an explanatory diagram illustrating a guidance screen for producing an occlusal model.

FIG. 49 is an explanatory diagram illustrating a guidance screen for producing a mouthpiece.

FIG. 50 is an explanatory diagram illustrating a guidance screen for performing fine adjustment of a mouthpiece, corresponding to the position of the mandibular angle and the position of the mental region.

FIG. 51 is an explanatory diagram illustrating a guidance screen for performing fine adjustment of a mouthpiece, corresponding to the assistance of the forward movement of the chewing jaw position.

FIG. 52 is an explanatory diagram illustrating a guidance screen for performing fine adjustment of a mouthpiece, corresponding to the assistance of the backward movement of the chewing jaw position.

FIG. 53 is an explanatory diagram illustrating a guidance screen for a tool, displayed by a tool guidance portion.

FIG. 54 is an explanatory diagram illustrating a guidance screen for a posture measurement meter.

FIG. 55 is an explanatory diagram illustrating a guidance screen for an electromyograph.

### BEST MODE FOR CARRYING OUT THE INVENTION

[Scheme of Production of Mouthpiece]

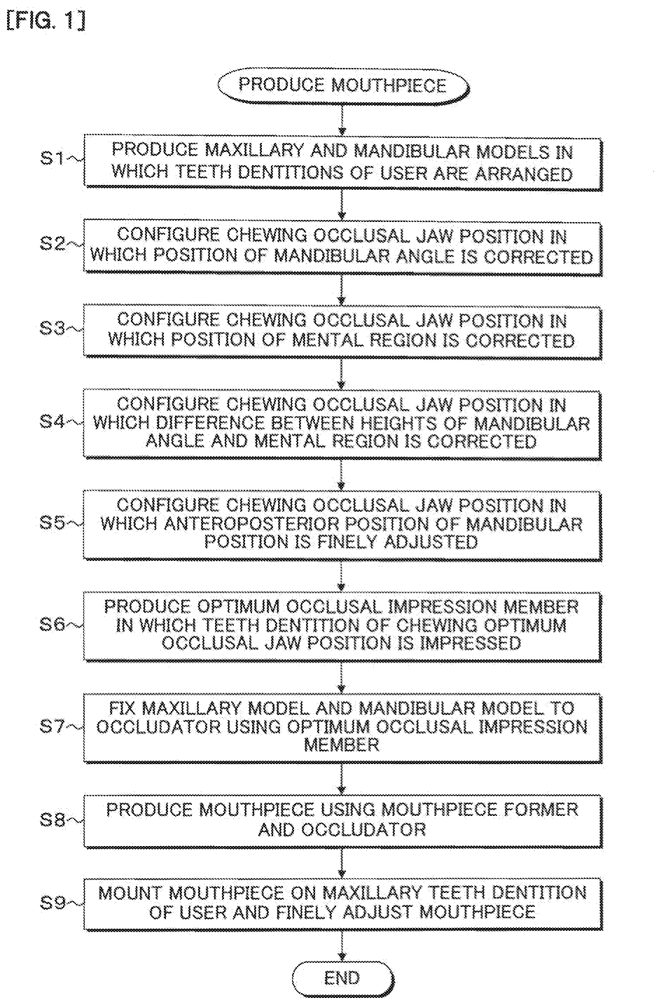

FIG. 1 is a flow diagram illustrating the scheme of a procedure for producing a mouthpiece according to the present invention. The procedure in the flow diagram of FIG. 1, divided into steps S1 to S9, is illustrated. The steps are chronologically proceeding operation procedures, which may be expressed as processes, stages, phases, or the like instead of steps. However, steps are used in the present embodiment.

In the present embodiment, a mouthpiece that corrects the mandibular position (the position of the mandible) in order to optimize the balance of the systemic basal muscle tone of, for example, a patient with fibromyalgia is produced according to subsequent steps S1 to S9.

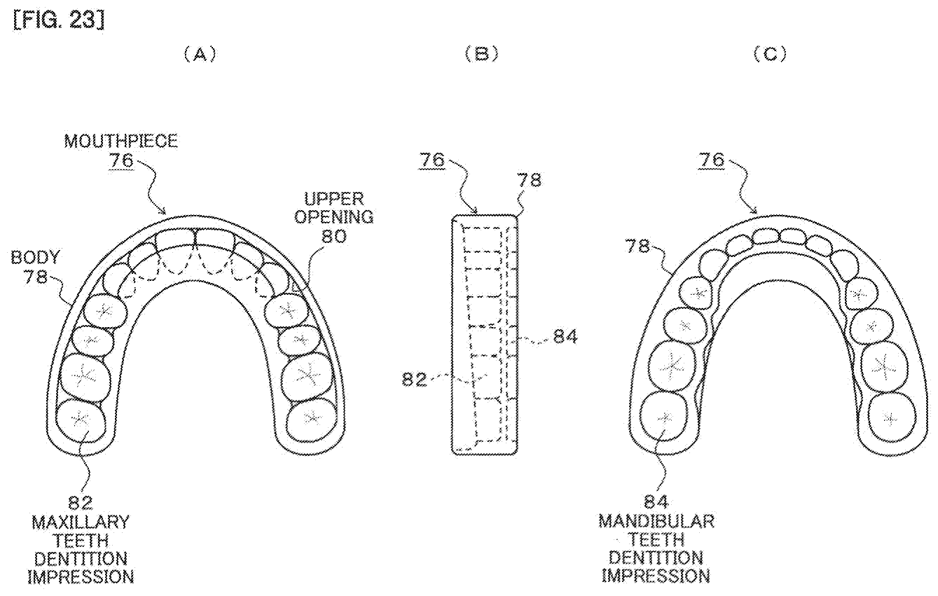

In a mouthpiece detachably mounted on the maxillary teeth dentition of a patient according to steps S1 to S9, as illustrated in FIG. 23, a maxillary teeth dentition impression 82 due to a maxillary model is carved in an upper opening 80 of a mouthpiece body 78 having an approximately uniform thickness in a mouthpiece 76, and a mandibular teeth dentition impression 84 due to a mandibular model in an optimized chewing occlusal jaw position that balances the systemic basal muscle tone of the patient is carved on a bottom surface (occlusal surface) of the mouthpiece body 78.

The proper occlusion positional relationship of the mandible with respect to the maxilla, in which optimized chewing occlusal and resting occlusal states are achieved, is reproduced by mounting the mouthpiece 76 constituted in such a manner on the maxillary teeth dentition of the patient.

In step S1 which is a maxillary and mandibular models production procedure, maxillary and mandibular models on which the teeth dentitions of the patient are arranged are produced. In step S2 which is a mandibular angle position correction procedure, a chewing occlusal jaw position at which the position of the mandibular angle of the patient is corrected is configured. In step S3 which is a mental region position correction procedure, a chewing occlusal jaw position at which the position of the mental region of the patient is corrected is configured. In step S4 which is a height difference correction, a chewing occlusal jaw position in which a difference between the heights of the mandibular angle and the mental region of the patient is corrected is configured in a state in which the corrections of steps S2 and S3 have been finished. In step S5 which is an anteroposterior position adjustment procedure, a chewing occlusal jaw position at which the anteroposterior position of the mandibular position is finely adjusted is configured in a state in which the correction of step S4 has been finished.

An optimized chewing occlusal jaw position that balances a systemic basal muscle tone is constructed by such procedures of steps S2 to S5.

Subsequently, in step S6 which is an optimum occlusion taking procedure, an optimum occlusal impression member on which the occlusal teeth dentition of the optimized chewing occlusal jaw position is impressed is produced using a bite material in a state in which the optimized chewing occlusal jaw position is configured in steps S2 to S5.

In step S7 which is an occlusal model production procedure, the optimum occlusal impression member produced in step S6 is sandwiched between the teeth dentitions of the maxillary and mandibular models produced in step S1 to configure an optimized chewing occlusal jaw position, which is fixed to an occludator.

In step S8 which is a mouthpiece production procedure, a mouthpiece part having a certain thickness with respect to the maxillary model is produced using a mouthpiece former, and subsequently, the mandibular teeth dentition is impressed on the mouthpiece part by the occludator to which the maxillary model and the mandibular model are fixed so that the optimized chewing occlusal jaw position is achieved, to produce a mouthpiece.

In step S9 which is a mouthpiece fine adjustment procedure, the mouthpiece produced in step S8 is mounted on the maxillary teeth dentition of the patient and finely adjusted by, for example, polishing of the lingual tooth surface of the mouthpiece so that an adequate resting jaw position is achieved, to optimize both the chewing occlusal jaw position and the resting jaw position, and a mouthpiece in which appropriate jaw movement is further maintained even in opening and closing mouth movement is completed.

[Configuration of Chewing Occlusal Jaw Position in Which Balance of Systemic Basal Muscle Tone is Optimized]

Prior to a description of steps S1 to S9, the findings of the present inventor that the balance of the systemic basal muscle tone is optimized by allowing the mandibular bone to be at a proper position will be described.

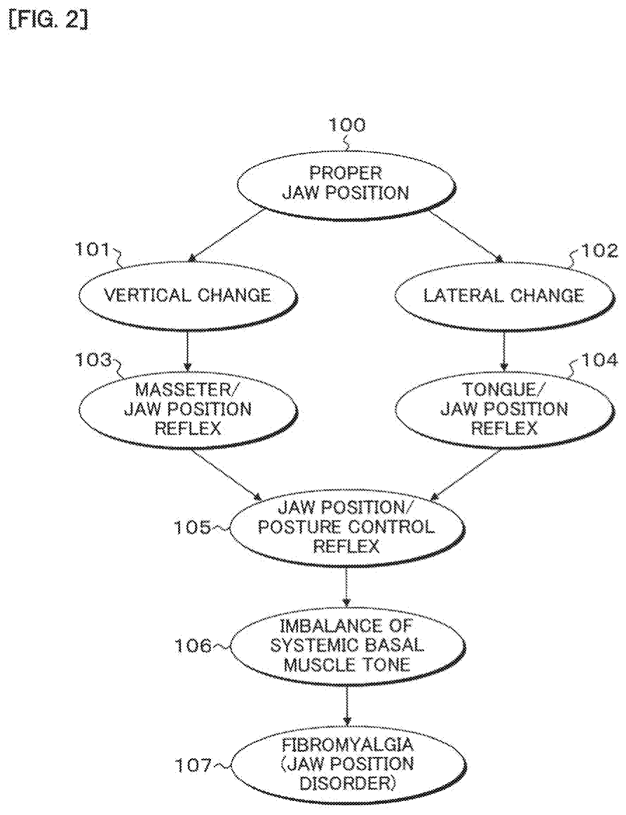

FIG. 2 is an explanatory diagram illustrating a brain-stem reflex to the change of the form of a jaw position based on production of the mouthpiece according to the present embodiment.

The present inventor considers that the occurrence of the abnormality of the position of the mandibular bone results in the imbalance of a systemic basal muscle tone. As illustrated in FIG. 2, wear in a teeth due to aging, an injury caused by a traffic accident or the like, dental practice, or the like causes the position of the mandibular bone to deviate from a normal position, thereby changing the relative positional relationship of the mandibular bone with respect to the cranial bone, although the systemic basal muscle tone is balanced in a state in which a proper jaw position 100 in which the mandibular bone is at a proper position with respect to the cranial bone is obtained.

Such a change of the position of the mandibular bone is classified roughly into a vertical change 101 and a lateral change 102. As a matter of course, the vertical change 101 and the lateral change 102 occurs not only simultaneously but also compositely. In response to the vertical change 101, the position of the jaw is moved to a position determined, by the central nervous system, to be more favorable for chewing, by a masseter/jaw position reflex 103. In other words, the vertical change of the mandibular bone is transmitted from the positon and motion senses of the muscle associated with chewing to the central nervous system through the sensory fibers of the trigeminal nerve, to move the position of the jaw to a position determined, by the central nervous system, to be more favorable for chewing.

The lateral change 102 is sensed by the touch sense of the trigeminal nerve through the tongue due to a tongue/jaw position reflex 104, transmitted to the central nervous system, to move the position of the jaw to a position determined, by the central nervous system, to be more favorable for chewing.

Since both the masseter/jaw position reflex 103 and the tongue/jaw position reflex 104 are brain-stem reflexes, a jaw position reflex occurs simultaneously with a change in the form of a teeth, and the change of the position of the jaw results in a jaw position/posture control reflex 105, causing the balance of the systemic muscle to be changed, thereby resulting in imbalance 106 of a basal muscle tone.

The name of a disease diagnosed when a balance disorder leading to the imbalance 106 of the systemic basal muscle tone caused in such a manner persistently continues for a long period, and systemic pain is intensively complained can be considered to be fibromyalgia 107. As a result, the true pathology of the fibromyalgia 107 can be considered to be jaw position disorder.

The results of research conducted by the present inventor confirmed that the mandibular position changed by the correction of the form of a teeth includes the following factors.

(1) Lateral Position of Mandibular Angle

There is a correlation between the posterior cervical region or the back surface of the body and the lateral balance of a basal muscle tone. The correlation is confirmed based on palpation and a symptom.

(2) Lateral Position of Mental Region

There is a correlation between the anterior cervical region or the anterior surface of the body and the lateral balance of a basal muscle tone. The correlation is diagnosed based on palpation and a symptom.

With regard to the conditions (1) and (2), a “tongue/jaw position reflex” test through sensing by the trigeminal nerve on the surface of the tongue confirms that the position of the jaw can be corrected instantly (within around several seconds).

(3) High Order Difference between Mandibular Angle and Mental Region, and Horizontal Property of Occlusal Plane

There is a correlation between a forward bending posture and the balance of a backward bending posture. A forced expiratory residual volume, regarded as an index, is adjusted to around 0 to 500 milliliters. There is a high causal relation with lumbago as a symptom.

Herein, the forced expiratory residual volume refers to the volume of air remaining in the lung when both the expiratory muscle and the inspiratory muscle are relaxed after maximum inspiration, and is measured using a spirometer. Typically, exhalation of most air from the interior of the lung only by relaxation is considered to be ideal breathing balance.

(4) Anteroposterior Position

There is a correlation between the balances of the basal muscle tones of the anterior and posterior cervical regions and the anterior and posterior surfaces of the body. Whether a head position (the position of the head) in rest and relaxation is a forward bending position or a backward bending position is regarded as an index, and a position which is a midline position is regarded as an optimal anteroposterior position. A close correlation between the horizontal property of an occlusal plane and a high order difference between the mandibular angle and the mental region is observed by, for example, the adjustment of the elevation angle of the occlusal plane (occlusal reference plane) through the trigeminal nerve and the deep sensation of the masseter muscle. Steps S1 to S9 will be described in detail below.

[Step S1: Production of Maxillary and Mandibular Models]

In step S1, the maxillary teeth dentition and mandibular teeth dentition of the patient are taken using the bite material, and the maxillary model on which the maxillary teeth dentition is arranged, and the mandibular model on which the mandibular teeth dentition is arranged are produced.

In other words, a bite material functioning as an impression material is put in the oral cavity of the patient to make the molds of the maxilla and mandible, and tooth mold models (dies) are produced. Subsequently, gypsum or the like is poured into the produced tooth mold models of the maxilla and the mandible and extracted after curing, thereby producing the maxillary model on which the maxillary teeth dentition is arranged and the mandibular model on which the mandibular teeth dentition is arranged.

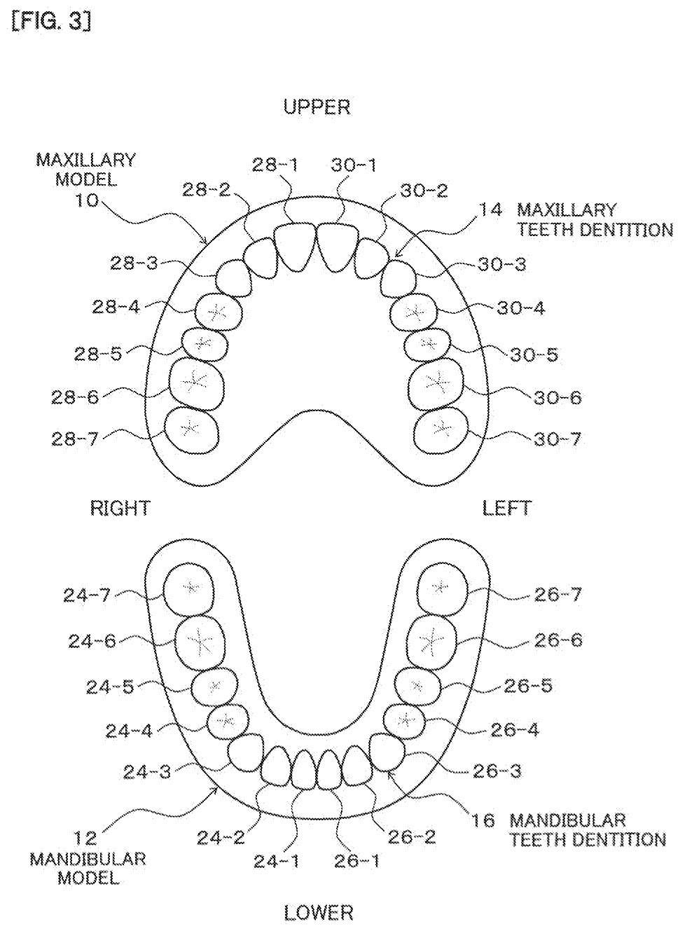

FIG. 3 is an explanatory diagram illustrating the maxillary and mandibular models of the patient. As illustrated in FIG. 3, a maxillary teeth dentition 14 taken from the patient using the bite material is arranged on a maxillary model 10. In addition, a mandibular teeth dentition 16 taken from the patient using a bite material is arranged on a mandibular model 12.

As is well known, the upper teeth dentition 14 and the lower teeth dentition 16 include the central incisors 24-1, 26-1, 28-1, and 30-1, the lateral incisors 24-2, 26-2, 28-2, and 30-2, the cuspids 24-3, 26-3, 28-3, and 30-3, the first premolar teeth 24-4, 26-4, 28-4, and 30-4, the second premolar teeth 24-5, 26-5, 28-5, and 30-5, the first molar teeth 24-6, 26-6, 28-6, and 30-6, and the second molar teeth 24-7, 26-7, 28-7, and 30-7.

[Step S2: Correction of Position of Mandible]

In Step S2 of FIG. 1, the chewing occlusal jaw position in which the position of the mandibular angle is corrected is configured. In the correction of the position of the mandibular angle, wax is primarily applied to the lingual tooth surface of the mandibular molar tooth to adjust the thickness to achieve the lateral balance while confirming the balance of the basal muscle tone of the posterior cervical region.

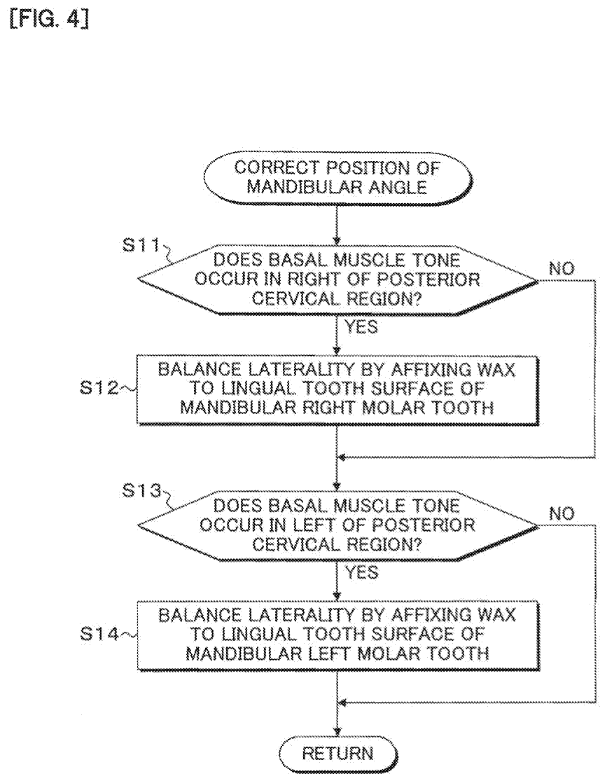

FIG. 4 is a flow diagram illustrating the details of the correction of the mandibular angle in step S2 of FIG. 1. In the correction of the position of the mandibular angle in FIG. 4, wax is applied to the lingual tooth surface of a mandibular right molar tooth to balance laterality by going to step S12 when the enhancement of a basal muscle tone occurs in the right of the posterior cervical region of the patient in step S11.

Wax is applied to the lingual tooth surface of a mandibular left molar tooth to balance laterality by going to step S14 when the enhancement of a basal muscle tone occurs in the left of the posterior cervical region of the patient in step S13.

The adjustment of balance by application of wax is not performed when there is no enhancement of a basal muscle tone in the right of the posterior cervical region of the patient in step S11, and there is no enhancement of a basal muscle tone in the left of the posterior cervical region of the patient in step S13.

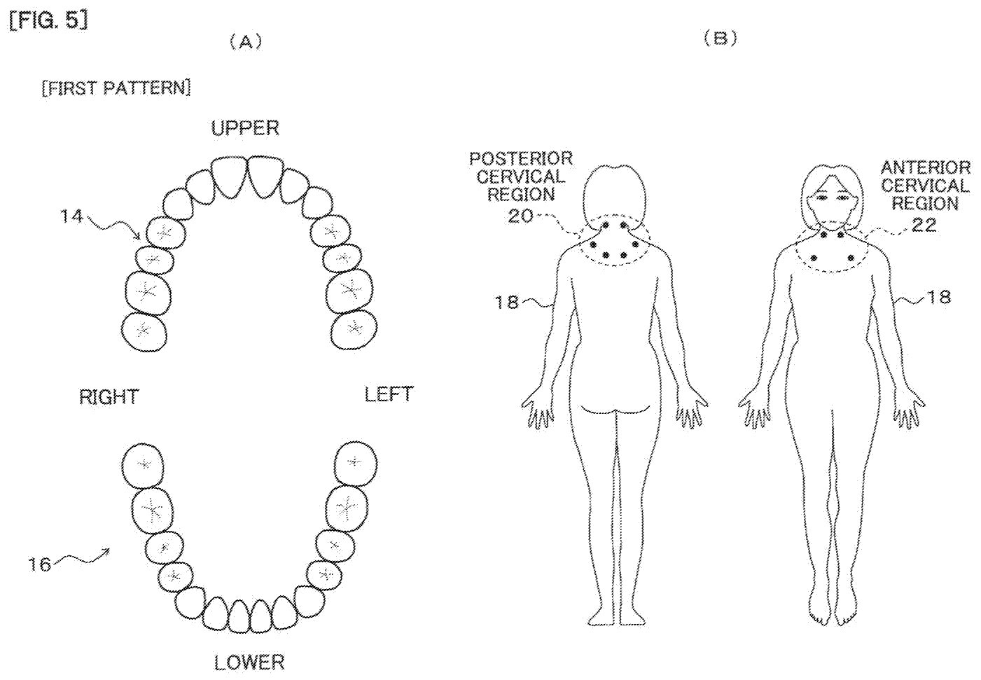

The correction of the position of the mandibular angle in FIG. 4 can be divided into the first pattern to the third pattern illustrated in FIG. 5 to FIG. 7.

(First Pattern)

FIG. 5 is an explanatory diagram illustrating a first pattern without laterality between the basal muscle tones of the posterior cervical region and the anterior cervical region and without the correction of the position of the mandibular angle. FIG. 5(A) illustrates a maxillary teeth dentition and a mandibular teeth dentition, and FIG. 5(B) illustrates the sites of occurrence of the basal muscle tones of the posterior and anterior cervical regions of the patient (the sites in which the enhancement of the basal muscle tones occurs). The black spots in FIG. 5(B) partly indicate tender points based on the classification criteria of fibromyalgia from the American College of Rheumatology.

The first pattern shows a case in which each of the posterior cervical region 20 and anterior cervical region 22 of patient 18 has no laterality and is balanced, as illustrated in FIG. 5(B). In this case, adjustment of balance by applying wax is not performed as illustrated in FIG. 5(A).

(Second Pattern)

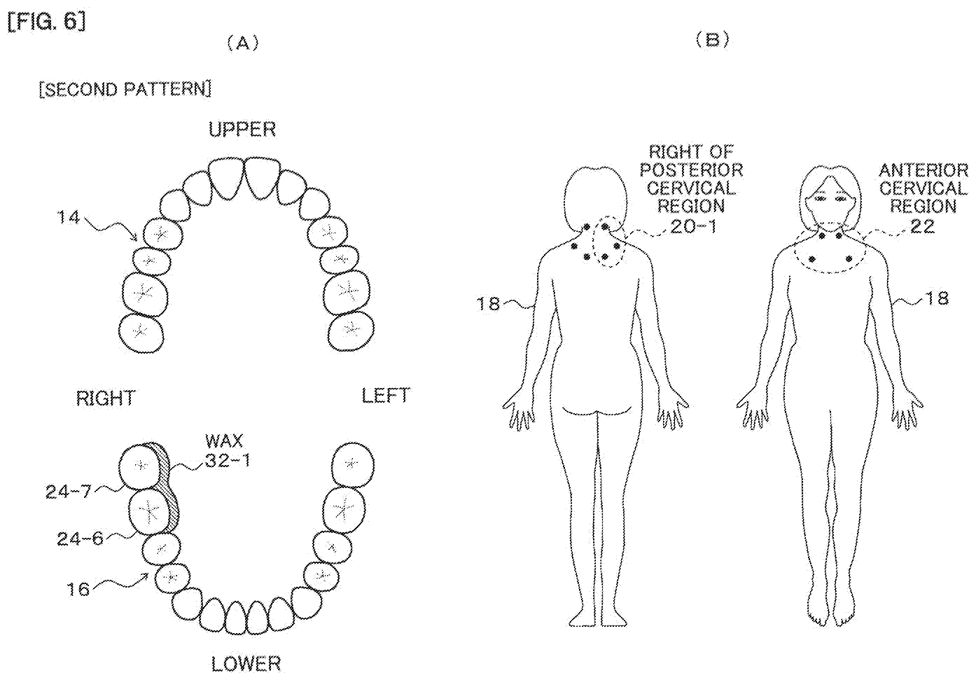

FIG. 6 is an explanatory diagram illustrating a second pattern in which the position of the mandibular angle is corrected in the occurrence of the enhancement of the basal muscle tone in the right of the posterior cervical region.

The second pattern shows the correction of a position in a case in which the enhancement of the basal muscle tone occurs in the right 20-1 of the posterior cervical region of the patient 18, and the anterior cervical region 22 of the patient 18 has no laterality and is balanced, as illustrated in FIG. 6(B). In this case, wax 32-1 is applied to the lingual tooth surfaces of the first molar tooth 24-6 and second molar tooth 24-7 of the right of the mandibular teeth dentition 16 to balance laterality, as illustrated in FIG. 6(A).

In the balance of the laterality by applying the wax 32-1, the laterality is balanced by, first, applying the thinly spread wax 32-1, inquiring of the patient 18 whether the basal muscle tone of the right 20-1 of the posterior cervical region is eliminated, and repeatedly applying the thinly spread wax 32-1 until the basal muscle tone of the right 20-1 of the posterior cervical region is eliminated. The adjustment method in which the laterality of a muscle tone at basic time is balanced by applying the wax is also similar in correction of a position by the following pattern.

(Third Pattern)

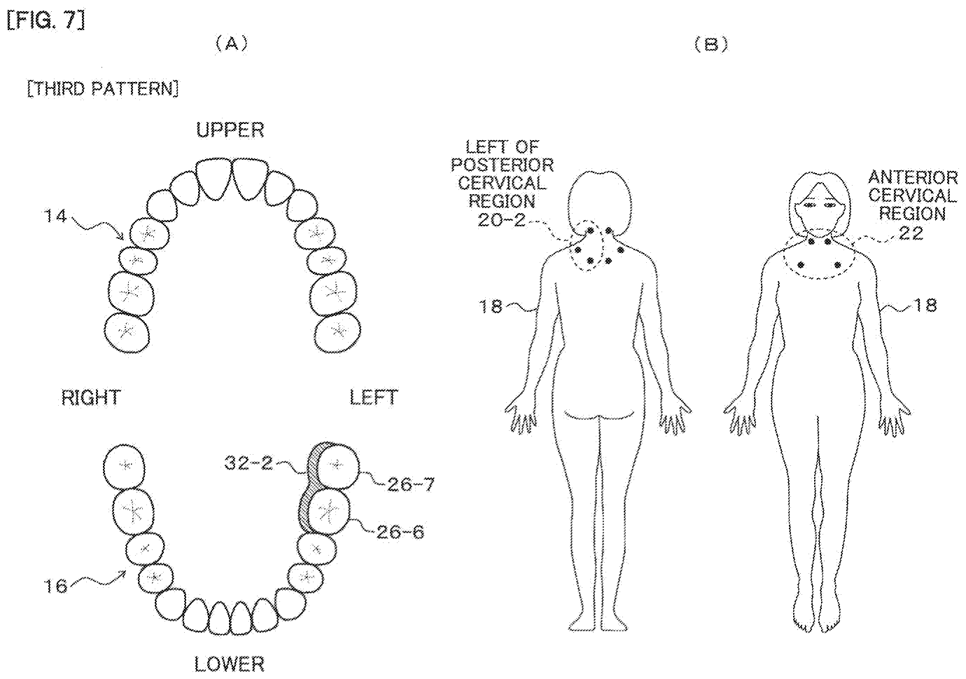

FIG. 7 is an explanatory diagram illustrating a third pattern in which the position of the mandibular angle is corrected in the occurrence of the enhancement of the basal muscle tone in the left of the posterior cervical region.

The third pattern shows the correction of a position in a case in which the enhancement of the basal muscle tone occurs in the left 20-2 of the posterior cervical region of the patient 18, and the anterior cervical region 22 of the patient 18 has no laterality and is balanced, as illustrated in FIG. 7(B). In this case, wax 32-2 is applied to the lingual tooth surfaces of the first molar tooth 26-6 and second molar tooth 26-7 of the left of the mandibular teeth dentition 16 to balance laterality, as illustrated in FIG. 7(A).

[Step S3: Correction of Position of Mental Region]

In step S3 of FIG. 1, the chewing occlusal jaw position in which the position of the mental region of the patient is corrected is configured. In the correction of the position of the mental region, wax is primarily applied to the lingual tooth surface of mandibular premolar tooth to adjust the thickness to achieve the lateral balance while confirming the balance of the basal muscle tone of the anterior cervical region.

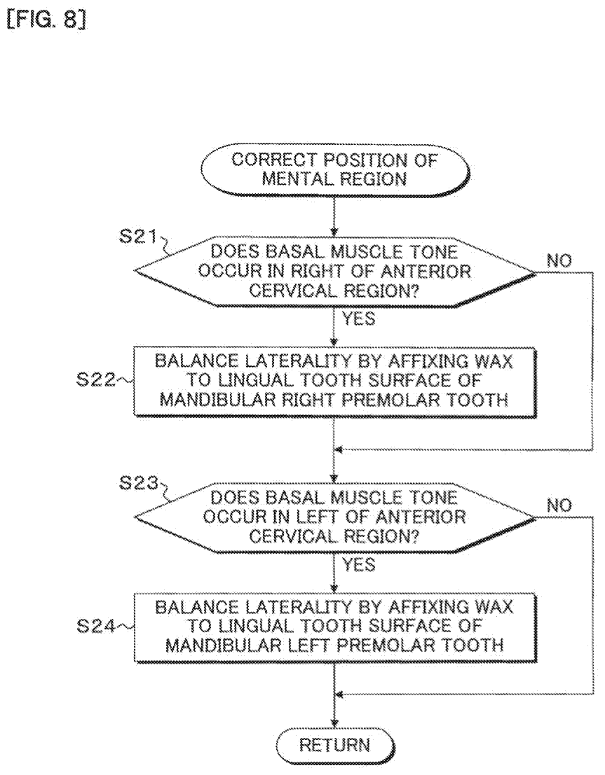

FIG. 8 is a flow diagram illustrating the details of the correction of the position of the mental region in step S3 of FIG. 1. In the correction of the position of the mental region of FIG. 8, wax is applied to the lingual tooth surface of a mandibular right premolar tooth to balance laterality by going to step S22 when the enhancement of the basal muscle tone occurs in the right of the anterior cervical region of the patient in step S21.

Wax is applied to the lingual tooth surface of a mandibular left premolar tooth to balance laterality by going to step S24 when the enhancement of the basal muscle tone occurs in the left of the anterior cervical region of the patient in step S23.

Adjustment of balance by applying wax is not performed when there is no enhancement of the basal muscle tone in the right of the anterior cervical region of the patient in step S21, and there is no enhancement of the basal muscle tone in the left of the anterior cervical region of the patient in step S23.

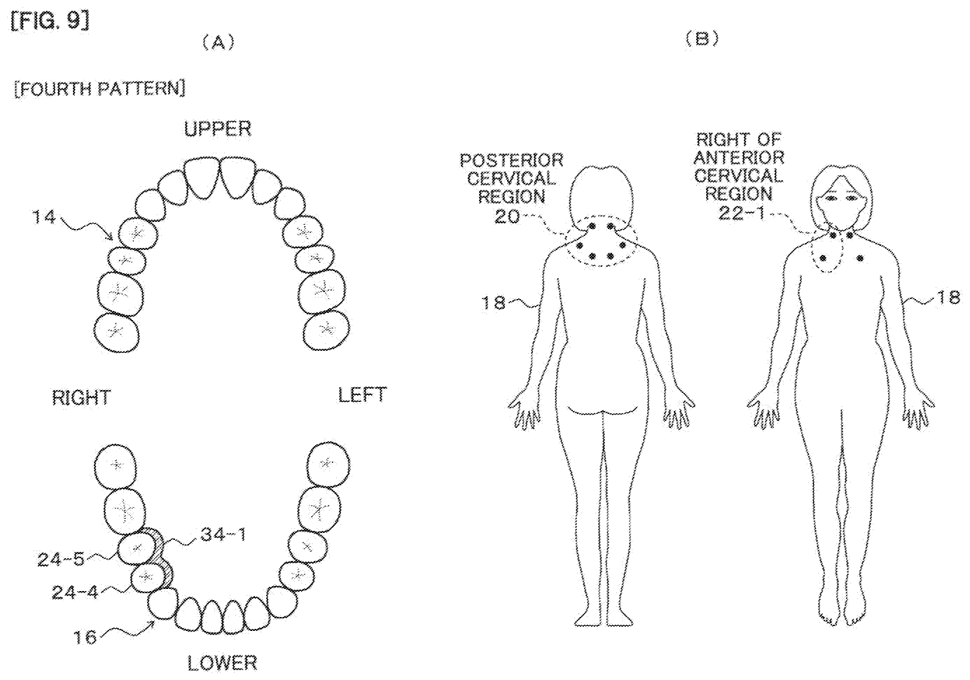

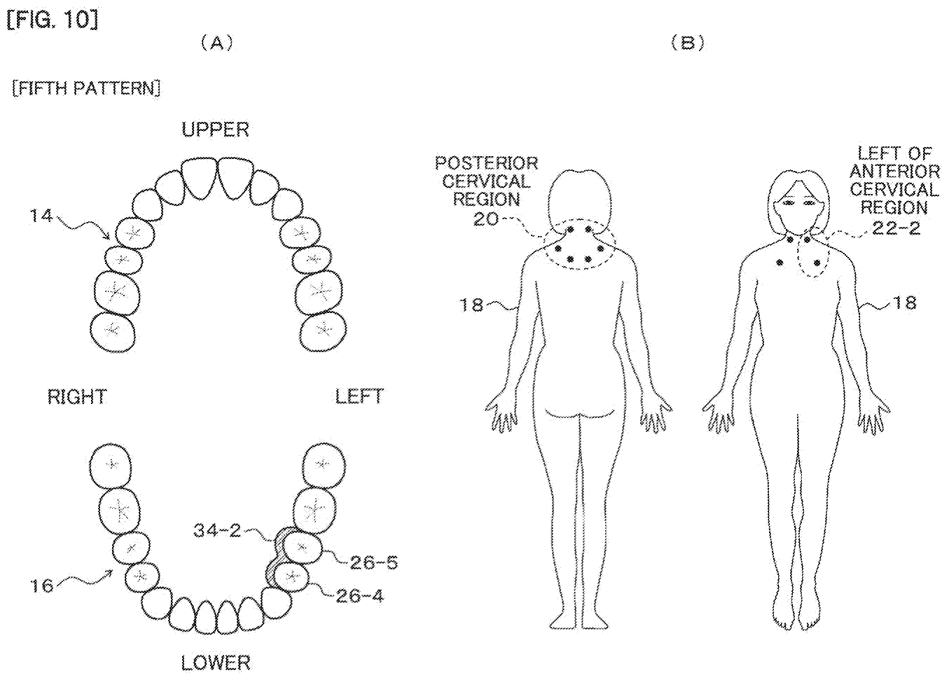

The correction of the position of the mandibular angle in FIG. 8 can be divided into a fourth pattern and a fifth pattern illustrated in FIG. 9 and FIG. 10.

(Fourth Pattern)

FIG. 9 is an explanatory diagram illustrating a fourth pattern in which the position of the mental region is corrected in the occurrence of the enhancement of the basal muscle tone in the right of the anterior cervical region.

The fourth pattern shows the correction of a position in a case in which the enhancement of the basal muscle tone occurs in the right 22-1 of the anterior cervical region of the patient 18, and the posterior cervical region 20 of the patient 18 has no laterality and is balanced, as illustrated in FIG. 9(B). In this case, wax 34-1 is applied to the lingual tooth surfaces of the first premolar tooth 24-4 and second premolar tooth 24-5 of the right of the mandibular teeth dentition 16 to balance laterality, as illustrated in FIG. 9(A).

(Fifth Pattern)

FIG. 10 is an explanatory diagram illustrating a fifth pattern in which the position of the mental region is corrected in the occurrence of the enhancement of the basal muscle tone in the left of the anterior cervical region.

The fifth pattern shows the correction of a position in a case in which the enhancement of the basal muscle tone occurs in the left 22-2 of the anterior cervical region of the patient 18, and the posterior cervical region 20 of the patient 18 has no laterality and is balanced, as illustrated in FIG. 10(B). In this case, wax 34-2 is applied to the lingual tooth surfaces of the first premolar tooth 26-4 and second premolar tooth 26-5 of the left of the mandibular teeth dentition 16 to balance laterality, as illustrated in FIG. 10(A).

[Correction of Position in Combination of Mandibular Angle and Mental Region]

The corrections of positions in combination of the correction of the position of the mandibular angle in FIG. 4 and the correction of the position of the mental region in FIG. 8 are included in sixth to ninth patterns described below.

(Sixth Pattern)

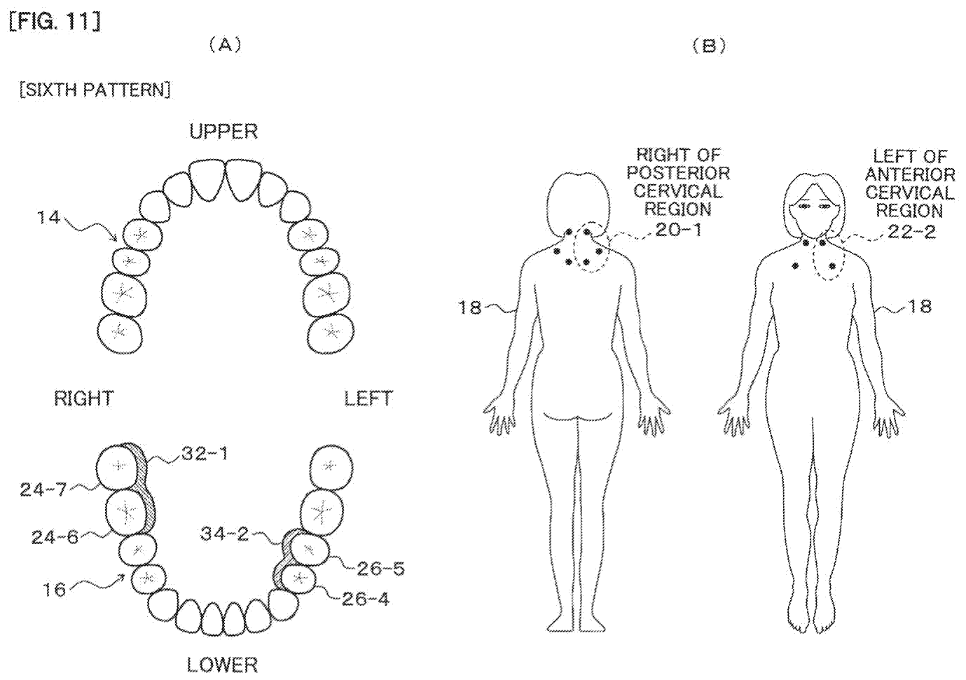

FIG. 11 is an explanatory diagram illustrating a sixth pattern in which the positions of the mandibular angle and the mental region are corrected in the occurrence of the enhancement of the basal muscle tone in the right of the posterior cervical region and the left of the anterior cervical region.

The sixth pattern shows the correction of a position in a case in which the enhancement of the basal muscle tone occurs in the right 20-1 of the posterior cervical region of the patient 18 and the left 22-2 of the anterior cervical region, as illustrated in FIG. 11(B). In this case, the wax 32-1 is applied to the lingual tooth surfaces of the first molar tooth 24-6 and second molar tooth 24-7 of the right of the mandibular teeth dentition 16 to balance laterality, as illustrated in FIG. 11(A), against the enhancement of the basal muscle tone of the right 20-1 of the posterior cervical region.

The wax 34-2 is applied to the lingual tooth surfaces of the first premolar tooth 26-4 and second premolar tooth 26-5 of the left of the mandibular teeth dentition 16 to balance laterality, as illustrated in FIG. 11(A), against the enhancement of the basal muscle tone of the left 22-2 of the anterior cervical region.

(Seventh Pattern)

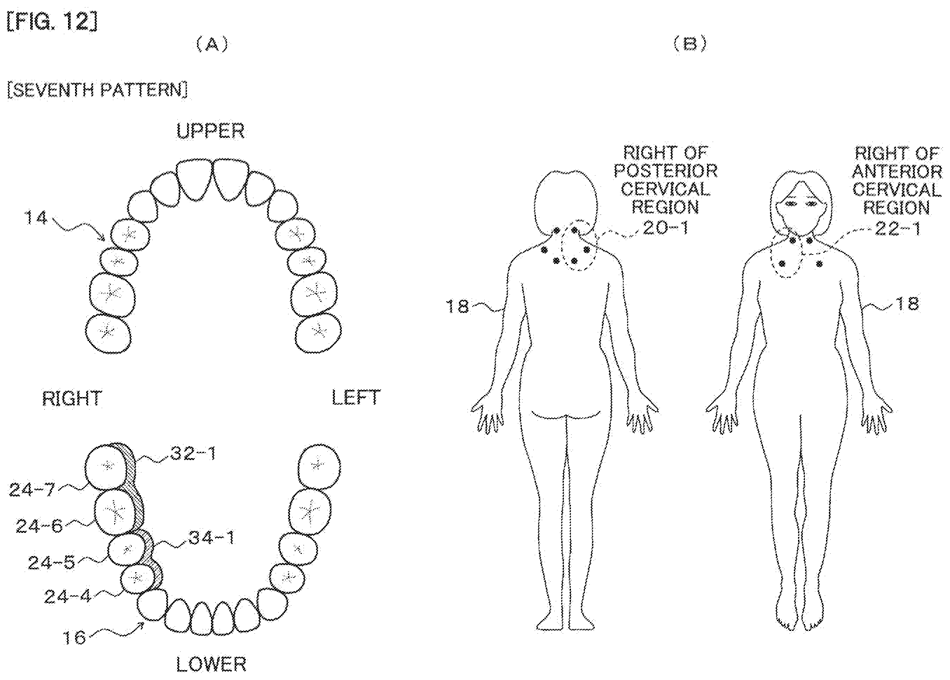

FIG. 12 is an explanatory diagram illustrating a seventh pattern in which the positions of the mandibular angle and the mental region are corrected in the occurrence of the enhancement of the basal muscle tone in the right of the posterior cervical region and the right of the anterior cervical region.

The seventh pattern shows the correction of a position in a case in which the enhancement of the basal muscle tone occurs in the right 20-1 of the posterior cervical region and the right 22-1 of the anterior cervical region of the patient 18, as illustrated in FIG. 12(B). In this case, the wax 32-1 is applied to the lingual tooth surfaces of the first molar tooth 24-6 and second molar tooth 24-7 of the right of the mandibular teeth dentition 16 to balance laterality, as illustrated in FIG. 12(A), against the enhancement of the basal muscle tone of the right 20-1 of the posterior cervical region.

In addition, the wax 34-1 is applied to the lingual tooth surfaces of the first premolar tooth 24-4 and second premolar tooth 24-5 of the right of the mandibular teeth dentition 16 to balance laterality, as illustrated in FIG. 12(A), against the enhancement of the basal muscle tone of the right 22-1 of the anterior cervical region.

(Eighth Pattern)

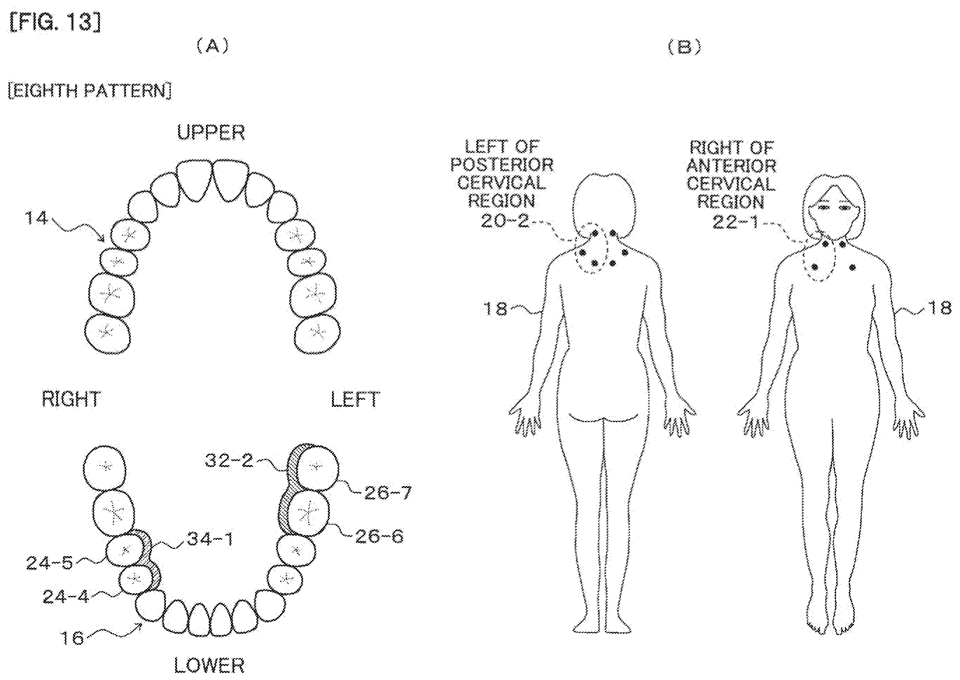

FIG. 13 is an explanatory diagram illustrating an eighth pattern in which the positions of the mandibular angle and the mental region are corrected in the occurrence of the enhancement of the basal muscle tone in the left of the posterior cervical region and the right of the anterior cervical region.

The eighth pattern shows the correction of a position in a case in which the enhancement of the basal muscle tone occurs in the left 20-2 of the posterior cervical region and the right 22-1 of the anterior cervical region of the patient 18, as illustrated in FIG. 13(B). In this case, the wax 32-2 is applied to the lingual tooth surfaces of the first molar tooth 26-6 and second molar tooth 26-7 of the left of the mandibular teeth dentition 16 to balance laterality, as illustrated in FIG. 13(A), against the enhancement of the basal muscle tone of the left 20-2 of the posterior cervical region.

In addition, the wax 34-1 is applied to the lingual tooth surfaces of the first premolar tooth 24-4 and second premolar tooth 24-5 of the right of the mandibular teeth dentition 16 to balance laterality, as illustrated in FIG. 13(A), against the enhancement of the basal muscle tone of the right 22-1 of the anterior cervical region.

(Ninth Pattern)

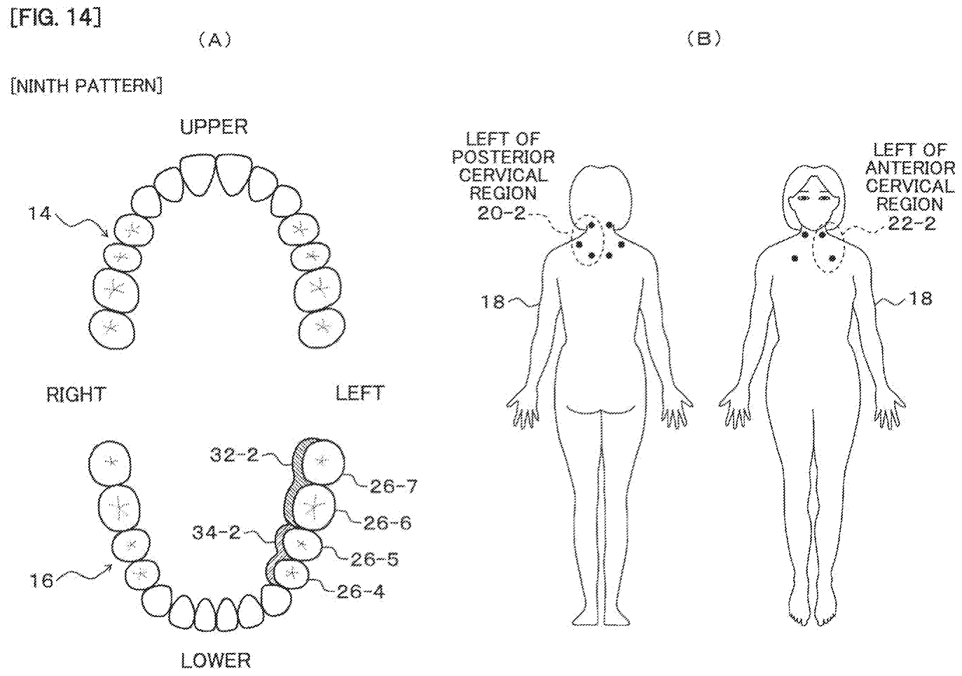

FIG. 14 is an explanatory diagram illustrating a ninth pattern in which the positions of the mandibular angle and the mental region are corrected in the occurrence of the enhancement of the basal muscle tone in the left of the posterior cervical region and the left of the anterior cervical region.

The ninth pattern shows the correction of a position in a case in which the enhancement of the basal muscle tone occurs in the left 20-2 of the posterior cervical region and the left 22-2 of the anterior cervical region of the patient 18, as illustrated in FIG. 14(B). In this case, the wax 32-2 is applied to the lingual tooth surfaces of the first molar tooth 26-6 and second molar tooth 26-7 of the left of the mandibular teeth dentition 16 to balance laterality, as illustrated in FIG. 14(A), against the enhancement of the basal muscle tone of the left 20-2 of the posterior cervical region.

In addition, the wax 34-2 is applied to the lingual tooth surfaces of the first premolar tooth 26-4 and second premolar tooth 26-5 of the left of the mandibular teeth dentition 16 to balance laterality, as illustrated in FIG. 14(A), against the enhancement of the basal muscle tone of the left 22-2 of the anterior cervical region.

[Step S4: Correction of Difference between Heights of Mandibular Bone and Mental Region]

(Procedure of Step 4)

In step S4 of FIG. 1, a chewing occlusal jaw position in which a difference between the heights of the mandibular angle and the mental region is corrected is configured.

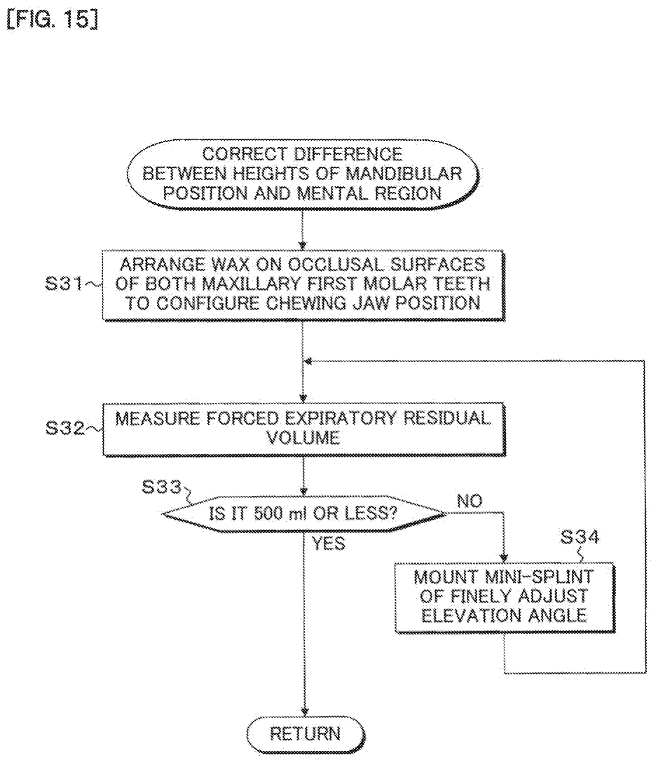

FIG. 15 is a flow diagram illustrating the details of a procedure of correcting a difference between the heights of the mandibular angle and the mental region in step S5 of FIG. 1. As illustrated in FIG. 15, in the correction of the difference between the heights of the mandibular angle and the mental region, wax is put on the occlusal surface of each of both the first molar teeth 28-6 and 30-6 in the maxillary teeth dentition 14 illustrated in FIG. 3, to configure a chewing occlusal jaw position, in step S31, and a forced expiratory residual volume is measured with a spirometer in step S32 after induction of a masseter/jaw position reflex.

A mini-splints prepared in advance is mounted on in both the first molar teeth 24-6 and 26-6 in the mandibular teeth dentition 16 to perform elevation angle correction for increasing the elevation angle of an occlusal reference plane by going to step S34 when the forced expiratory residual volume does not decrease to 500 milliliters or less in step S33, and the elevation angle correction for increasing the elevation angle is repeated by mounting the mini-splint in step S34 until the forced expiratory residual volume measured in step S32 decreases to 500 milliliters or less in step S33.

The correction of a difference between the heights of the mandibular bone and the mental region based on the elevation angle correction can be allowed to be effective in the case of a sufficient decrease in forced expiratory residual volume in a case in which the elevation angle correction is not performed even when the forced expiratory residual volume does not decrease to 500 milliliters or less even if the elevation angle the occlusal reference plane is corrected.

Herein, the forced expiratory residual volume refers to the volume of air remaining in the lung when both the expiratory muscle and the inspiratory muscle of the patient are relaxed after maximum inspiration, and is measured using a spirometer. Typically, exhalation of most air from the interior of the lung only by relaxation is considered to be ideal breathing balance.

(Mini-Splint)

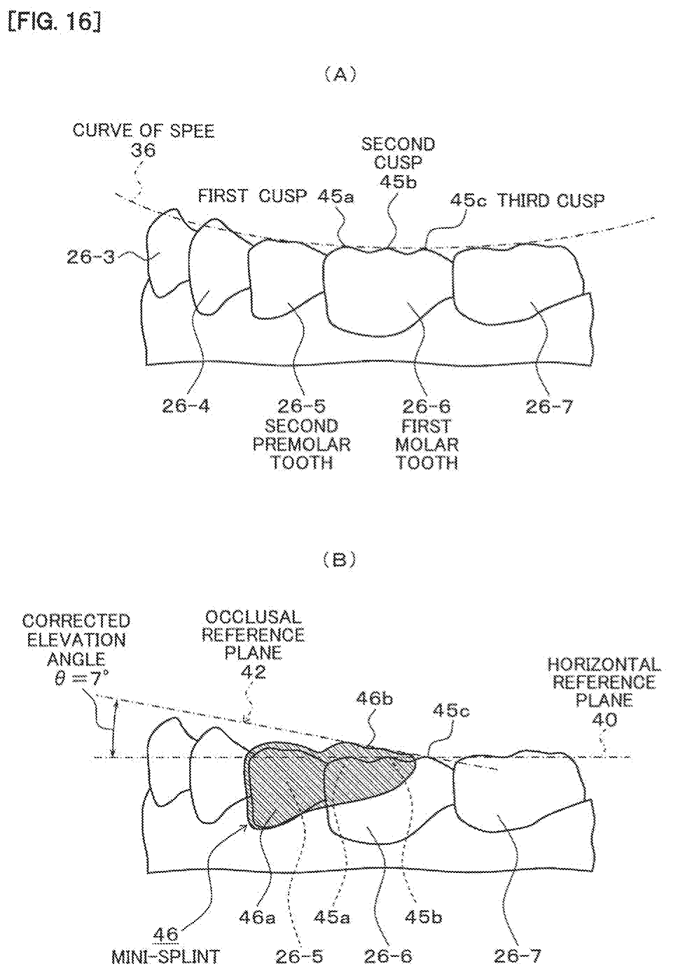

FIG. 16 is an explanatory diagram illustrating a mini-splint used for correcting a difference between the heights of the mandibular angle and the mental region. FIG. 16(A) illustrates a curve of Spee between the molar teeth and premolar teeth of the mandibular teeth dentition, and FIG. 16(B) illustrates the production of the mini-splint and the correction of an elevation angle.

The mini-splint for correcting the elevation angle of an occlusal reference plane, in which an optimal occlusal reference plane differing according to each patient is temporarily achieved, is produced in order to decrease the forced expiratory residual volume.

As illustrated in FIG. 16(A), the mandibular teeth dentition is known to have an upward curved curve 36 of Spee as a proper occlusal reference plane, but it is known that the anatomical curve 36 of Spee need not be necessarily an individually optimal occlusal reference plane. The setting of an individually optimal elevation angle based on the functional cusp of the first molar tooth 26-6 is required for allowing the occlusal reference plane of the patient to approach the individually optimal occlusal reference plane.

FIG. 16(B) illustrates production of a mini-splint having a correction elevation angle set at θ=7°. The first molar tooth 26-6 includes three buccal cusps. Assuming that the buccal cusps are a first cusp 45a, a second cusp 45b, and a third cusp 45c in order of anteriority, the second premolar tooth 26-5 is coated with a resin body having a thickness of less than 0.5 mm to form a fixation portion 46a, a portion between the first cusp 45a and second cusp 45b of the first molar tooth 26-6 is simultaneously coated, and heights from the first cusp 45a and the second cusp 45b are increased by 1 mm and 0.5 mm, respectively, with the resin body, whereby an angle-corrected surface 46b is formed to be an occlusal reference plane 42 subjected to the correction of an elevation angle of θ=7° with respect to a horizontal reference plane 40 based on the third cusp 45c.

Similarly, only a portion between a side closer to the premolar tooth and a center portion in the lingual cusp and the whole occlusal surface is also coated with a resin body so as to fit with a form of interest according to the set correction angle, and a side closer to the second molar tooth 26-7 is prevented from being coated, whereby the whole occlusal surface of the first molar tooth 26-6 is formed so that correction of a predetermined elevation angle is performed. A mini-splint 46 produced in such a manner is removed from the mandibular teeth dentition, and is prepared.

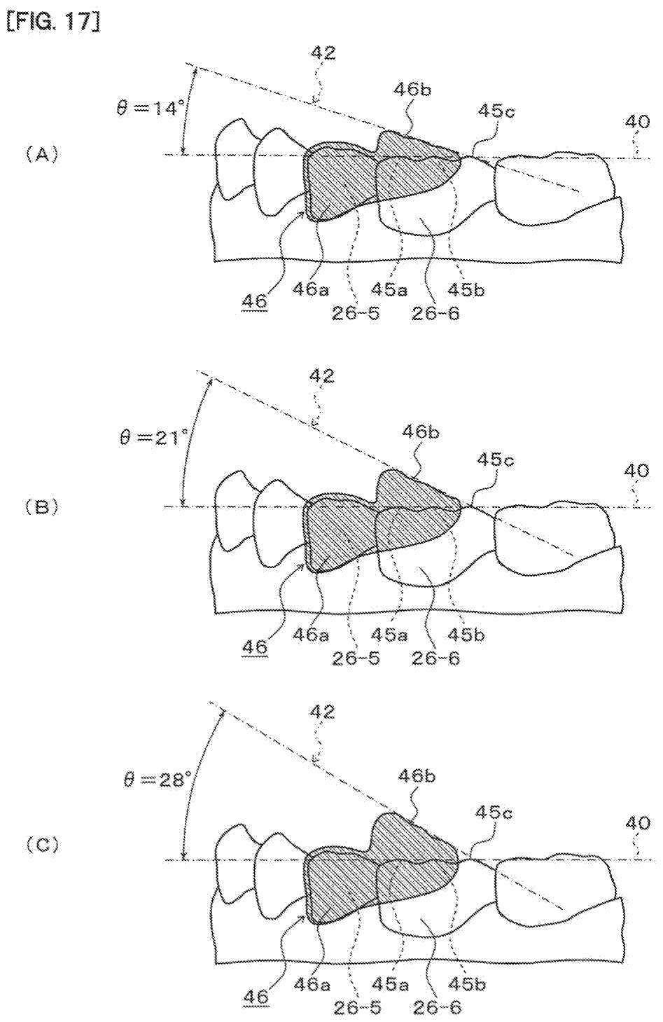

FIG. 17 is an explanatory diagram of mini-splints of which the different correction elevation angles are set. FIG. 17(A) illustrates production of a mini-splint having a correction elevation angle set at θ=14°. In the mini-splint 46, heights from the first cusp 45a and the second cusp 45b are increased by 2 mm and 1 mm, respectively, with a resin body, whereby the angle-corrected surface 46b is formed to be the occlusal reference plane 42 subjected to the correction of an elevation angle of θ=14° with respect to the horizontal reference plane 40 based on the third cusp 45c.

FIG. 17(B) illustrates production of a mini-splint having a correction elevation angle set at θ=21°. In the mini-splint 46, heights from the first cusp 45a and the second cusp 45b are increased by 3 mm and 1.5 mm, respectively, with a resin body, whereby the angle-corrected surface 46b is formed to be the occlusal reference plane 42 subjected to the correction of an elevation angle of θ=21° with respect to the horizontal reference plane 40 based on the third cusp 45c.

FIG. 17(C) illustrates production of a mini-splint having a correction elevation angle set at θ=28°. In the mini-splint 46, heights from the first cusp 45a and the second cusp 45b are increased by 4 mm and 2 mm, respectively, with a resin body, whereby the angle-corrected surface 46b is formed to be the occlusal reference plane 42 subjected to the correction of an elevation angle of θ=28° with respect to the horizontal reference plane 40 based on the third cusp 45c.

The angle θ of the elevation angle corrected in the mini-splint 46 can be set at an appropriate angle, as needed.

[Step S5: Fine Adjustment of Anteroposterior Position of Mandible]

In step S5 of FIG. 1, a chewing occlusal jaw position in which the anteroposterior position of the mandibular position is finely adjusted is configured in a state in which the mandibular angle, the mental region, and a height difference are corrected in steps S2 to S4.

In the fine adjustment of the anteroposterior position of the mandibular position in step S5, the patient finely adjusts the anteroposterior position of the mandible so that the head position of the patient in rest and relaxation is at a midline position with respect to the visual line of the patient.

When it is difficult for the patient to perform fine adjustment of the anteroposterior position, a tongue/jaw position reflex is induced to assist the fine adjustment by applying wax to the lingual tooth surface of the mandibular anterior teeth portion or the maxillary anterior teeth portion.

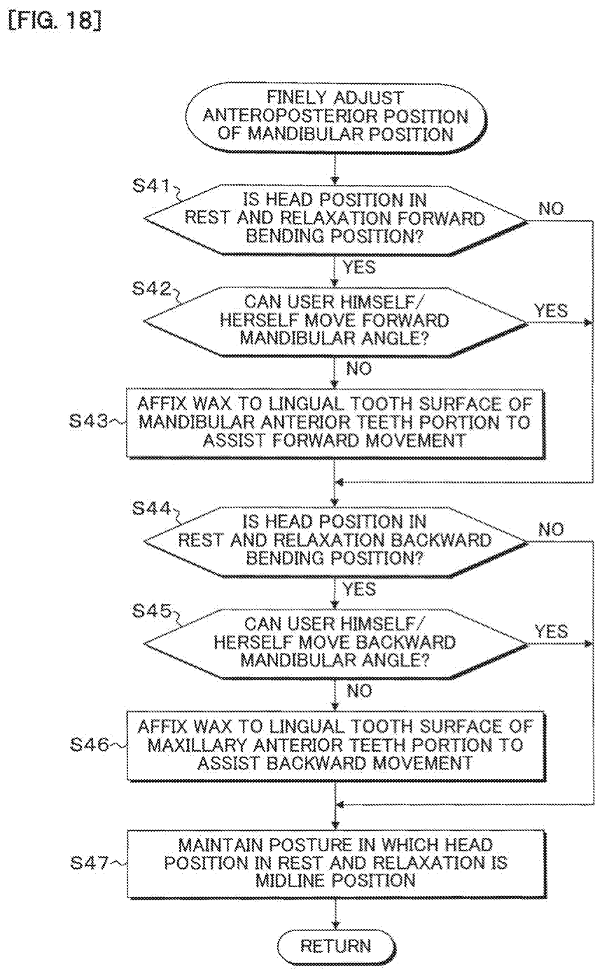

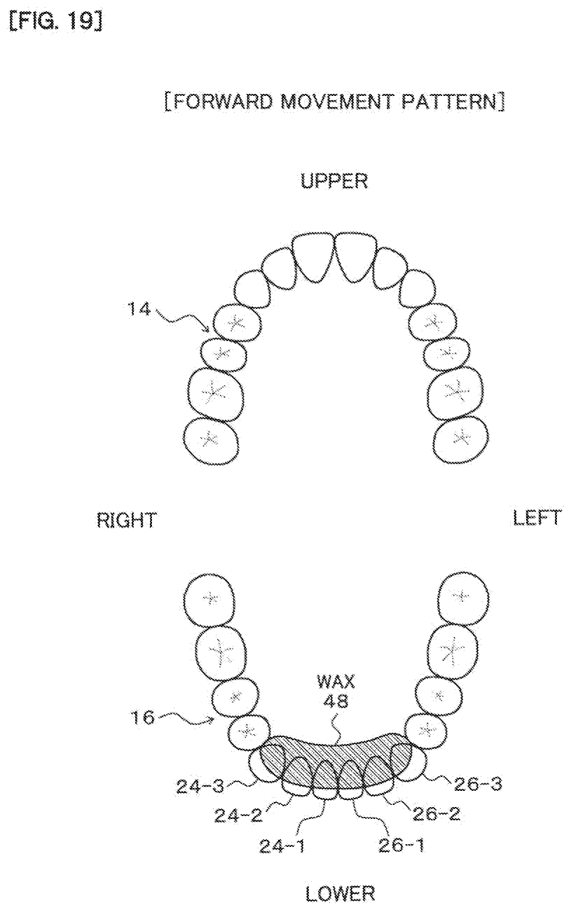

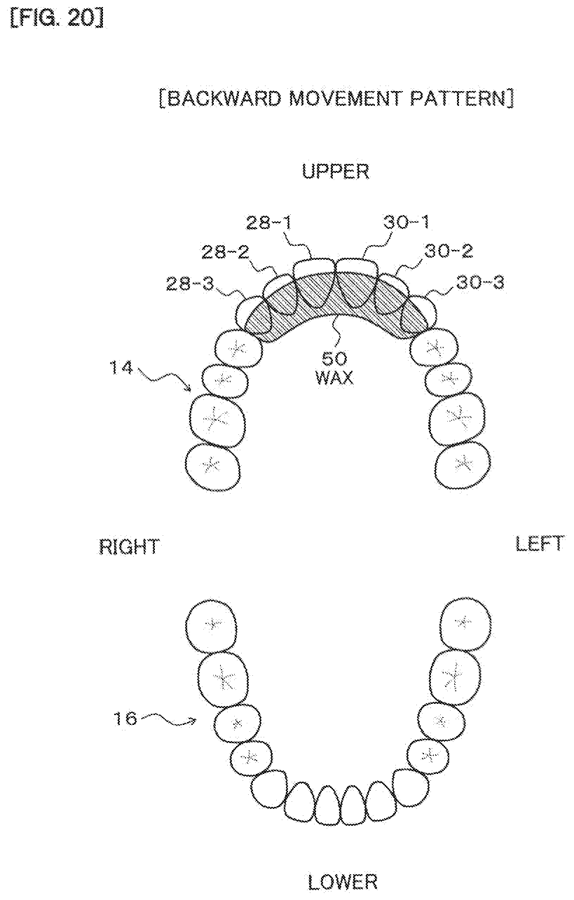

FIG. 18 is a flow diagram illustrating the details of a procedure of the anteroposterior fine adjustment of the mandibular position in step S5 of FIG. 1, FIG. 19 is an explanatory diagram illustrating a forward movement pattern in which the mandible is moved forward when the head position in rest and relaxation is bent forward, and FIG. 20 is an explanatory diagram illustrating a backward movement pattern in which the mandible is moved backward when the head position in rest and relaxation is bent backward.

As illustrated in FIG. 18, going to step S42 is performed when the head position of the patient in rest and relaxation is a forward bending position in step S41, and going to step S44 is performed when the patient can perform forward movement of the mandibular angle.

When the forward movement of the mandibular angle by the patient is not possible in step S42, going to step S43 is performed, wax 48 is applied to the lingual tooth surface of the anterior teeth portion including the central incisors 24-1 and 26-1, the lateral incisors 24-2 and 26-2, and cuspids 24-3 and 26-3 in the mandibular teeth dentition 16 as illustrated in FIG. 19, and the thickness of the wax 48 is adjusted as needed to induce a tongue/jaw position reflex to assist the forward movement of the mandibular angle by the patient.

In contrast, when the head position of the patient in rest and relaxation time is a backward bending position in step S44, going to step S45 is performed, and a posture in which the head position in the rest and relaxation is the midline position is maintained by going to step S47 when the backward movement of the mandibular angle by the patient is possible.

When the backward movement of the mandibular angle by the patient is impossible in step S45, going to step S46 is performed, wax 50 is applied to the lingual tooth surface of the anterior teeth portion including the central incisors 28-1 and 30-1, the lateral incisors 28-2 and 30-2, and the cuspids 28-3 and 30-3 in the maxillary teeth dentition 14, as illustrated in FIG. 20, and the thickness of the wax 50 is adjusted as needed to induce a tongue/jaw position reflex to assist the backward movement of the mandibular angle by the patient.

The optimized chewing occlusal state in which the systemic basal muscle tone of the patient is balanced is configured by the procedures of step S2 to S5 in FIG. 1 as described above.

[Step S6: Production of Optimum Occlusal Impression Member]

In step S6 of FIG. 1, a bite material is put between the maxillary teeth dentition and lower teeth dentition of the patient in an optimum occlusal state configured in steps S2 to S5, to produce an optimum occlusal impression member on which the impressions of the maxillary teeth dentition and the lower teeth dentition in a positional relationship in the optimum occlusal state are carved.

In the production of the optimum occlusal impression member using the bite material, it is confirmed that, for example, the lateral balance of the basal muscle tone of the posterior cervical region and the lateral balance of the basal muscle tone of the anterior cervical region are equivalent to each other, neither lumbago nor back pain occurs, and the head position in rest and relaxation is a midline position with respect to the visual line of the patient, and an operator and the patient mutually confirm that a systemic pain symptom is resolved.

[Step S7: Reproduction of Optimum Occlusal State with Occludator]

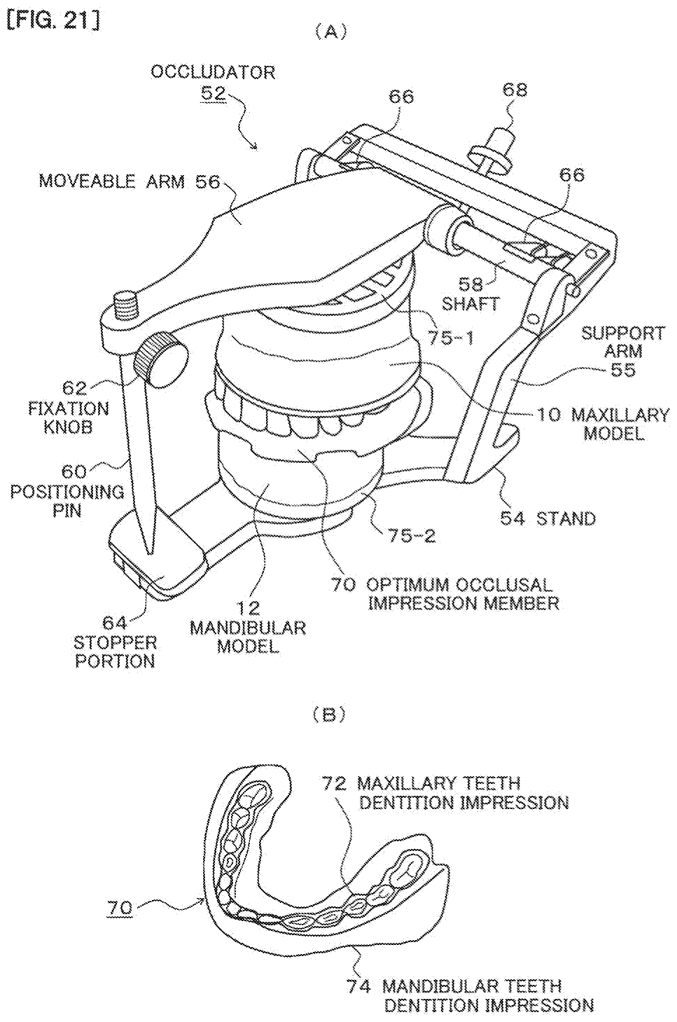

FIG. 21 is an explanatory diagram illustrating an occludator in which maxillary and mandibular models are fixed using an optimum occlusal teeth mark member taken from an optimized chewing occlusal state. FIG. 21(A) illustrates the occludator, and FIG. 21(B) illustrates the optimum occlusal impression member taken out. The occludator is an example, and an appropriate occludator can be used as the occludator.

As illustrated in FIG. 21(A), in an occludator 52, a moveable arm 56 is rotatably disposed, through a shaft 58, on a support arm 55 standing behind a stand 54, a positioning pin 60 is disposed on a leading end of the moveable arm 56, and a leading end of the positioning pin 60 abuts on a stopper portion 64 arranged on a leading end of the stand 54. The length of the positioning pin 60 from the moveable arm 56 can be adjusted, and is fixed to a length adjusted by a fixation knob 62.

A spring 66 is arranged between the shaft 58 and a side of the rear of the support arm 55, and biases the moveable arm 56 in a forward rolling manner. In addition, an adjustment knob 68 is disposed on a side of the rear of the support arm 55. When the length of the positioning pin 60 is adjusted, a leading end of the pin is enabled to rise by pushing a screw shaft, integrally disposed on the adjustment knob 68, on the rear of the moveable arm 56.

As illustrated in FIG. 21(B), a maxillary teeth dentition impression 72 in an optimized chewing occlusal state is carved on the upper side of an optimum occlusal impression member 70 produced in step S6 of FIG. 1, and a mandibular teeth dentition impression 74 in an optimized chewing occlusal state is carved on the upper side.

In the occludator 52, an optimized chewing occlusal state is generated by arranging the optimum occlusal impression member 70 between the maxillary model 10 and the mandibular model 12 produced in step S1 and illustrated in FIG. 3, the maxillary model 10 is fixed to a fixation member 75-1 in the lower side of the moveable arm 56 using gypsum in this state, the mandibular model 12 is fixed to a fixation member 75-2 on the stand 54 using gypsum to reproduce the optimized chewing occlusal state, and the leading end of the positioning pin 60 hits the stopper portion 64 in this state to fix the positioning pin 60, by the fixation knob 62, at a position at which the moveable arm 56 is supported.

Subsequently, the moveable arm 56 is upward rotated and opened, and the optimum occlusal impression member 70 arranged between the maxillary model 10 and the mandibular model 12 is removed, whereby the optimum occlusal state configured in steps S2 to S5 of FIG. 1 is reproduced as the positional relationship between the maxillary model 10 and the mandibular model 12 fixed to the occludator 52.

[Step S8: Production of Mouthpiece]

In step S8 of FIG. 1, a mouthpiece mounted on the maxillary teeth dentition of the patient is produced using the occludator in which the maxillary model 10 and the mandibular model 12 are fixed in the positional relationship in the optimized chewing occlusal state in step S7, and which is illustrated in FIG. 21.

(Production of Mouthpiece Part Using Maxillary Model)

First, a mouthpiece part is produced using the maxillary model produced in step S1. The mouthpiece part is produced using, for example, a vacuum former known as a known mouthpiece former.

The maxillary model and a soft resin sheet (ethylene-vinyl acetate copolymer: EVA) having a uniform thickness of around 2 to 3 mm are set in the vacuum former, and are heated and vacuum-sucked.

As a result, the heated soft resin sheet is deformed to fit with the outer shape of the maxillary teeth dentition and the gingival portion in the maxillary model by vacuum-sucking the soft resin sheet, to form a mouthpiece prototype.

Subsequently, the mouthpiece prototype is separated from the soft resin sheet, thereby producing a mouthpiece part on which the maxillary model is carved.

(Formation of Impression of Mandibular Teeth Dentition Using Occludator)

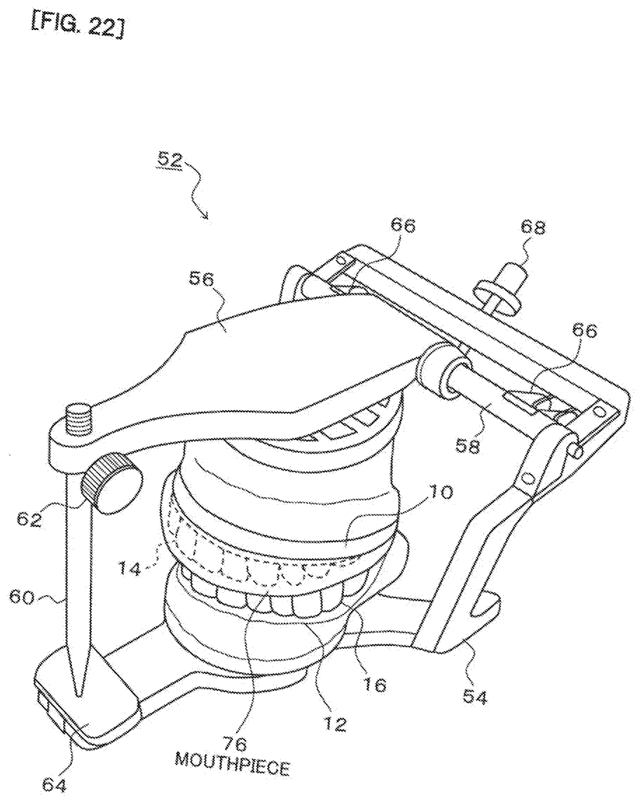

FIG. 22 is an explanatory diagram illustrating production of a mouthpiece according to the present invention using the occludator illustrated in FIG. 21.

The mouthpiece part on which the maxillary model is carved is mounted on the maxillary model 10 of the occludator 52 in which the positional relationship of the optimum occlusal state of the maxillary model 10 and the mandibular model 12 illustrated in FIG. 21 is reproduced, the occlusal surface as the bottom surface of the mouthpiece part is heated and softened, the occludator 52 is closed, as illustrated in FIG. 22, in this state, and the maxillary model 10 and the mandibular model 12 on which the mouthpiece part is mounted are occluded. The mouthpiece hart is cooled and cured, followed by opening the occludator 52, whereby an impression on which the teeth dentition of the mandibular model 12 in the positional relationship in the optimum occlusal state is carved is formed on the occlusal surface (undersurface) of the mouthpiece part to complete a mouthpiece 76.

(Mouthpiece)

FIG. 23 is an explanatory diagram illustrating the plane, side surface, and bottom surface of the mouthpiece according to the present invention produced using the occludator of FIG. 22.

As illustrated in FIG. 23, in the mouthpiece 76 produced in such a manner, a maxillary teeth dentition impression 82 caused by the maxillary model 10 is carved on a bottom in an upper opening 80 of a mouthpiece body 78, a mandibular teeth dentition impression 84 of the mandibular model 12 is carved on the bottom surface of the mouthpiece body 78, and an optimized occlusal positional relationship, which is primarily in a chewing occlusal state, is reproduced between the maxillary teeth dentition impression 82 and the mandibular teeth dentition impression 84.

Therefore, when the mouthpiece 76 is mounted on the maxillary teeth dentition of the patient to achieve the chewing occlusal state with the mandibular teeth dentition, the mandibular teeth dentition of the patient is occluded and positioned on the mandibular teeth dentition impression 84 on the undersurface of the mouthpiece 76, whereby the chewing occlusal jaw position is allowed to be adequate so that the mandible is at a proper position. Thus, the lateral balance of the basal muscle tone of the posterior cervical region and the lateral balance of the basal muscle tone of the anterior cervical region are allowed to be equivalent to each other, and the systemic basal muscle tone is balanced. As a result, a systemic pain symptom caused by fibromyalgia can be basically resolved.

The mouthpiece in this stage is in a state in which allowing of the resting jaw position to be adequate is unfinished although the chewing occlusal jaw position is allowed to be adequate.

In the present embodiment, a case is described in which the mouthpiece is produced in two processes of a process of producing the mouthpiece part using the mouthpiece former and a process of impressing the mandibular teeth dentition on the mouthpiece part using the occludator. However, use of a mouthpiece former having the function of the occludator enables the mouthpiece to be produced in one process.

[Step S9: Fine Adjustment of Mouthpiece]

In step S9 of FIG. 1, the mouthpiece produced in step S8 is mounted on the maxillary teeth dentition of the patient, and is finely adjusted.

In the fine adjustment of the mouthpiece in step S8, the fine adjustment for obtaining an optimal resting jaw position is performedby touching the muscle of the patient in a state in which the mouthpiece is mounted on the patient. In the fine adjustment of the mouthpiece, fine adjustment corresponding to the mandibular angle, fine adjustment corresponding to the mental region, and, in addition, fine adjustment of the anteroposterior position of the mandibular position are performed.

The fine adjustment corresponding to the mandibular angle and the mental region is performed to reduce, by polishing, the thickness of the lingual surface of the mouthpiece located at a molar tooth closer to a side, in which tension is enhanced, when laterality is observed in the basal muscle tone of the posterior cervical region. When laterality is observed in the basal muscle tone of the anterior cervical region of the patient, the adjustment is performed by reducing, by polishing, the thickness of the lingual surface of the mouthpiece located at a premolar tooth closer to a side in which tension is enhanced.

The fine adjustment of the anteroposterior position of the mandibular position is performed by reducing, by polishing, the thickness of the lingual tooth surface of the anterior teeth side of the mouthpiece when the head position is bent forward. When the head position is bent backward, the adjustment is performed to increase, by thickening, the thickness of the lingual tooth surface of the anterior teeth side of the uthpiece.

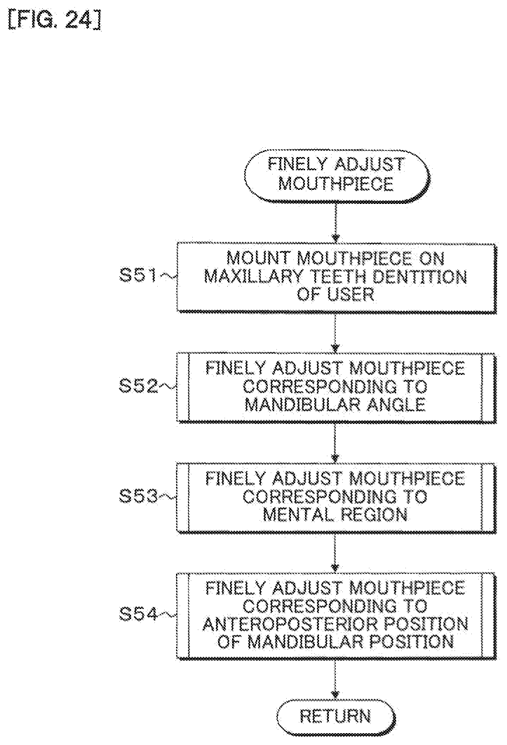

FIG. 24 is a flow diagram illustrating a procedure of finely adjusting the mouthpiece illustrated in FIG. 23. As illustrated in FIG. 24, in the fine adjustment of the mouthpiece, the mouthpiece is mounted on the maxillary teeth dentition of the patient to achieve a resting jaw position in step S51, fine adjustment of the mouthpiece corresponding to the mandibular angle is performed in step S52, fine adjustment of the mouthpiece corresponding to the mental region is performed in step S53, and fine adjustment of the mouthpiece corresponding to the anteroposterior position of the mandibular position is performed in step S54.

[Step S52: Fine Adjustment of Mouthpiece Corresponding to Mandibular Angle]

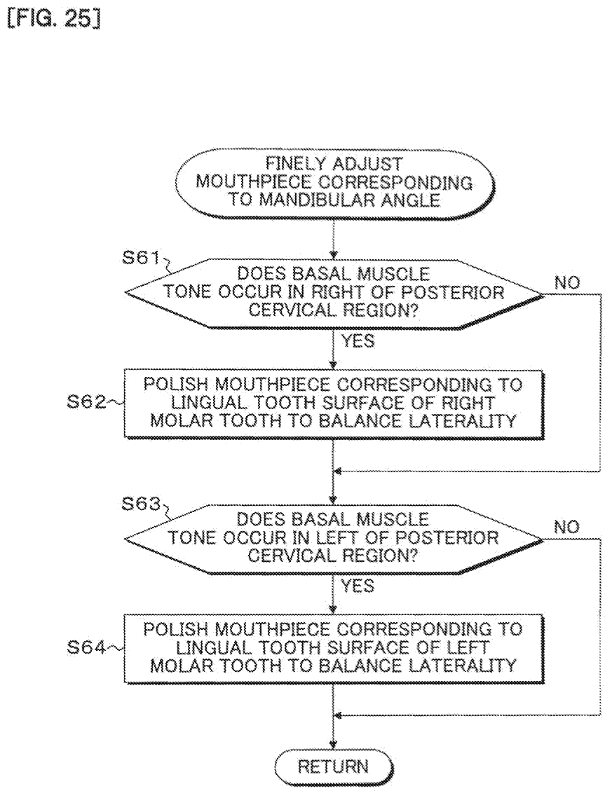

FIG. 25 is a flow diagram illustrating the details of the fine adjustment of the mouthpiece corresponding to the mandibular angle in step S52 of FIG. 24. In the fine adjustment of the mouthpiece corresponding to the mandibular angle in FIG. 25, when the enhancement of the basal muscle tone occurs in the right of the posterior cervical region of the patient in step S61, going to step S62 is performed, and the thickness of the mouthpiece located on the lingual tooth surface of a maxillary right molar tooth is reduced by polishing in a unit of micron order to balance laterality.

When the enhancement of the basal muscle tone occurs in the left of the posterior cervical region of the patient in step S63, going to step S64 is performed, and the thickness of the mouthpiece located on the lingual tooth surface of a maxillary left molar tooth is reduced by polishing in a unit of micron order to balance laterality.

The adjustment of the balance of the mouthpiece, located on the lingual tooth surface, by polishing is not performed when the enhancement of the basal muscle tone does not occur in the right of the posterior cervical region of the patient instep S61, and the enhancement of the basal muscle tone does not occur in the left of the posterior cervical region of the patient in step S63.

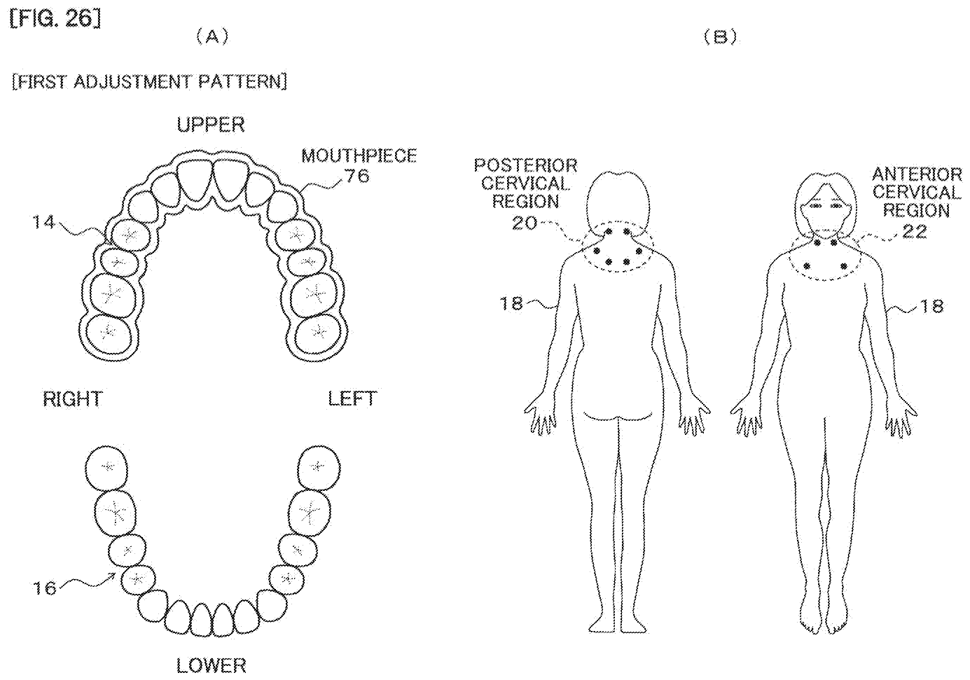

The fine adjustment of the position of the mandibular angle in FIG. 25 can be divided into a first fine adjustment pattern to a third fine adjustment pattern, illustrated in FIG. 26 to FIG. 28.

(First Fine Adjustment Pattern)

FIG. 26 is an explanatory diagram illustrating a first fine adjustment pattern without the laterality of the basal muscle tones of the posterior cervical region and the anterior cervical region and without the fine adjustment of a mouthpiece. FIG. 26(A) illustrates the maxillary teeth dentition and the mandibular teeth dentition, and FIG. 26(B) illustrates sites in which the enhancement of the basal muscle tones of the posterior and anterior cervical regions of the patient occurs. The mouthpiece 76 in FIG. 26(A) is illustrated in a cross section in the case of being mounted on the maxillary teeth dentition.

The first fine adjustment pattern is a case in which each of the posterior cervical region 20 and anterior cervical region 22 of the patient 18 is balanced without laterality as illustrated in FIG. 26(B). In this case, the adjustment of the balance by polishing the mouthpiece is not performed as illustrated in FIG. 26(A).

(Second Fine Adjustment Pattern)

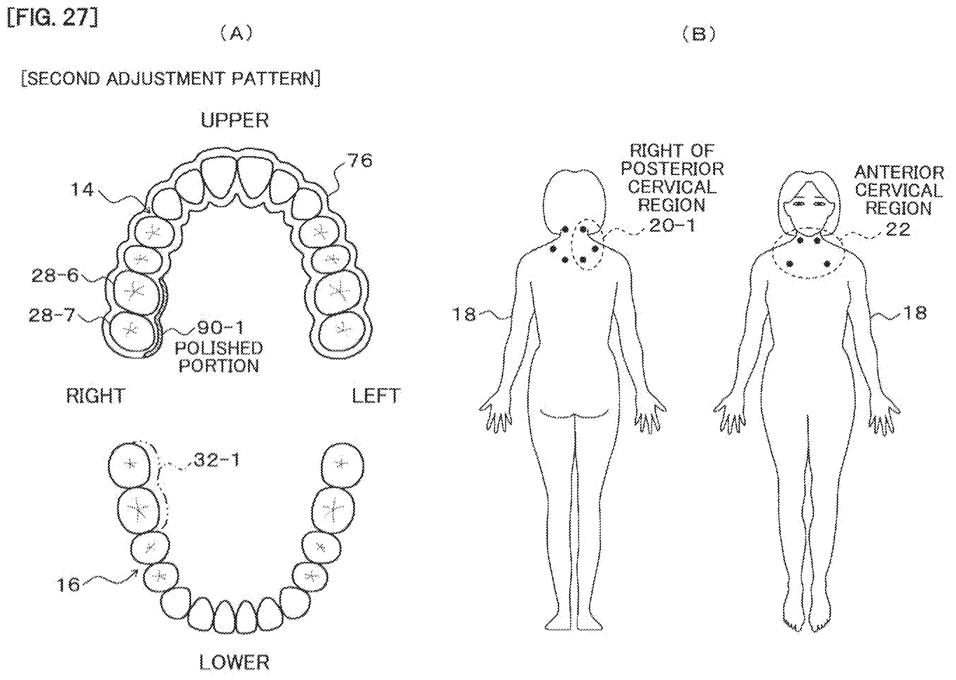

FIG. 27 is an explanatory diagram illustrating a second fine adjustment pattern in which a mouthpiece is finely adjusted in the occurrence of the enhancement of the basal muscle tone in the right of the posterior cervical region.

The second fine adjustment pattern shows the fine adjustment of the mouthpiece 76 in a case in which the enhancement of the basal muscle tone occurs in the right 20-1 of the posterior cervical region of the patient 18, and the anterior cervical region 22 of the patient 18 is balanced without laterality, as illustrated in FIG. 27(B). In this case, as illustrated in FIG. 27(A), a polished portion 90-1 in which the thickness of the mouthpiece 76 located on the lingual tooth surfaces of the first molar tooth 28-6 and second molar tooth 28-7 of the right of the maxillary teeth dentition 14 is reduced by polishing is formed to balance laterality.

(Third Fine Adjustment Pattern)

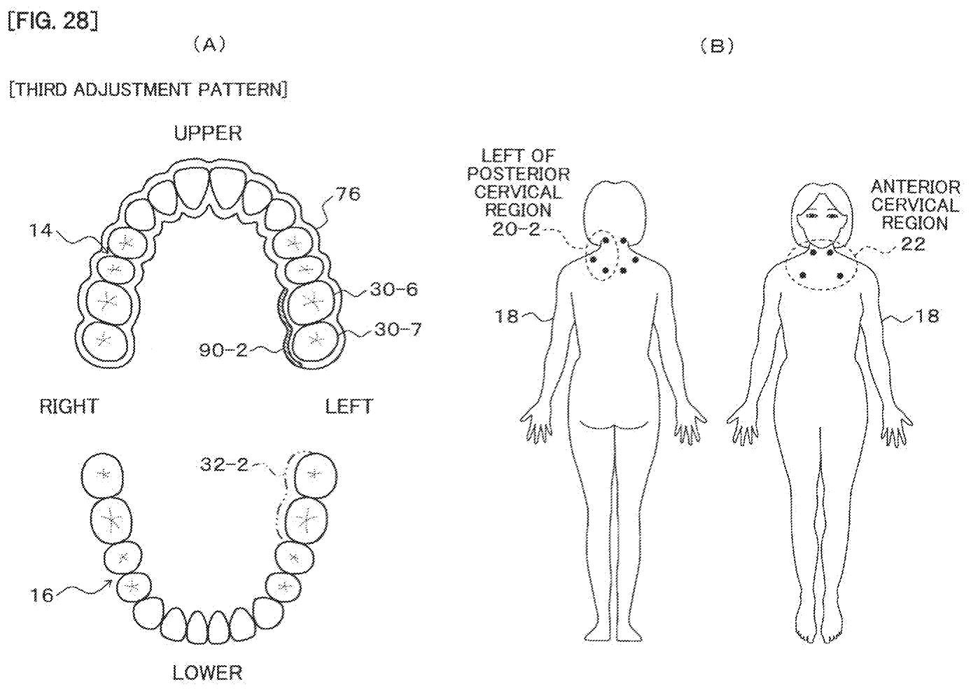

FIG. 28 is an explanatory diagram illustrating a third fine adjustment pattern in which a mouthpiece is finely adjusted in the occurrence of the enhancement of the basal muscle tone in the left of the posterior cervical region.

The third fine adjustment pattern shows the fine adjustment of the mouthpiece 76 in a case in which the basal muscle tone occurs in the left 20-2 of the posterior cervical region of the patient 18, and the anterior cervical region 22 of the patient 18 is balanced without laterality, as illustrated in FIG. 28(B). In this case, as illustrated in FIG. 28(A), a polished portion 90-2 in which the thickness of the mouthpiece 76 located on the lingual tooth surfaces of the first molar tooth 30-6 and second molar tooth 30-7 of the left of the maxillary teeth dentition 14 is reduced by polishing is formed to balance laterality.

[Step S53: Fine Adjustment of Mouthpiece Corresponding to Mental Region]

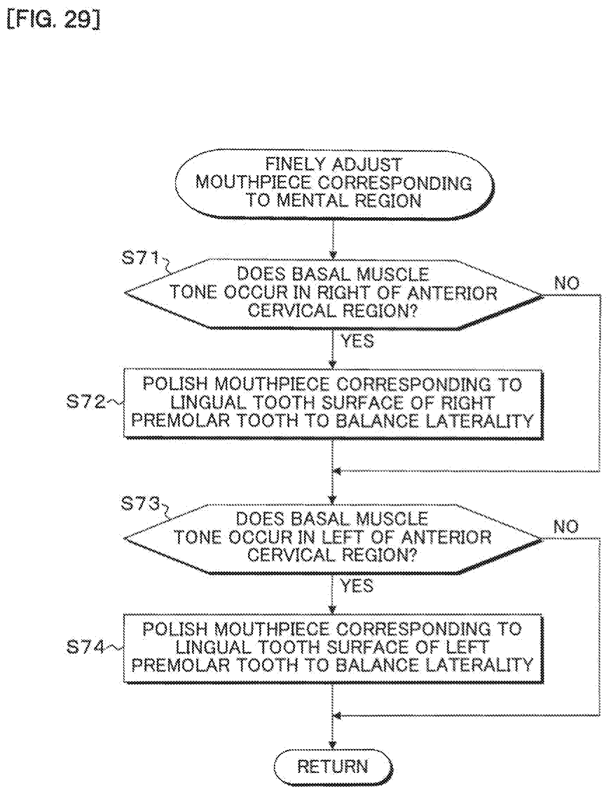

FIG. 29 is a flow diagram illustrating the details of the fine adjustment of a mouthpiece corresponding to the mental region in step S53 of FIG. 24.

In the fine adjustment of the mouthpiece corresponding to the mental region illustrated in FIG. 29, the thickness of the mouthpiece located on the lingual tooth surface of a maxillary right premolar tooth is reduced by polishing in a unit of micron order to balance laterality by going to step S72 when the enhancement of the basal muscle tone occurs in the right of the anterior cervical region of the patient in step S71.

The thickness of the mouthpiece located on the lingual tooth surface of a maxillary left premolar tooth is reduced by polishing in a unit of micron order to balance laterality by going to step S74 when the enhancement of the basal muscle tone occurs in the left of the anterior cervical region of the patient in step S73.

The fine adjustment of the mouthpiece by polishing is not performed when the enhancement of the basal muscle tone does not occur in the right of the anterior cervical region of the patient in step S71, and the enhancement of the basal muscle tone does not occur in the left of the anterior cervical region of the patient in step S73.

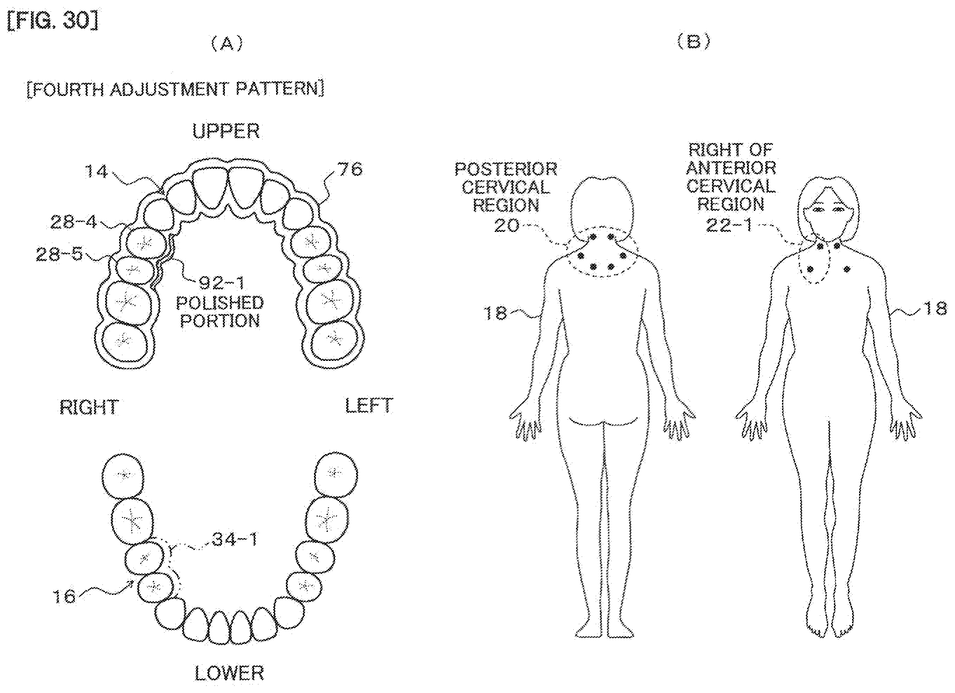

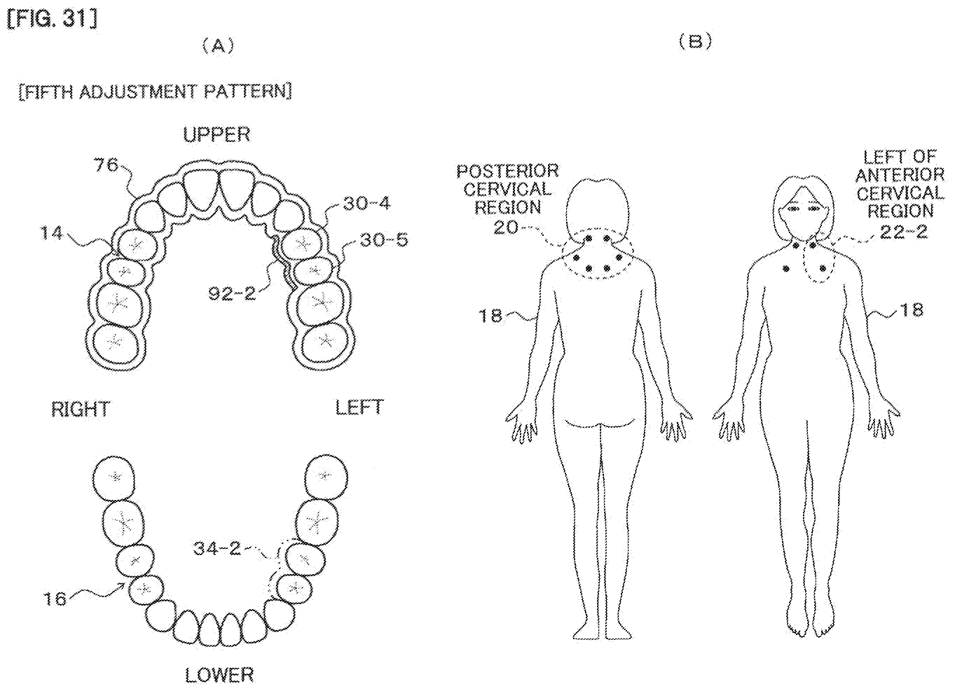

The fine adjustment of the mouthpiece corresponding to the mandibular angle in FIG. 29 can be divided into a fourth fine adjustment pattern and a fifth fine adjustment pattern, illustrated in FIG. 30 and FIG. 31.

(Fourth Fine Adjustment Pattern)

FIG. 30 is an explanatory diagram illustrating a fourth fine adjustment pattern in which a mouthpiece is finely adjusted in the occurrence of the enhancement of the basal muscle tone in the right of the anterior cervical region.

The fourth fine adjustment pattern shows the fine adjustment of the mouthpiece 76 in a case in which the enhancement of the basal muscle tone occurs in the right 22-1 of the anterior cervical region of the patient 18, and the posterior cervical region 20 of the patient 18 is balanced without laterality, as illustrated in FIG. 30(B). In this case, as illustrated in FIG. 30(A), a polished portion 92-1 in which the thickness of the mouthpiece 76 located on the lingual tooth surfaces of the first premolar tooth 24-4 and second premolar tooth 24-5 of the right of the maxillary teeth dentition is reduced by polishing is formed to balance laterality.

(Fifth Fine Adjustment Pattern)

FIG. 31 is an explanatory diagram illustrating a fifth fine adjustment pattern in which a mouthpiece is finely adjusted in the occurrence of the enhancement of the basal muscle tone in the left of the anterior cervical region.

The fifth fine adjustment pattern shows the fine adjustment of the mouthpiece 76 in a case in which the enhancement of the basal muscle tone occurs in the left 22-2 of the anterior cervical region of the patient 18, and the posterior cervical region 20 of the patient 18 is balanced without laterality, as illustrated in FIG. 31(B). In this case, as illustrated in FIG. 31(A), a polished portion 92-2 in which the thickness of the mouthpiece 76 located on the lingual tooth surfaces of the first premolar tooth 30-4 and second premolar tooth 30-5 of the left of the maxillary teeth dentition is reduced by polishing is formed to balance laterality.

[Fine Adjustment of Mouthpiece in Combination of Mandibular Angle and Mental Region]

Examples of the fine adjustment of the mouthpiece corresponding to both of the mandibular angle and the mental region in FIG. 24 include the following sixth fine adjustment pattern to ninth fine adjustment pattern.

(Sixth Fine Adjustment Pattern)

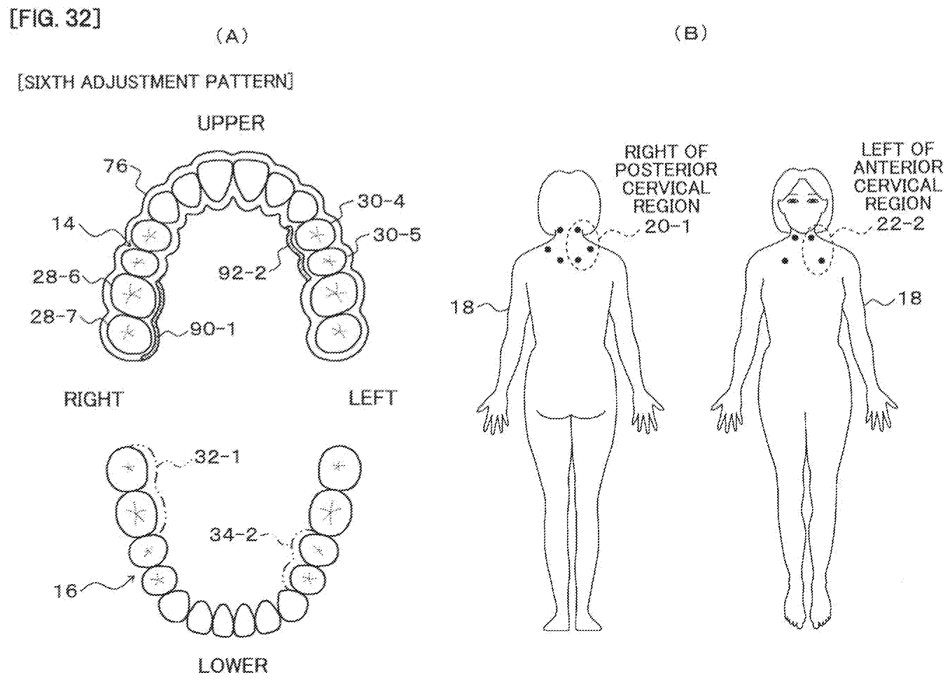

FIG. 32 is an explanatory diagram illustrating a sixth fine adjustment pattern in which a mouthpiece is finely adjusted in the occurrence of the enhancement of the basal muscle tone in the right of the posterior cervical region and the left of the anterior cervical region.

The sixth fine adjustment pattern shows the fine adjustment of the mouthpiece 76 in a case in which the enhancement of the basal muscle tone occurs in the right 20-1 of the posterior cervical region and the left 22-2 of the anterior cervical region of the patient 18, as illustrated in FIG. 32(B). In this case, the polished portion 90-1 in which the thickness of the mouthpiece 76 located on the lingual tooth surfaces of the first molar tooth 28-6 and the second molar tooth 28-7 of the right of the maxillary teeth dentition 14 are reduced by polishing is formed to balance laterality, as illustrated in FIG. 32(A), against the enhancement of the basal muscle tone of the right 20-1 of the posterior cervical region.

As illustrated in FIG. 32(A), the polished portion 92-2 in which the thickness of the mouthpiece 76 located on the lingual tooth surfaces of the first premolar tooth 30-4 and the second premolar tooth 30-5 of the left of the maxillary teeth dentition 14 is reduced by polishing is formed to balance laterality against the enhancement of the basal muscle tone of the left 22-2 of the anterior cervical region.

(Seventh Fine Adjustment Pattern)

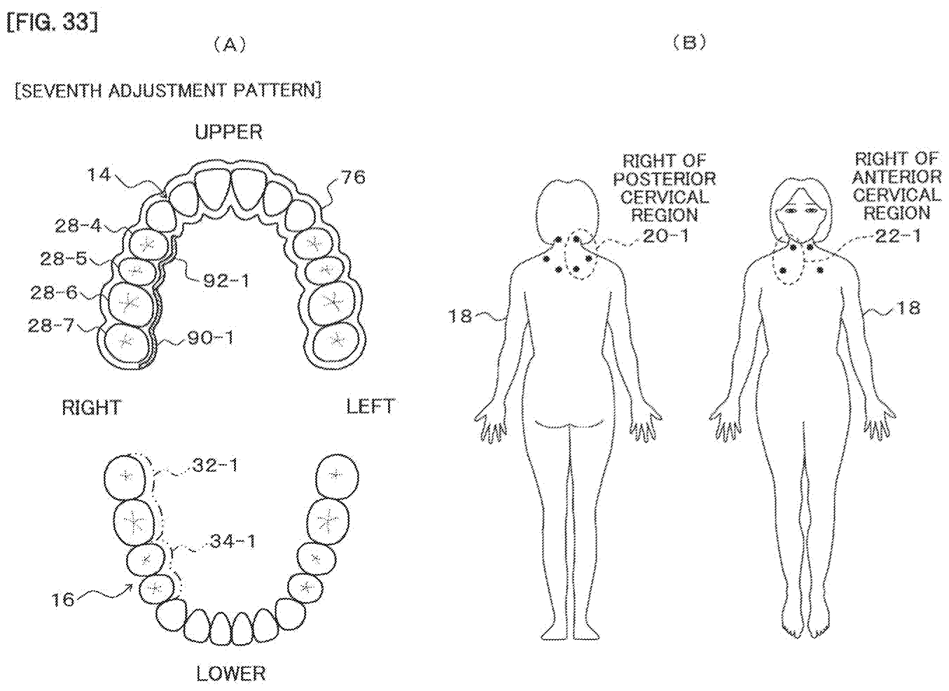

FIG. 33 is an explanatory diagram illustrating a seventh fine adjustment pattern in which a mouthpiece is finely adjusted in the occurrence of the enhancement of the basal muscle tone in the right of the posterior cervical region and the right of the anterior cervical region.

The seventh fine adjustment pattern shows the fine adjustment of the mouthpiece 76 in a case in which the enhancement of the basal muscle tone occurs in the right 20-1 of the posterior cervical region and the right 22-1 of the anterior cervical region of the patient 18, as illustrated in FIG. 33(B). In this case, the polished portion 92-1 in which the thickness of the mouthpiece 76 located on the lingual tooth surfaces of the first molar tooth 28-6 and the second molar tooth 28-7 of the right of the maxillary teeth dentition 14 are reduced by polishing is formed to balance laterality, as illustrated in FIG. 33(A), against the enhancement of the basal muscle tone of the right 20-1 of the posterior cervical region.

As illustrated in FIG. 33(A), the polished portion 92-1 in which the thickness of the mouthpiece 76 located on the lingual tooth surfaces of the first premolar tooth 28-4 and the second premolar tooth 28-5 of the right of the maxillary teeth dentition 14 is reduced by polishing is formed to balance laterality against the basal muscle tone of the right 22-1 of the anterior cervical region.

(Eighth Fine Adjustment Pattern)

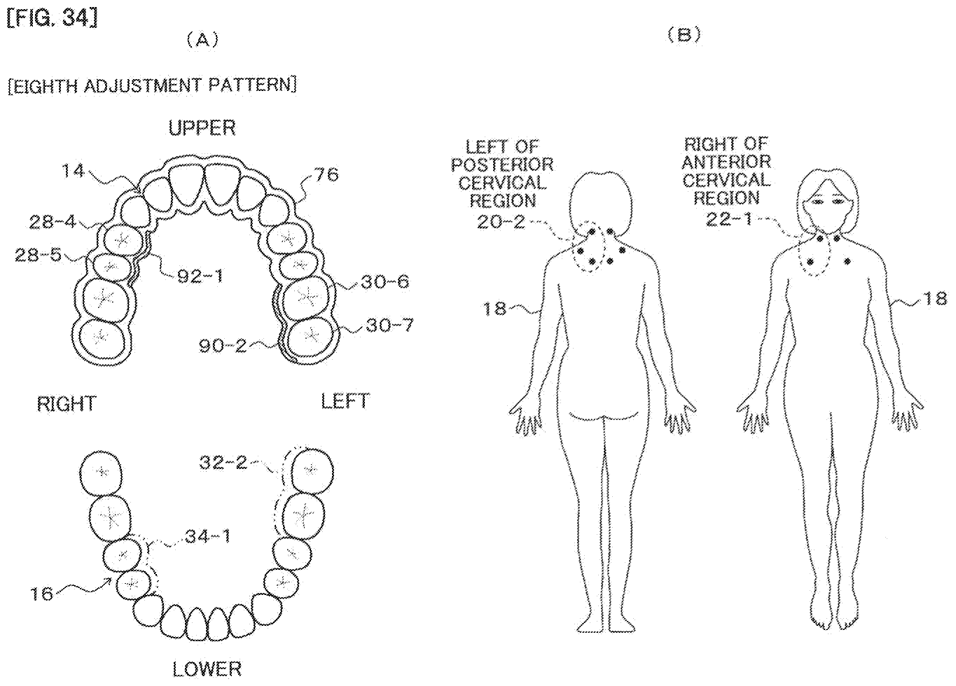

FIG. 34 is an explanatory diagram illustrating an eighth fine adjustment pattern in which a mouthpiece is finely adjusted in the occurrence of the enhancement of the basal muscle tone in the left of the posterior cervical region and the right of the anterior cervical region.

The eighth fine adjustment pattern shows the fine adjustment of the mouthpiece 76 in a case in which the enhancement of the basal muscle tone occurs in the left 20-2 of the posterior cervical region and the right 22-1 of the anterior cervical region of the patient 18, as illustrated in FIG. 34(B). In this case, the polished portion 90-2 in which the thickness of the mouthpiece 76 located on the lingual tooth surfaces of the first molar tooth 30-6 and the second molar tooth 30-7 of the left of the maxillary teeth dentition 14 are reduced by polishing is formed to balance laterality, as illustrated in FIG. 34(A), against the enhancement of the basal muscle tone of the left 20-2 of the posterior cervical region.

As illustrated in FIG. 34(A), the polished portion 92-1 in which the thickness of the mouthpiece 76 located on the lingual tooth surfaces of the first premolar tooth 28-4 and the second premolar tooth 28-5 of the right of the maxillary teeth dentition 14 is reduced by polishing is formed to balance laterality against the enhancement of the basal muscle tone of the right 22-1 of the anterior cervical region.

(Ninth Fine Adjustment Pattern)

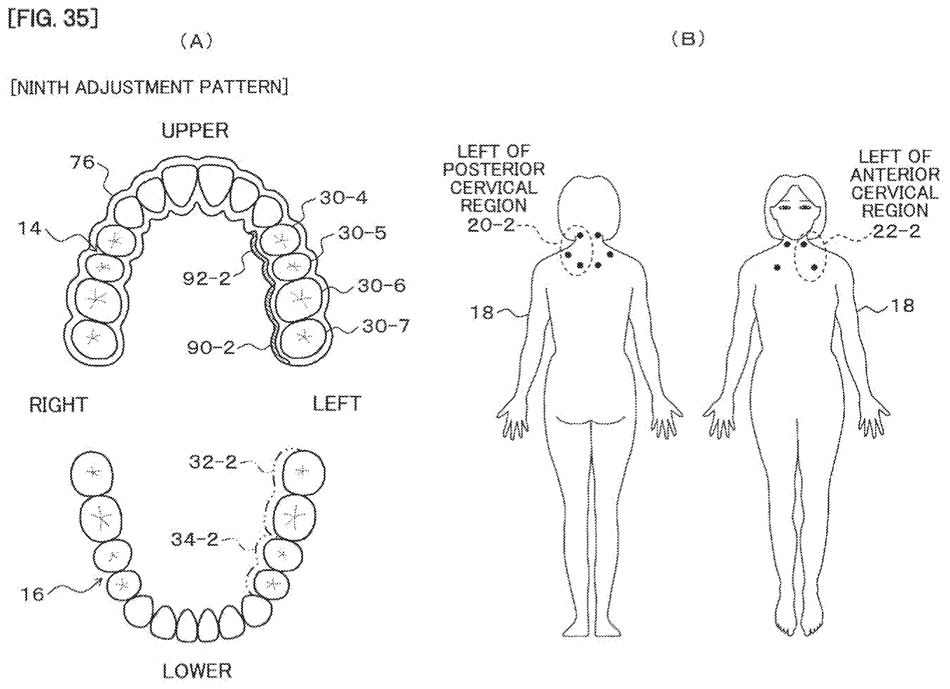

FIG. 35 is an explanatory diagram illustrating a ninth fine adjustment pattern in which a mouthpiece is finely adjusted in the occurrence of the enhancement of the basal muscle tone in the left of the posterior cervical region and the left of the anterior cervical region.

The ninth fine adjustment pattern shows the fine adjustment of the mouthpiece 76 in a case in which the enhancement of the basal muscle tone occurs in the left 20-2 of the posterior cervical region and the left 22-2 of the anterior cervical region of the patient 18, as illustrated in FIG. 35(B). In this case, the polished portion 90-2 in which the thickness of the mouthpiece 76 located on the lingual tooth surfaces of the first molar tooth 30-6 and the second molar tooth 30-7 of the left of the maxillary teeth dentition 14 are reduced by polishing is formed to balance laterality, as illustrated in FIG. 35(A), against the enhancement of the basal muscle tone of the left 20-2 of the posterior cervical region.

As illustrated in FIG. 35(A), the polished portion 92-2 in which the thickness of the mouthpiece 76 located on the lingual tooth surfaces of the first premolar tooth 30-4 and the second premolar tooth 30-5 of the left of the maxillary teeth dentition 14 is reduced by polishing is formed to balance laterality against the enhancement of the basal muscle tone of the left 22-2 of the anterior cervical region.

[Step 54: Fine Adjustment of Mouthpiece Corresponding to Anteroposterior Position of Mandible]

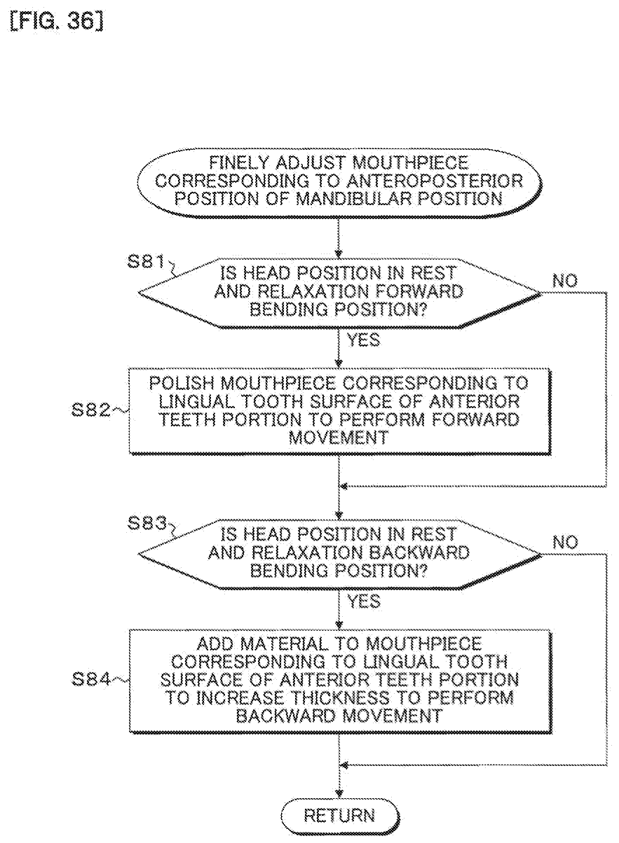

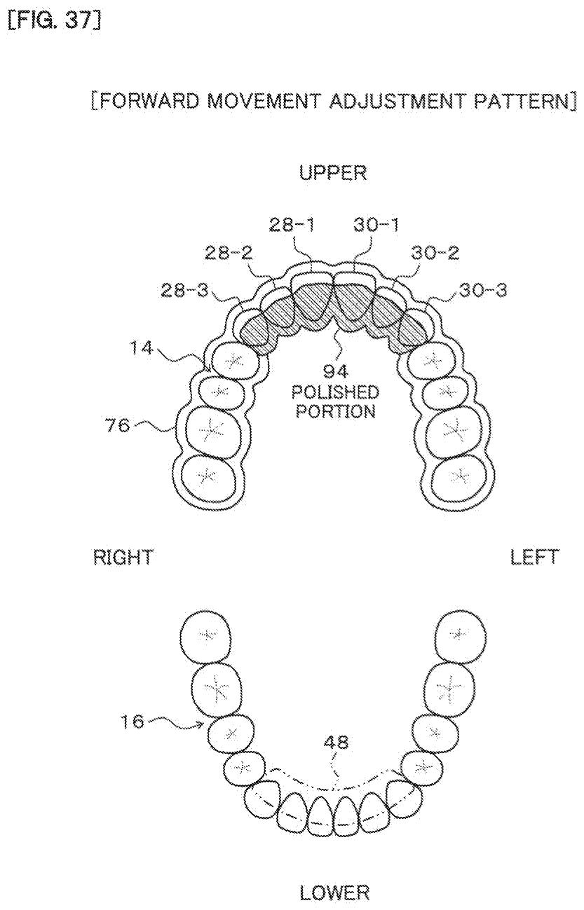

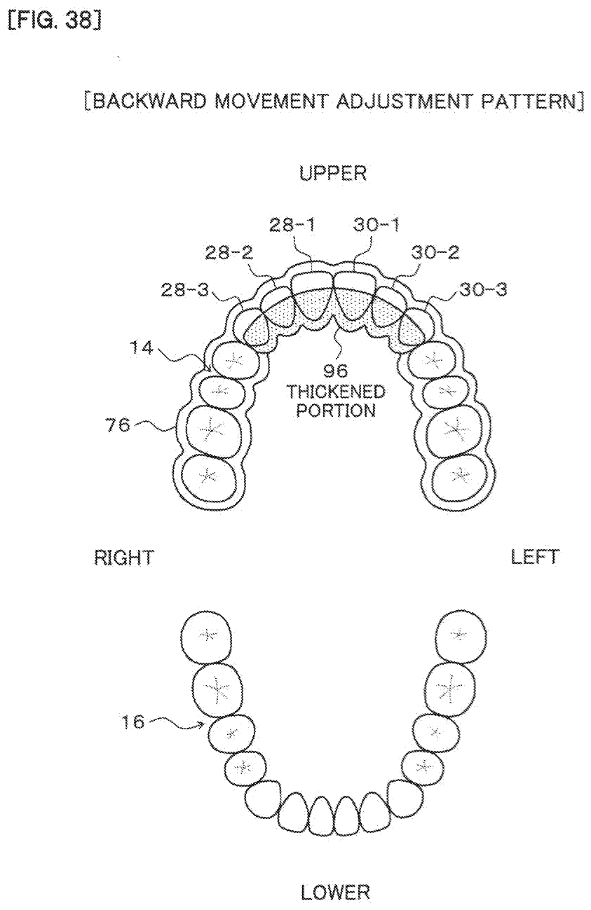

FIG. 36 is a flow diagram illustrating the details of the fine adjustment of the mouthpiece corresponding to the anteroposterior position of the mandibular position in step S54 of FIG. 24, FIG. 37 is an explanatory diagram illustrating a forward movement fine adjustment pattern in which a mouthpiece is finely adjusted when the head position in rest and relaxation is bent forward, and FIG. 38 is an explanatory diagram illustrating a backward movement fine adjustment pattern in which a mouthpiece is finely adjusted when a head position in rest and relaxation is bent backward.

As illustrated in FIG. 36, when the head position of the patient in rest and relaxation is a forward bending position in step S81, going to step S82 is performed, a polished portion 94 in which the thickness of the mouthpiece 76 located on the lingual tooth surface of the anterior teeth portion including the central incisors 28-1 and 30-1, the lateral incisors 28-2 and 30-2, and the cuspids 28-3 and 30-3 in the maxillary teeth dentition 14 is reduced by polishing in a unit of micron order is formed to make a forward movement adjustment pattern as illustrated in FIG. 37, and a tongue/jaw position reflex is induced to adjust the forward movement of the mandibular angle.

In contrast, when the head position of the patient in rest and relaxation is a backward bending position in step S83, going to step S84 is performed, a solvent of a vinyl material for producing a mouthpiece is applied to the lingual tooth surface of the anterior teeth portion including the central incisors 28-1 and 30-1, the lateral incisors 28-2 and 30-2, and the cuspids 28-3 and 30-3 in the maxillary teeth dentition 14, and is cured, thereby forming a thickened portion 96 of which the thickness is increased in a unit of micron order, to adjust the thickness and to form a backward movement adjustment pattern, and a tongue/jaw position reflex is induced to adjust the backward movement of the mandibular angle, as illustrated in FIG. 38.

The fine adjustment of the mouthpiece by the procedures of steps S51 to S54 in FIG. 24 described above allows a resting jaw position to be adequate so that the mandible is at a proper position. The chewing jaw position allowed to be adequate is achieved when the mouthpiece is produced in step S8 of FIG. 1. Therefore, when the fine adjustment of the mouthpiece is finished, the mouthpiece that enables both of the chewing jaw position and the resting jaw position to be appropriate is completed. When the patient mounts the completed mouthpiece on the maxillary teeth dentition, the lateral balance of the basal muscle tone of the posterior cervical region and the lateral balance of the basal muscle tone of the anterior cervical region becomes equivalent to each other at the time of both of chewing and resting, and the systemic basal muscle tone is balanced. As a result, a systemic pain symptom can be resolved.

[Hard-Type Mouthpiece]

When appropriate jaw movement can be confirmed in the soft-type mouthpiece produced by steps S1 to S9 of FIG. 1 to be also maintained in opening and closing mouth movement by allowing the chewing jaw position and the resting jaw position to be adequate, a mouthpiece is produced as a second-stage mouthpiece using a hard-type material so as to be in a form equivalent to that of the soft-type mouthpiece 76 produced in the procedures of steps S8 and S9 and finely adjusted in order to prevent the wear and degradation of a material.

[System for Supporting Production of Mouthpiece]

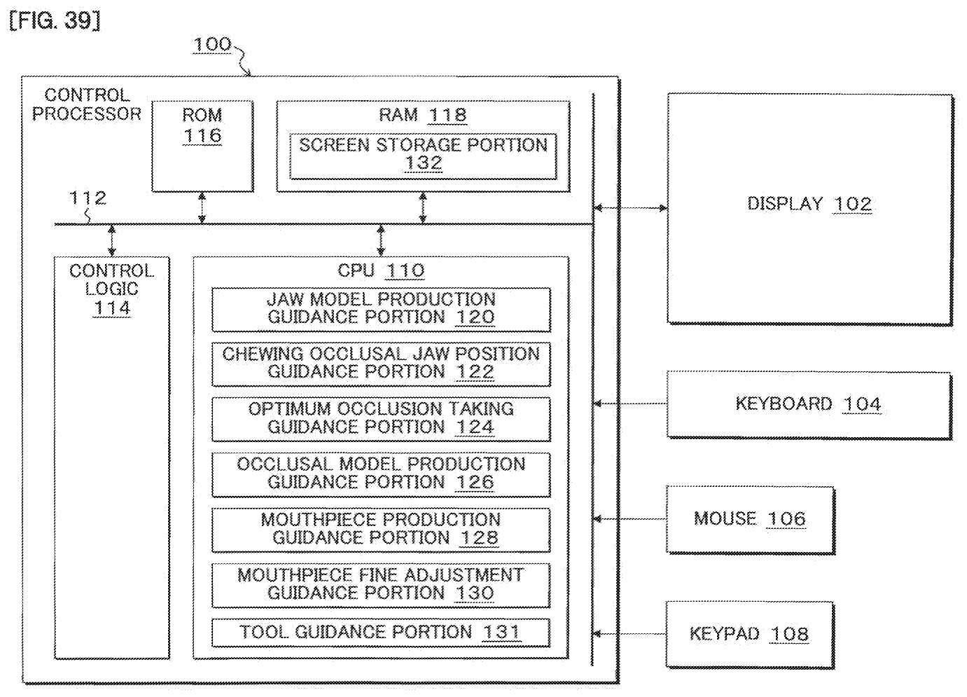

FIG. 39 is a block diagram illustrating an embodiment of a system for supporting production of a mouthpiece. As illustrated in FIG. 39, a system for supporting production of a mouthpiece of the present embodiment includes a control processor 100. A display 102 using a liquid crystal panel with a touch panel, or the like, a keyboard 104, a mouse 106, and a keypad 108 are disposed on the control processor 100.

A CPU 110 is disposed in the control processor 100, and a control logic 114, a ROM 116, and a RAM 118 are connected to a bus 112 from the CPU 110. The control logic 114 implements various hardware functions such as bus control associated with the control processing of the CPU 110.

The display 102, the keyboard 104, the mouse 106, and the keypad 108, disposed outside, are connected to the bus 112.

A jaw model production guidance portion 120 functioning as jaw model production guidance means, a chewing occlusal jaw position guidance portion 122 functioning as chewing occlusal jaw position guidance means, an optimum occlusion taking guidance portion 124 functioning as optimum occlusion taking guidance means, an occlusal model production guidance portion 126 functioning as occlusal model production guidance means, a mouthpiece production guidance portion 128 functioning as mouthpiece production guidance means, a mouthpiece fine adjustment guidance portion 130 functioning as mouthpiece fine adjustment guidance means, and a tool guidance portion 131 functioning as tool guidance means, as functions implemented by executing a program stored in the ROM 116, are disposed in the CPU 110.

A screen storage portion 132 is disposed in the RAM 118, and guidance screen information used for supporting production of a mouthpiece is stored in advance.

The system for supporting production of a mouthpiece illustrated in FIG. 39 is arranged integrally with or in the vicinity of, for example, a medical care table in a dental office, used by a doctor and a dentist as users, conducts, primarily through screen information on the display 102, guidance for supporting production of a mouthpiece that corrects the mandibular position at a proper position in order to optimize the balance of the systemic basal muscle tone of a patient complaining a systemic pain symptom caused by an unidentified complaint such as fibromyalgia, primarily according to diagnosis by the doctor and treatment operation by the dentist, and conducts audio guidance as needed.

[Scheme of Guidance Portion]

The jaw model production guidance portion 120 guides production of the maxillary model 10 and the mandibular model 12, which are illustrated in FIG. 3, and on which the teeth dentitions of the patient are arranged.

The chewing occlusal jaw position guidance portion 122 guides a medical care operation for constructing a chewing occlusal jaw position optimized for the patient.

The optimum occlusion taking guidance portion 124 guides production of the optimum occlusal impression member 70, which is illustrated in FIG. 21(B), and on which the occlusal teeth dentition of the optimized chewing occlusal jaw position is impressed, in a state in which a chewing occlusal jaw position optimized for the patient is constructed according to the guidance of the chewing occlusal jaw position guidance portion 122.

The occlusal model production guidance portion 126 guides an operation of fixing the optimum occlusal impression member 70 produced according to the guidance of the optimum occlusion taking guidance portion 124 to the occludator 52 in a state in which the optimum occlusal impression member 70 is sandwiched between the teeth dentitions of the maxillary model 10 and the mandibular model 12, to construct an optimized chewing occlusal model, as illustrated in FIG. 21(A).

The mouthpiece production guidance portion 128 guides production of the mouthpiece 76, which has an approximately uniform thickness, and on which the teeth dentition impression 82 of the maxillary model 10 and the teeth dentition impression 84 of the mandibular model 12 are formed, so that an optimized chewing occlusal jaw position is achieved, by the occludator 52 in which the chewing occlusal model is constructed, as illustrated in FIG. 22 and FIG. 23.

The mouthpiece fine adjustment guidance portion 130 guides a medical care operation of finely adjusting the mouthpiece 76 so that the systemic basal muscle tone of the patient is balanced to obtain an optimum resting jaw position in the resting jaw position state of the patient in which the mouthpiece 76 produced according to the guidance by the mouthpiece production guidance portion 128 is mounted.

The tool guidance portion 131 guides, as an option, for example, a method for using a tool such as a posture measurement instrument or an electromyograph which can be used in diagnosis for producing a mouthpiece.

Each guidance function will be described in detail as follows. The optimum occlusion taking guidance portion 124 and the occlusal model production guidance portion 126 have been already described, and therefore, detailed descriptions thereof are omitted.

[Chewing Occlusal Jaw Position Guidance Portion]

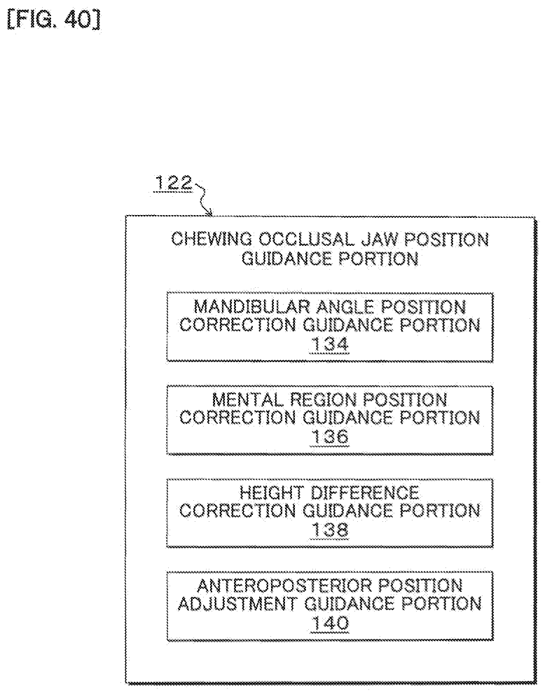

The chewing occlusal jaw position guidance portion 122 of FIG. 39 is provided with the functions of a mandibular angle position correction guidance portion 134 functioning as mandibular angle position correction guidance means, a mental region position correction guidance portion 136 functioning as mental region position correction guidance means, a height difference correction guidance portion 138 functioning as height difference correction guidance means, and an anteroposterior position adjustment guidance portion 140 functioning as anteroposterior position adjustment guidance means, as illustrated in FIG. 40.

(Mandibular Angle Position Correction Guidance Portion)

The mandibular angle position correction guidance portion 134 guides a medical care operation of correcting the lateral position of the mandibular angle at an optimized position using the tongue/jaw position reflex of the patient in correspondence with the enhancement of the basal muscle tone of the posterior cervical region of the patient, input through an operation of touching a screen displayed on the display 102 on the basis of the diagnosis results of the patient.

First, in the case of obtaining a diagnosis result in which no laterality occurs in the enhancement of the basal muscle tone of the posterior cervical region 20 of the patient as illustrated in FIG. 5(B), the mandibular angle position correction guidance portion 134 guides a medical care operation of preventing wax from being put on the lingual tooth surface of a mandibular molar tooth as illustrated in the first pattern of FIG. 5(A) on the basis of an operation of inputting the diagnosis result through the screen.

In the case of obtaining a diagnosis result in which the enhancement of the basal muscle tone of the right 20-1 of the posterior cervical region of the patient occurs as illustrated in FIG. 6(B), the mandibular angle position correction guidance portion 134 guides a medical care operation of putting the wax 32-1 on the lingual tooth surface of a mandibular right molar tooth to adjust the thickness of the wax 32-1 so that the laterality of the basal muscle tone of the posterior cervical region is eliminated, as illustrated in the second pattern of FIG. 6(B), on the basis of an operation of inputting the diagnosis result through the screen.

In the case of obtaining a diagnosis result in which the enhancement of the basal muscle tone of the left 20-2 of the posterior cervical region occurs as illustrated in FIG. 7(B), the mandibular angle position correction guidance portion 134 guides a medical care operation of putting the wax 32-2 on the lingual tooth surface of a mandibular left molar tooth to adjust the thickness of the wax so that the laterality of the basal muscle tone of the posterior cervical region is eliminated, as illustrated in the third pattern of FIG. 7(A), on the basis of the input of the diagnosis result.

(Mental Region Position Correction Guidance Portion)

The mental region position correction guidance portion 136 guides a medical care operation of correcting the lateral position of the mental region at an optimized position using the tongue/jaw position reflex of the patient in correspondence with the enhancement of the basal muscle tone of the anterior cervical region of the patient, input through an operation of touching a screen displayed on the display 102 on the basis of the diagnosis results of the patient.

First, in the case of obtaining a diagnosis result in which no laterality occurs in the enhancement of the basal muscle tone of the anterior cervical region 22 of the patient as illustrated in FIG. 5(B), the mental region position correction guidance portion 136 guides a medical care operation of preventing wax from being put on the lingual tooth surface of a mandibular premolar tooth as illustrated in the first pattern of FIG. 5(A) on the basis of an operation of inputting the diagnosis result through the screen.

In the case of obtaining a diagnosis result in which the enhancement of the basal muscle tone of the right 22-1 of the anterior cervical region of the patient occurs as illustrated in FIG. 9(B), the mental region position correction guidance portion 136 guides a medical care operation of putting the wax 34-1 on the lingual tooth surface of a mandibular right premolar tooth to adjust the thickness of the wax 34-1 so that the laterality of the basal muscle tone of the anterior cervical region is eliminated, as illustrated in the fourth pattern of FIG. 9(A), on the basis of an operation of inputting the diagnosis result through the screen, when the result is input.

In the case of obtaining a diagnosis result in which the enhancement of the basal muscle tone of the left 22-2 of the anterior cervical region of the patient occurs as illustrated in FIG. 10(B), the mental region position correction guidance portion 136 guides a medical care operation of putting the wax 34-2 on the lingual tooth surface of a mandibular left premolar tooth to adjust the thickness of the wax 34-2 so that the laterality of the basal muscle tone of the anterior cervical region is eliminated, as illustrated in the fifth pattern of FIG. 10(A), on the basis of an operation of inputting the diagnosis result through the screen.

Further, a treatment operation of putting wax on each of the lingual tooth surfaces of molar and premolar teeth in any of the sixth to ninth patterns illustrated in FIG. 11 to FIG. 14 to balance the laterality of the basal muscle tone of the posterior or anterior cervical region is guided through the screen information of the display 102 on the basis of an operation of inputting, through the screen, a diagnosis result as a combination of the enhancement of the basal muscle tones of the posterior cervical region and the anterior cervical region by the mandibular angle position correction guidance portion 134 and the mental region position correction guidance portion 136.

(Height Difference Correction Guidance Portion)

The height difference correction guidance portion 138 guides, through the screen information of the display 102, a medical care operation of correcting a difference between the heights of the mandibular angle and the mental region using the masseter/jaw position reflex of the patient in a state in which corrections according to the guidance of the mandibular angle position correction guidance portion 134 and the guidance of the mental region position correction guidance portion 136 have been finished.

In other words, the height difference correction guidance portion 138 guides a medical care operation of putting wax on the occlusal surfaces of both maxillary first molar teeth to configure a chewing jaw position, measuring a forced expiratory residual volume with a spirometer after induction of a masseter/jaw position reflex, and correcting the elevation angle of the occlusal reference plane of a mandibular teeth dentition with respect to a horizontal reference plane so that the forced expiratory residual volume is not more than a predetermined value.

In this case, the height difference correction guidance portion 138 guides a medical care operation of correcting the elevation angle so that the forced expiratory residual volume is 500 milliliters or less.

The height difference correction guidance portion 138 guides a medical care operation of preparing the mini-splint 46 in which the predetermined, corrected elevation angle θ of the occlusal reference plane 42 of the mandibular teeth dentition with respect to the horizontal reference plane 40 is set, and mounting the mini-splint 46 on the mandibular teeth dentition to measure the forced expiratory residual volume with the spirometer, as illustrated in FIG. 16, when the forced expiratory residual volume is not 500 milliliters or less.

The height difference correction guidance portion 138 guides a medical care operation of preparing a plurality of kinds of mini-splints 46 of which the elevation angle-corrected angles θ are different, selecting a mini-splint 46 having a predetermined elevation angle-corrected angle from the mini-splints, and using the mini-splint 46, as illustrated in FIG. 17.

(Anteroposterior Position Adjustment Guidance Portion)

The anteroposterior position adjustment guidance portion 140 guides a medical care operation of finely adjusting the anteroposterior position of a chewing occlusal jaw position so that the head position of the patient in rest and relaxation is maintained at a midline position with respect to the visual line of the patient.

In other words, the anteroposterior position adjustment guidance portion 140 guides a medical care operation of moving forward a mandibular position when the head position of the patient in the rest and relaxation is a forward bending position and moving backward the mandibular position when the head position is a backward bending position.

Specifically, in a case in which the patient is incapable of single-handedly finely adjusting an anteroposterior position, the anteroposterior position adjustment guidance portion 140 guides a medical care operation of putting the wax 48 on the lingual tooth surface of the mandibular anterior teeth portion to adjust the thickness of the wax so that forward movement is assisted when the head position of the patient in the rest and relaxation is a forward bending position, as illustrated in FIG. 19, and guides a medical care operation of putting the wax 50 on the lingual tooth surface of the maxillary anterior teeth portion to adjust the thickness of the wax 50 so that backward movement is assisted when the head position of the patient in the rest and relaxation is a backward bending position, as illustrated in FIG. 20.

[Mouthpiece Production Guidance Portion]

The mouthpiece production guidance portion 128 in FIG. 39 first guides production of a mouthpiece part on which the maxillary teeth dentition is carved based on the maxillary model 10, and which has an approximately uniform thickness.

Then, the mouthpiece production guidance portion 128 guides an operation of sandwiching the optimum occlusal impression member 70 between the maxillary model 10 and the mandibular model 12 to fix the maxillary model 10 and the mandibular model to the occludator 52 to reproduce the optimum chewing occlusal jaw position, as illustrated in FIG. 21.

Then, the mouthpiece production guidance portion 128 guides an operation of mounting a mouthpiece part 76a on the maxillary model 10 of the occludator 52 from which the optimum occlusal impression member 70 is removed, and applying the teeth dentition impression 84 of the mandibular model to an occlusal surface of the mouthpiece part 76a on the basis of occlusion with the mandibular model 12 via the mouthpiece part 76a to complete the mouthpiece 76 illustrated in FIG. 23, as illustrated in FIG. 22.

[Mouthpiece Fine Adjustment Guidance Portion]

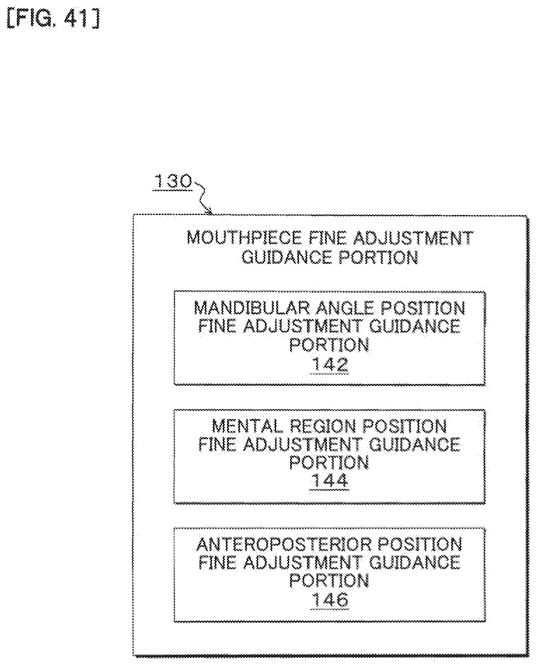

The mouthpiece fine adjustment guidance portion 130 is provided with the functions of a mandibular angle position fine adjustment guidance portion 142 functioning as mandibular angle position fine adjustment guidance means, a mental region position fine adjustment guidance portion 144 functioning as mental region position fine adjustment guidance means, and an anteroposterior position fine adjustment guidance portion 146 functioning as anteroposterior position fine adjustment guidance means, as illustrated in FIG. 41.

(Mandibular Angle Position Fine Adjustment Guidance Portion)

In the case of obtaining a diagnosis result in which there is no enhancement of the basal muscle tone of the posterior cervical region 20 of the patient, as illustrated in FIG. 26(B), in the resting jaw position state of the patient in which the completed mouthpiece is mounted on the maxillary teeth dentition of the patient, the mandibular angle position fine adjustment guidance portion 142 guides a fine adjustment operation in which fine adjustment of the mouthpiece 76 by polishing is not performed, as illustrated in the first adjustment pattern in FIG. 26(A), on the basis of an operation of inputting the diagnosis result through the screen.

In the case of obtaining a diagnosis result in which the enhancement of the basal muscle tone of the right 20-1 of the posterior cervical region of the patient occurs as illustrated in FIG. 27(B), the mandibular angle position fine adjustment guidance portion 142 guides a fine adjustment operation of polishing the thickness of the mouthpiece 76 located in the lingual tooth surface of a maxillary right molar tooth to reduce the thickness of the mouthpiece 76 so that the laterality of the basal muscle tone of the posterior cervical region is eliminated, as illustrated in the polished portion 90-1 of the second adjustment pattern in FIG. 27(A), on the basis of an operation of inputting the diagnosis result through the screen.

In the case of obtaining a diagnosis result in which the enhancement of the basal muscle tone of the left 20-2 of the posterior cervical region of the patient occurs as illustrated in FIG. 28(B), the mandibular angle position fine adjustment guidance portion 142 guides a fine adjustment operation of polishing the thickness of the mouthpiece 76 located in the lingual tooth surface of a maxillary left molar tooth to reduce the thickness of the mouthpiece 76 so that the laterality of the basal muscle tone of the posterior cervical region is eliminated, as illustrated in the polished portion 90-2 in FIG. 28(A), on the basis of an operation of inputting the diagnosis result through the screen.

(Mental Region Position Fine Adjustment Guidance Portion)

In the case of obtaining a diagnosis result in which there is no laterality of the basal muscle tone of the anterior cervical region 22 of the patient, as illustrated in FIG. 26(B), in the resting jaw position state of the patient in which the completed mouthpiece is mounted on the maxillary teeth dentition of the patient, the mental region position fine adjustment guidance portion 144 guides a fine adjustment operation in which fine adjustment of the mouthpiece 76 by polishing is not performed, as illustrated in the first adjustment pattern in FIG. 26(A), on the basis of an operation of inputting the diagnosis result through the screen.

In the case of obtaining a diagnosis result in which the enhancement of the basal muscle tone of the right 22-1 of the anterior cervical region of the patient occurs as illustrated in FIG. 30(B), the mental region position fine adjustment guidance portion 144 guides a fine adjustment operation of polishing the thickness of the mouthpiece 76 located in the lingual tooth surface of a maxillary right premolar tooth to reduce the thickness of the mouthpiece 76 so that the laterality of the basal muscle tone of the anterior cervical region is eliminated, as illustrated in the polished portion 92-1 in FIG. 30(A), on the basis of an operation of inputting the diagnosis result through the screen.

In the case of obtaining a diagnosis result in which the enhancement of the basal muscle tone of the left 22-2 of the anterior cervical region occurs as illustrated in FIG. 31(B), the mental region position fine adjustment guidance portion 144 guides a fine adjustment operation of polishing the thickness of the mouthpiece 76 located in the lingual tooth surface of a maxillary left premolar tooth to reduce the thickness of the mouthpiece 76 so that the laterality of the basal muscle tone of the anterior cervical region is eliminated, as illustrated in the polished portion 92-2 in FIG. 31(A), on the basis of an operation of inputting the diagnosis result through the screen.

Further, a treatment operation of polishing each of the lingual tooth surfaces of molar and premolar teeth inanyof the sixth to ninth adjustment patterns illustrated in FIG. 32 to FIG. 35 to reduce the thickness thereof, thereby balancing the laterality of the basal muscle tone of the posterior or anterior cervical region is guided through the screen information of the display 102 on the basis of an operation of inputting, through the screen, a diagnosis result in a combination of the enhancement of the basal muscle tones of the posterior cervical region and the anterior cervical region by the mandibular angle position fine adjustment guidance portion 142 and the mental region position fine adjustment guidance portion 144.

(Anteroposterior Position Fine Adjustment Guidance Portion)

In the case of obtaining a diagnosis result in which the head position of the patient in rest and relaxation is a forward bending position in the resting jaw position state of the patient in which the completed mouthpiece is mounted on the maxillary teeth dentition of the patient, the anteroposterior position fine adjustment guidance portion 146 guides a fine adjustment operation of polishing the thickness of the mouthpiece 76 located on the lingual tooth surface of the maxillary anterior teeth portion to reduce the thickness of the mouthpiece 86 so that forward movement of the mandible is assisted, as illustrated in the polished portion 94 in FIG. 37, on the basis of inputting the diagnosis result through the screen.

In obtainment of a diagnosis result in which the head position of the patient in rest and relaxation is a backward bending position in the resting jaw position state of the patient in which the completed mouthpiece is mounted on the maxillary teeth dentition of the patient, the anteroposterior position fine adjustment guidance portion 146 guides a fine adjustment operation of thickening the thickness of the mouthpiece 76 located on the lingual tooth surface of the maxillary anterior teeth portion to increase the thickness of the mouthpiece 76 so that backward movement of the mandible is assisted, as illustrated in the thickened portion 96 in FIG. 38, on the basis of inputting the diagnosis result through the screen.

[Guidance Screen]

An example of a guidance screen displayed on the display 102 by the system for supporting production of a mouthpiece in FIG. 39 will be described as follows.

(Guidance Screen of Main Menu)

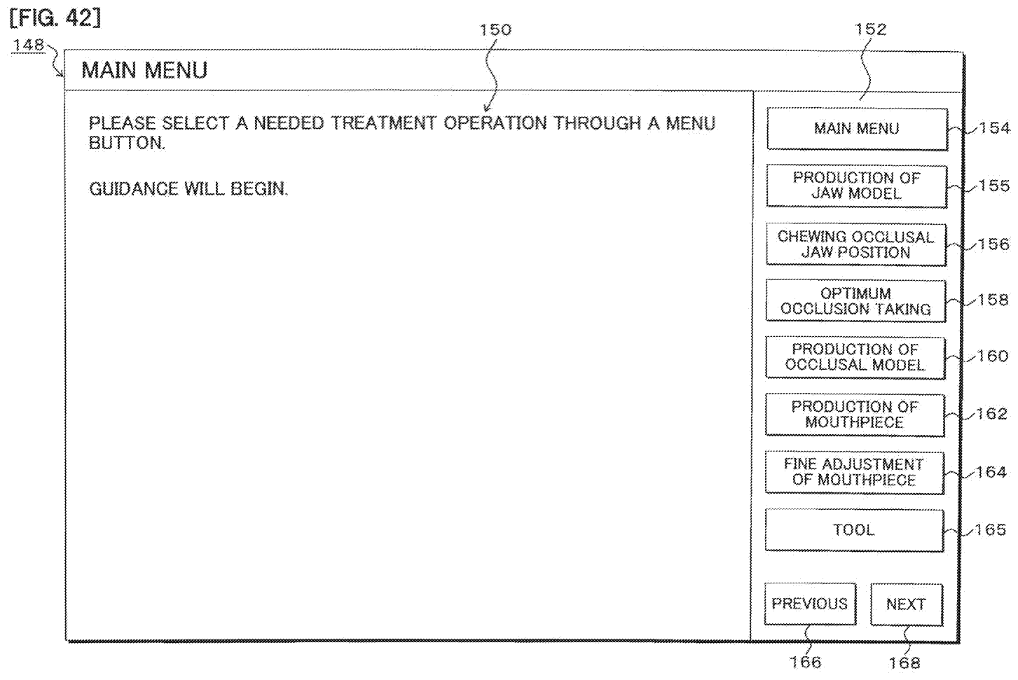

FIG. 42 is an explanatory diagram illustrating a guidance screen of a main menu. As illustrated in FIG. 42, a guidance screen 148 of a main menu is provided with a guidance 150 indicating the start of a treatment operation and a menu selector 152.

The menu selector 152 is provided with selection buttons 154, 155, 156, 158, 160, 162, 164, and 165 corresponding to operation items of a main menu, production of a jaw model, a chewing occlusal jaw position, optimum occlusion taking, production of an occlusal model, production of a mouthpiece, fine adjustment of a mouthpiece, and a tool. Moreover, the menu selector 152 is provided with a “previous” button 166 and a “next” button 168 used for switching a screen.

A user of the system in turn selecting down the selection buttons 154, 156, 158, 160, 162, and 164 of the menu selector 152 through touch operation, whereby guidance for producing a mouthpiece is performed using a guidance screen.

(Screen for Guidance of Production of Jaw Model)

An operation of touching the selection button 154 in FIG. 42 results in switching to a screen for guidance of production of a model die. The screen for guidance of production of a model die indicates a title of “Production of Jaw Model” in the upper portion of the screen in FIG. 42 and guidance that “The maxillary and mandibular models of a patient are produced using a bite material.” in a portion of the guidance 150, which are not illustrated.

[Screen for Guidance of Chewing Occlusal Jaw Position]

(Guidance Screen for Correcting Mandibular Angle and Mental Region)

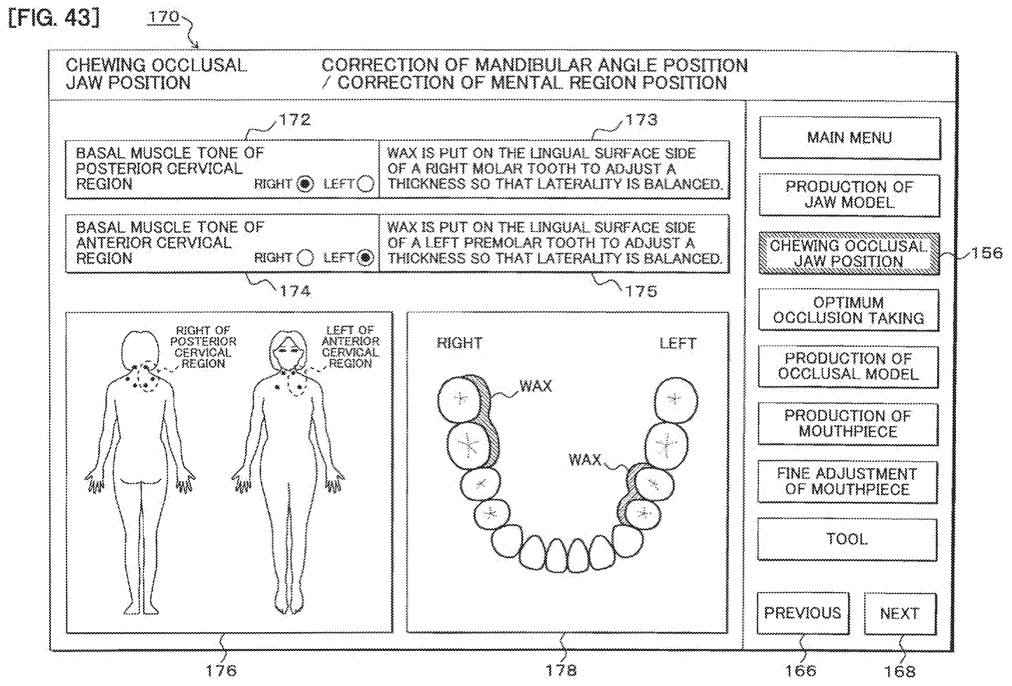

FIG. 43 is an explanatory diagram illustrating a guidance screen for correcting the mandibular angle and the mental region. The screen is switched and displayed by selecting “Chewing Occlusal Jaw Position” by the operation of the selection button 156 in FIG. 42.

As illustrated in FIG. 43, a guidance screen 170 for correcting the mandibular angle and the mental region displays input portions 172 and 174, a diagnosis image 176, and a medical care operation image 178, illustrated by taking, as an example, the diagnosis result illustrated in FIG. 11(B).

In the input portion 172, the diagnosis result of the basal muscle tone of the posterior cervical region of the patient is input through a right radio button or a left radio button. In the diagnosis result in FIG. 11(B), the right radio button is operated because the enhancement of the basal muscle tone occurs in the right of the posterior cervical region. In correspondence with such an operation, a treatment operation guidance 173 is displayed on the right.

In the input portion 174, the diagnosis result of the basal muscle tone of the anterior cervical region of the patient is input through a right radio button or a left radio button. In the diagnosis result in FIG. 11(B), the left radio button is operated because the enhancement of the basal muscle tone occurs in the left of the anterior cervical region. In correspondence with such an operation, a treatment operation guidance 175 is displayed on the right.

The diagnosis image 176 is the same image as the illustration in FIG. 11(B), and the medical care operation image 178 is the same image as the mandibular teeth dentition illustrated in FIG. 11(A).

In addition, a guidance screen corresponding to the diagnosis result and the medical care operation illustrated in any of FIGS. 5 to 7 and FIGS. 10 to 14 is displayed on the guidance screen 170 for correcting the mandibular angle and the mental region by an operation of inputting a diagnosis result through the input portions 172 and 174.

(Guidance Screen for Correcting Difference between Heights)

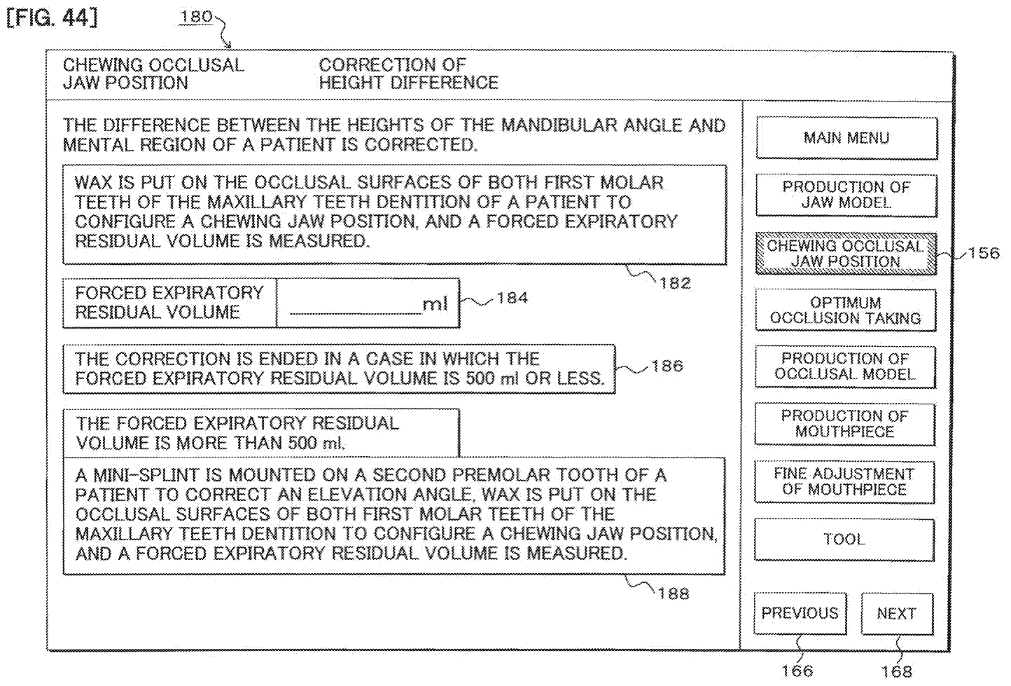

FIG. 44 is an explanatory diagram illustrating a guidance screen for correcting a difference between the heights of the mandibular angle and the mental region. The screen is switched by operating the “next” button 168 of the guidance screen 170 in FIG. 43.

As illustrated in FIG. 44, guidances 182, 184, 186, and 188 are displayed on a guidance screen 180 for correcting a difference between the heights of the mandibular angle and the mental region. The guidance 182 indicates an operation of measuring a forced expiratory residual volume. The guidance 184 indicates the input frame of the measurement value of the forced expiratory residual volume. The guidance 186 indicates that the correction is ended in a case in which the forced expiratory residual volume is 500 mL or less. The guidance 188 indicates a medical care operation using a mini-splint in a case in which the forced expiratory residual volume is more than 500 mL.

(Guidance Screen for Assisting Forward Movement in Adjustment of Anteroposterior Position)

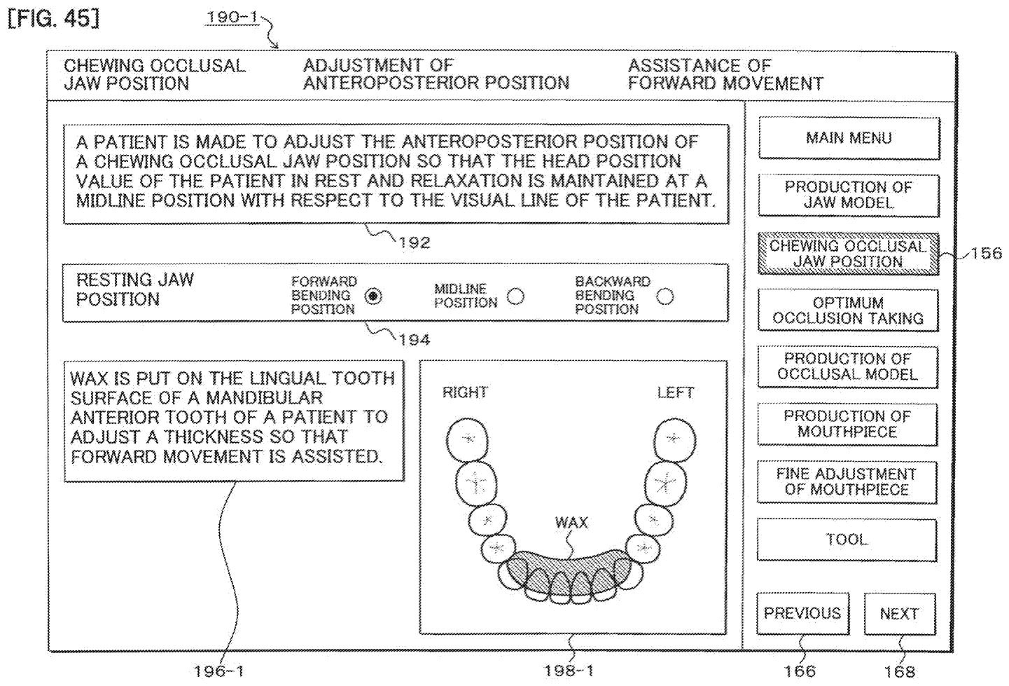

FIG. 45 is an explanatory diagram illustrating a guidance screen for assisting the forward movement of the chewing jaw position. The screen is switched by operating the “next” button 168 in FIG. 44.

As illustrated in FIG. 45, a guidance screen 190-1 for assisting forward movement is provided with a guidance 192, an input portion 194, a guidance 196-1, and a medical care operation screen 198-1.

The guidance 192 indicates a medical care operation for diagnosing the anteroposterior posture of the patient. In the input portion 194, which is intended to input the diagnosis result of the resting jaw position, radio buttons corresponding to a forward bending position, a midline position, and a backward bending position are arranged.

When the diagnosis result in the rest is a forward bending position, the radio button for the forward bending position is operated. In correspondence with such an operation, the guidance 196-1 indicates a medical care operation for assisting the forward movement of the patient and simultaneously indicates the medical care operation image 198-1. The medical care operation image 198-1 of the present embodiment is the same image as that of the mandibular teeth dentition 16 in the forward movement pattern illustrated in FIG. 19.

(Guidance Screen for Assisting Backward Movement in Adjustment of Anteroposterior Position)

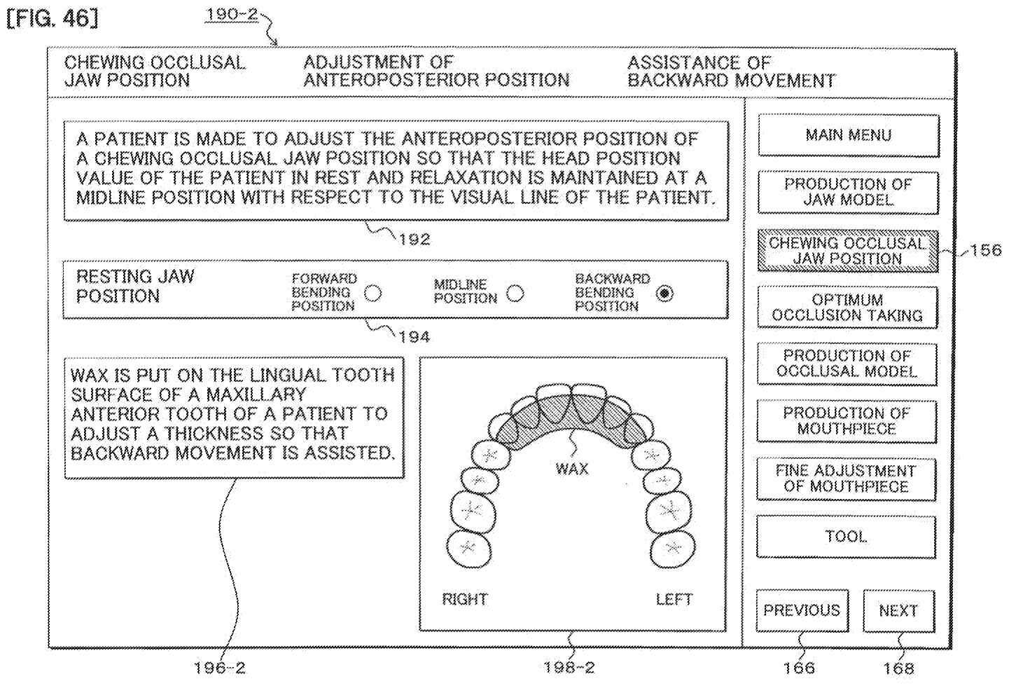

FIG. 46 is an explanatory diagram illustrating a guidance screen for assisting the backwardmovement of the chewing jaw position. The screen is switched by operating the “next” button 168 in FIG. 45.

As illustrated in FIG. 46, a guidance screen 190-2 for assisting backward movement is provided with a guidance 192, an input portion 194, a guidance 196-2, and a medical care operation screen 198-2.

The guidance 192 and the input portion 194 are the same as those in FIG. 45. When a diagnosis result in rest is a backward bending position, the radio button for the backward bending position is operated. In correspondence with such an operation, the guidance 196-2 indicates a medical care operation for assisting the backward movement of the patient and simultaneously indicates the medical care operation image 198-2. The medical care operation image 198-2 of the present embodiment is the same image as that of the maxillary teeth dentition 46 in the forward movement pattern illustrated in FIG. 20.

[Guidance Screen for Taking Optimum Occlusion]

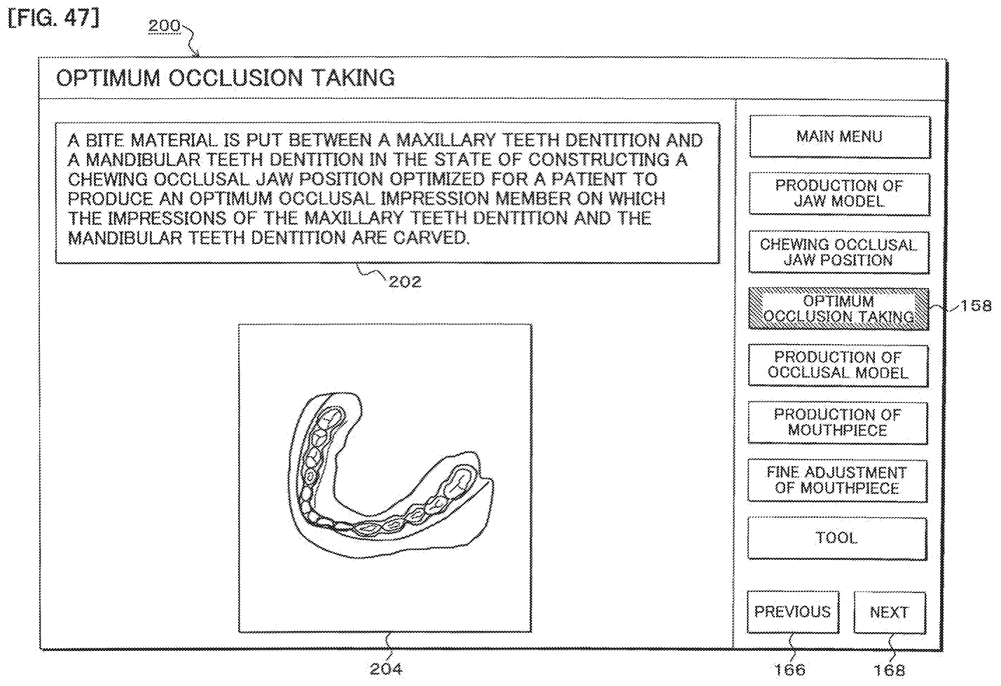

FIG. 47 is an explanatory diagram illustrating a guidance screen for taking optimum occlusion. The screen is switched in the case of operating the selection button 158 in FIG. 42 or operating the “next” button 168 in FIG. 46.

As illustrated in FIG. 47, a guidance screen 200 for taking optimum occlusion displays a guidance 202 and an image 204. The guidance 202 indicates an operation of producing an optimum occlusal impression member is using a bite material. The image 204 indicates the produced optimum occlusal impression member which is the same as that illustrated in FIG. 21(B).

[Guidance Screen for Producing Occlusal Model]

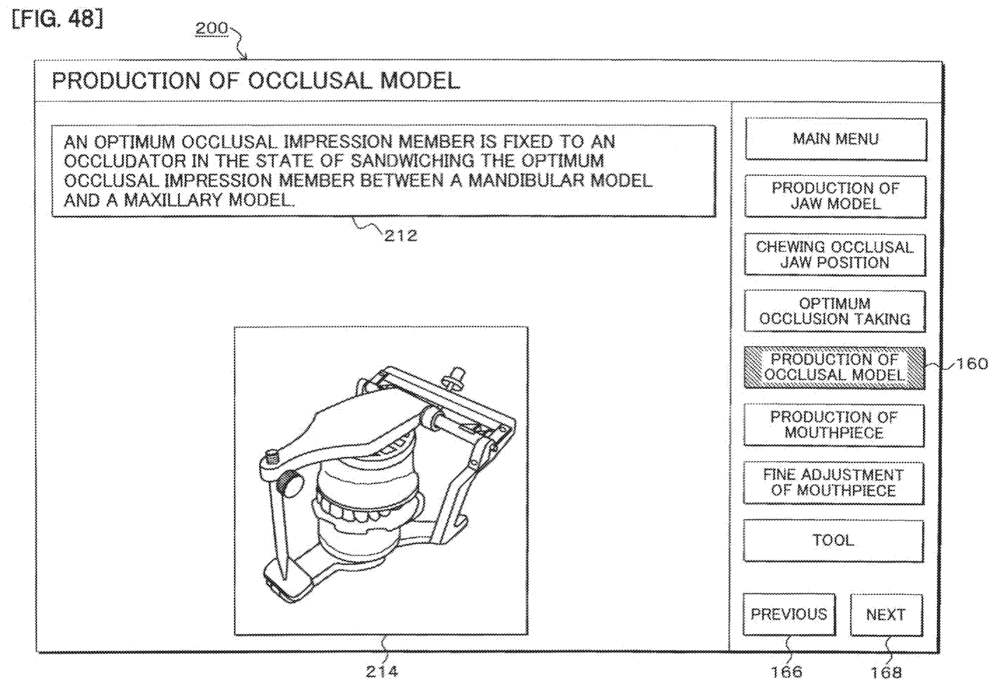

FIG. 48 is an explanatory diagram illustrating a guidance screen for producing an occlusal model. The screen is switched in the case of operating the selection button 160 in FIG. 42 or operating the “next” button 168 in FIG. 47.

As illustrated in FIG. 48, a guidance screen 210 for producing an occlusal model displays a guidance 212 and an image 214. The guidance 212 indicates an operation of constructing an occlusal model by an occludator using an optimum occlusal impression member. The image 214 indicates an image of the same occludator in which an occlusal model is constructed as that illustrated in FIG. 21(A).

[Guidance Screen for Producing Mouthpiece]

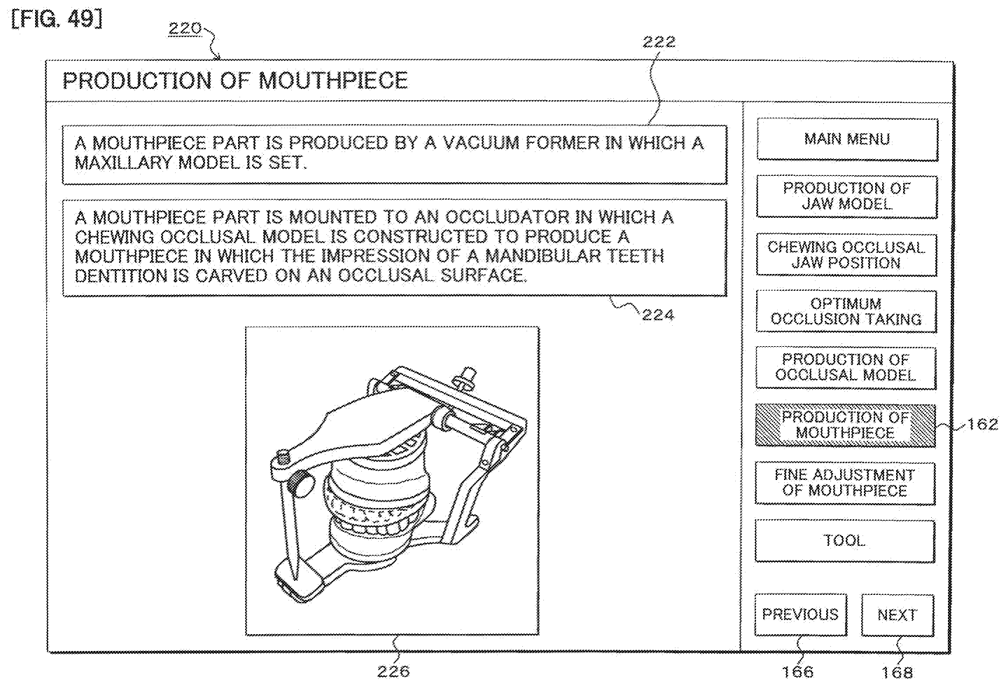

FIG. 49 is an explanatory diagram illustrating a guidance screen for producing a mouthpiece. The screen is switched in the case of operating the selection button 162 in FIG. 42 or operating the “next” button 168 in FIG. 47.

As illustrated in FIG. 49, a guidance screen 220 for producing a mouthpiece displays guidances 222 and 224 and an image 226. The guidance 222 indicates an operation of producing a mouthpiece part using a vacuum former.

The guidance 224 indicates an operation of carving the impression of the tongue jaw teeth dentition on the occlusal surface of the mouthpiece part using an occludator. Further, the image 226 indicates an image in which a mouthpiece is rapidly completed using the same occludator as the occludator illustrated in FIG. 22. In addition to a still image, a moving image in which the mouthpiece is produced using the occludator may be replayed and displayed as the image 226.

[Guidance Image for Fine Adjustment of Mouthpiece]

(Guidance Screen for Fine Adjustment of Mouthpiece Corresponding to Mandibular Angle and Mental Region)

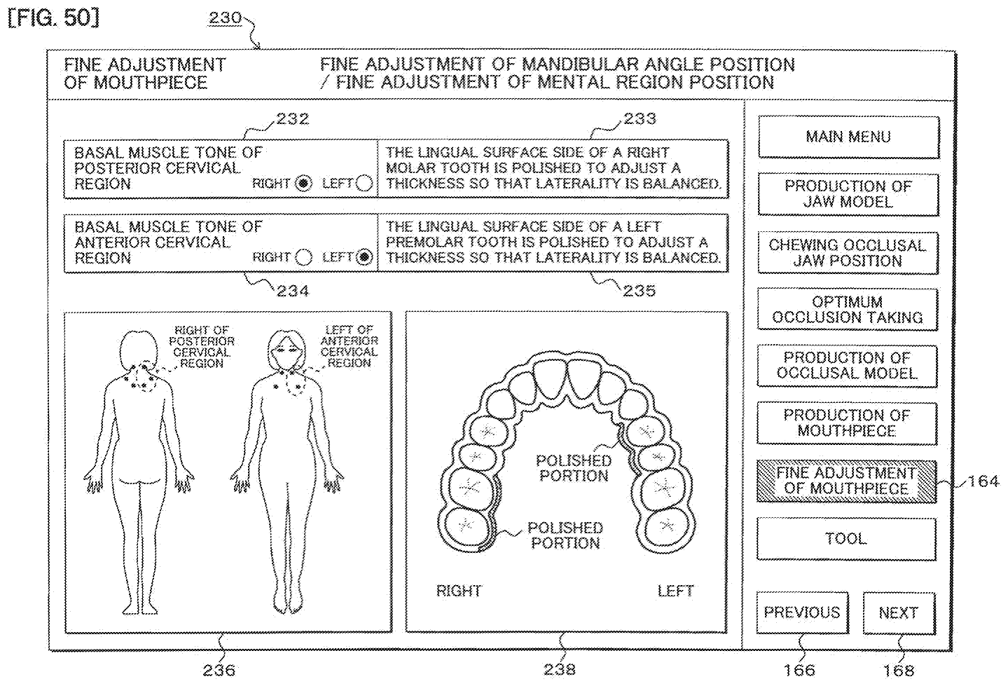

FIG. 50 is an explanatory diagram illustrating a guidance screen for performing fine adjustment of a mouthpiece, corresponding to the position of the mandibular angle and the position of the mental region. The screen is switched and displayed in the case of operating the selection button 164 in FIG. 42 or operating the “next” button 168 in FIG. 49.

As illustrated in FIG. 50, a guidance screen 230 for fine adjustment of a mouthpiece corresponding to the mandibular angle and the mental region displays input portions 232 and 234, treatment operation guidances 233 and 235, a diagnosis image 236, and a medical care operation image 238, illustrated by taking, as an example, the diagnosis result illustrated in FIG. 32(B).

In the input portion 232, the diagnosis result of the basal muscle tone of the posterior cervical region of the patient is input through a right radio button or a left radio button. In the diagnosis result in FIG. 32(B), the right radio button is operated because the enhancement of the basal muscle tone occurs in the right of the posterior cervical region. In correspondence with such an operation, the treatment operation guidance 233 is displayed on the right.

In the input portion 234, the diagnosis result of the basal muscle tone of the anterior cervical region of the patient is input through a right radio button or a left radio button. In the diagnosis result in FIG. 32(B), the left radio button is operated because the enhancement of the basal muscle tone occurs in the left of the anterior cervical region. In correspondence with such an operation, the treatment operation guidance 235 is displayed on the right.

The diagnosis image 236 is the same image as the illustration in FIG. 32(B), and the medical care operation image 238 is the same image as the illustration of the polished portions 90-1 and 92-2 of the maxillary teeth dentition 14 on which the mouthpiece 76 is mounted, illustrated in FIG. 32(A).

In addition, a screen corresponding to the diagnosis result and the medical care operation illustrated in any of FIGS. 26 to 28 and FIGS. 31 to 35 is displayed on the guidance screen 230 for fine adjustment of a mouthpiece corresponding to the mandibular angle and the mental region by inputting a diagnosis result through the input portions 232 and 234.

(Guidance Screen for Assisting Forward Movement in Adjustment of Anteroposterior Position)

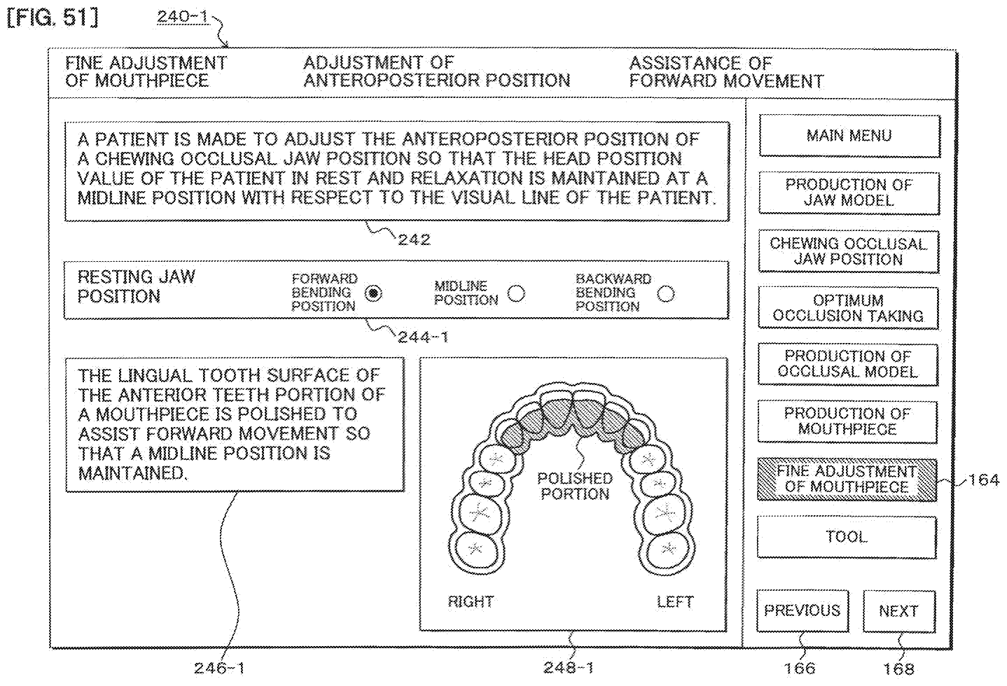

FIG. 51 is an explanatory diagram illustrating a guidance screen for performing fine adjustment of a mouthpiece, corresponding to the assistance of the forward movement of the chewing jaw position. The screen is switched and displayed by operating the “next” button 168 in FIG. 50.

As illustrated in FIG. 51, a guidance screen 240-1 for fine adjustment of a mouthpiece corresponding to assistance of forward movement displays a guidance 242, an input portion 244-1, a guidance 246-1, and a medical care operation image 248-1.

The guidance 242 indicates a medical care operation for diagnosing the anteroposterior posture of the patient. In the input portion 244-1, which is intended to input the diagnosis result of the resting jaw position, radio buttons corresponding to a forward bending position, a midline position, and a backward bending position are arranged.

When the diagnosis result in the rest is a forward bending position, the radio button for the forward bending position in the guidance 244-1 is operated. In correspondence with such an operation, the guidance 246-1 indicates a medical care operation for assisting the forward movement of the patient and simultaneously indicates the medical care operation image 248-1. The medical care operation image 248-1 of the present embodiment is the same image as the image illustrating the polished portion 94 of the mouthpiece 76 mounted on the maxillary teeth dentition 14 in the forward movement pattern illustrated in FIG. 37.

(Guidance Screen for Assisting Backward Movement in Adjustment of Anteroposterior Position)

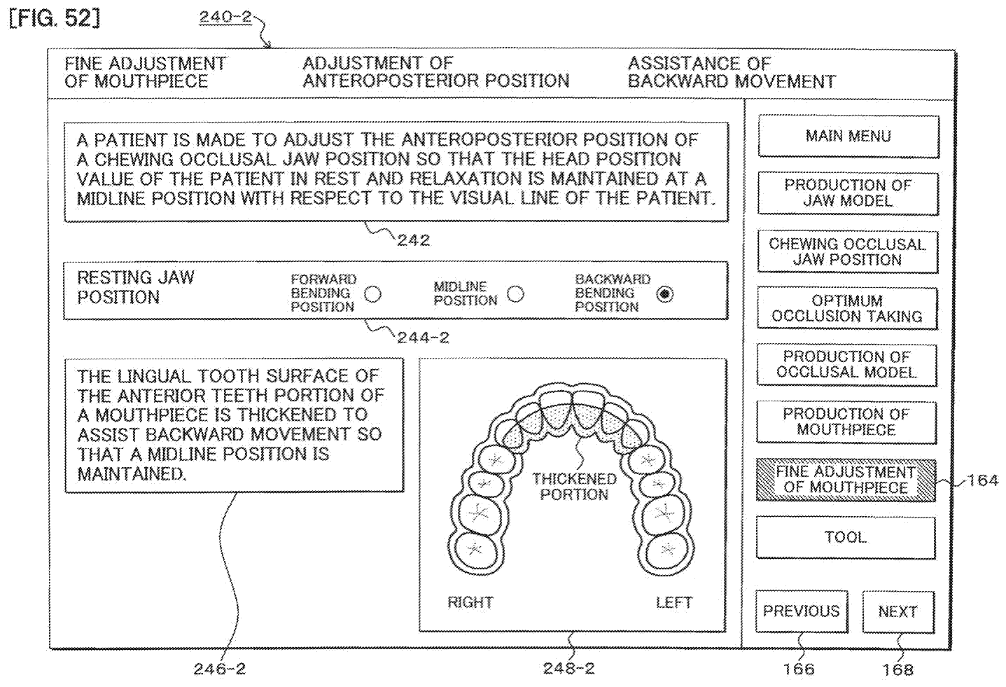

FIG. 52 is an explanatory diagram illustrating a guidance screen for performing fine adjustment of a mouthpiece, corresponding to the assistance of the backward movement of the chewing jaw position . The screen is switched and displayed by operating the “next” button 168 in FIG. 50.

As illustrated in FIG. 52, a guidance screen 240-2 for assisting backward movement displays a guidance 242, an input portion 244-2, a guidance 246-2, and a medical care operation image 248-2.

The guidance 242 and the input portion 244-2 are the same as those in FIG. 51. When the diagnosis result in the rest is a backward bending position, the radio button for the backward bending position in the input portion 244-2 is operated. In correspondence with such an operation, the guidance 246-2 indicates a medical care operation for assisting the backward movement of the patient and simultaneously indicates the medical care operation image 248-2. The medical care operation image 248-2 of the present embodiment is the same image as the image illustrating the thickened portion 96 of the mouthpiece 76 mounted on the maxillary teeth dentition 14 in the backward movement pattern illustrated in FIG. 38.

In the embodiment in FIG. 39, whether or not the enhancement of the basal muscle tone occurs in the posterior and anterior cervical regions of the patient is diagnosed by a user such as a dentist. However, whether or not the enhancement of the basal muscle tone occurs may be automatically detected by using an appropriate sensor that detects whether or not the enhancement of the basal muscle tone occurs, and may be input into the control processor 100.

[Guidance Screen for Tool]

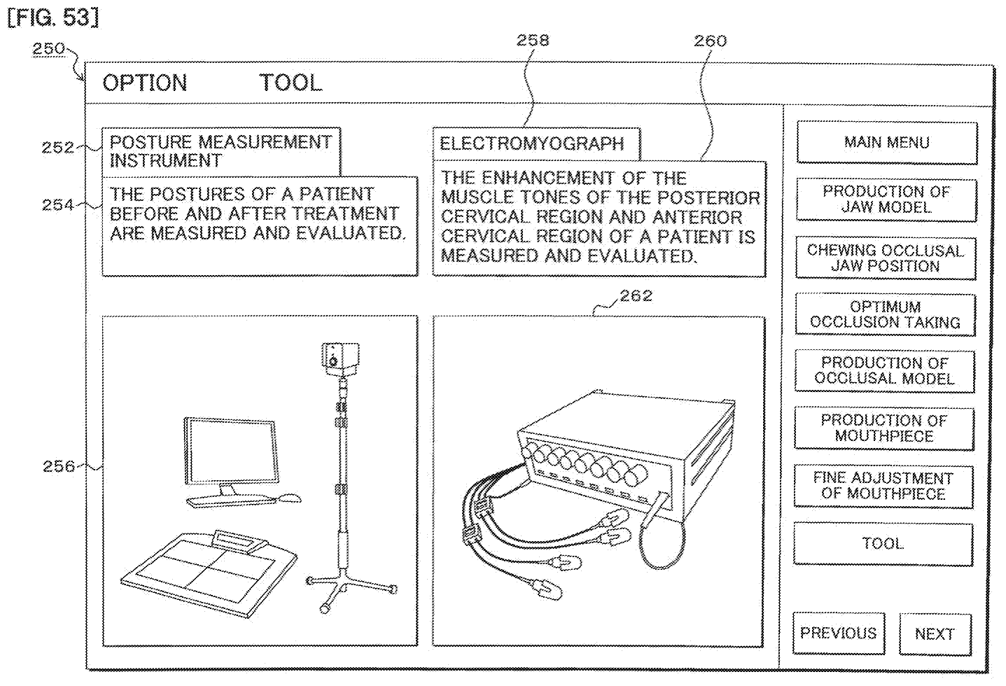

FIG. 53 is an explanatory diagram illustrating a guidance screen displayed by a tool guidance portion. The screen is displayed by touching and operating the selection button 165 indicating a tool in the menu selector 152.

As illustrated in FIG. 53, a posture measurement instrument selection button 252, a posture measurement instrument guidance 254, a posture measurement device image 256, an electromyograph selection button 258, an electromyograph guidance 260, and an electromyograph image 262 are displayed on a guidance screen 250 for a tool.

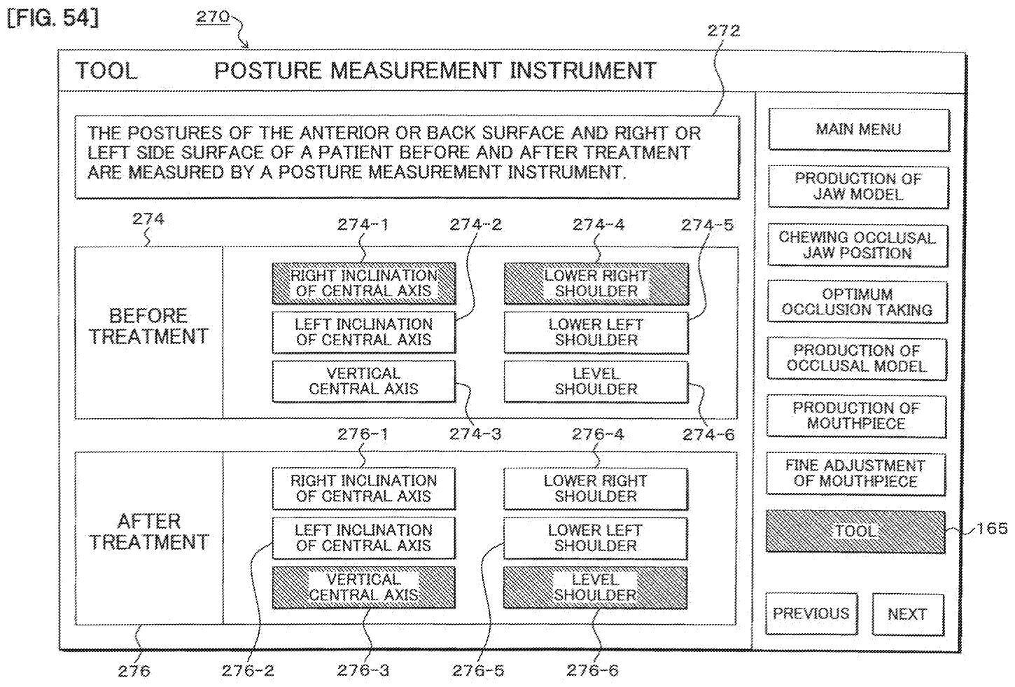

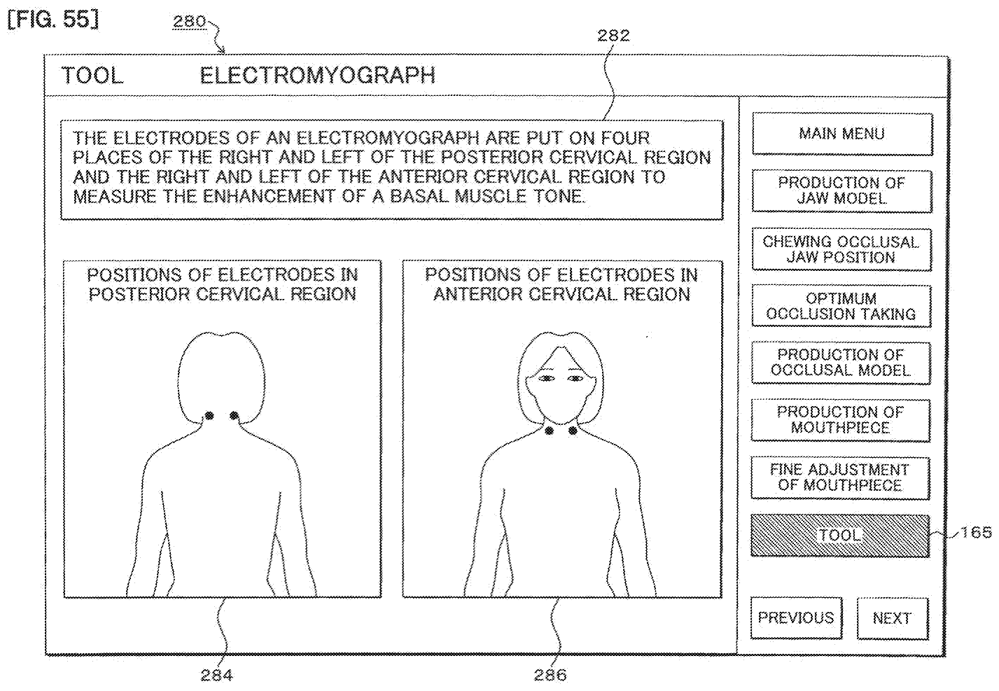

Switching to a guidance screen 270 for a posture measurement instrument illustrated in FIG. 54 is performed by touching and operating the posture measurement instrument selection button 252, and switching to a guidance screen 280 for an electromyograph illustrated in FIG. 55 is performed by touching and operating the electromyograph selection button 258.

(Guidance Screen for Posture Measurement Instrument)

As illustrated in FIG. 54, a guidance 272, a pretreatment posture input portion 274, and a posttreatment posture input portion 276 are displayed on the guidance screen 270 for a posture measurement instrument. The 72guidance 272 indicates that the postures of a patient before and after treatment are measured using a posture measurement instrument.

In the posture measurement instrument used in the present embodiment, for example, the anterior or back surface and right or left side surface of a patient are imaged by a dedicated camera and displayed on a screen in a state in which the patient is allowed to stand on a measurement stage, and the posture of the patient is evaluated.

The pretreatment posture input portion 274 is intended to input a measurement result before treatment through a touch operation and to set the result, is provided with input buttons 274-1, 274-2, and 274-3 for right inclination of a central axis through the backbone of a patient, left inclination of the central axis, and the vertical central axis, and is provided with input buttons 274-4, 274-5, and 274-6 for the lower right shoulder, the lower left shoulder, and the level shoulder with regard to the orientation of the shoulder of the patient.

The central axis of the patient depends on the lateral balance of the basal muscle tone of the posterior cervical region, and the orientation of the shoulder of the patient depends on the lateral balance of the basal muscle tone of the anterior cervical region. For example, as illustrated in the guidance image 176 of the guidance screen 170 in FIG. 43, when the enhancement of the basal muscle tone occurs in the right of the posterior cervical region, a measurement result of “Right Inclination of Central Axis” is obtained, and a set color is changed, as indicated by the oblique lines, by touching and operating the input button 274-1. When the enhancement of the basal muscle tone occurs in the left of the anterior cervical region, a measurement result of “Lower Right Shoulder” is obtained, and a set color is changed, as indicated by the oblique lines, by touching and operating the input button 274-4.

The result of measurement of the posture of the patient by the posture measurement instrument in a state in which the mouthpiece produced according to the guidance of the system for supporting production of a mouthpiece in FIG. 39 is mounted on the maxillary teeth dentition of the patient is input into the post treatment posture input portion 276.

In the measurement result obtained by the posture measurement instrument after the treatment, the lateral balance of the basal muscle tone of the posterior cervical region is achieved by mounting the mouthpiece, a measurement result of “Vertical Central Axis” is therefore obtained, and a set color is changed, as indicated by the oblique lines, by touching and operating the input button 276-3.

The lateral balance of the basal muscle tone of the cervical region is achieved by mounting the mouthpiece, a measurement result of “Level Shoulder” is therefore obtained, and a set color is changed, as indicated by the oblique lines, by touching and operating the input button 276-6.

The results of the measurement of the postures before and after the treatment, input into the screen in such a manner, is stored as treatment data in a memory, and can be read out and displayed, as needed.

Further, the measurement evaluation of the posture of the patient by the posture measurement instrument is performed regularly, for example, once every three months, in the use of the mouthpiece, and the mouthpiece is finely adjusted according to the guidance in the case of a deviation from a proper posture.

(Guidance Screen for Electromyograph)

As illustrated in FIG. 55, a guidance 282 and guidance images 284 and 286 are displayed on the guidance screen 280 for an electromyograph. The electromyograph measures the enhancement of the basal muscle tones of the posterior and anterior cervical regions. The enhancement is used for confirming the result of diagnosis by palpation. On the basis of the confirmation, the diagnosis result is input by a screen operation through the guidance screens 170 and 230 illustrated in FIG. 43 or FIG. 50.

The guidance 282 indicates putting of the electrodes of the electromyograph on the patient. It is desirable to put the electrodes of the electromyograph on four places of the right and left of the posterior cervical region and the right and left of the anterior cervical region of the patient, and to simultaneously measure the degrees of the basal muscle tones of the four places.

In this case, because it is important to put the electrodes on proper positions, the positions, on which the electrodes are put, of the two places of the right and left of the posterior cervical region of the patient are displayed on the guidance image 284, and the positions, on which the electrodes are put, of the two places of the right and left of the anterior cervical region of the patient are displayed on the guidance image 286. In such a case, the positions, which are displayed by the guidance images 284 and 286, and on which the electrodes of the electromyograph are put, are allowed to be in the posterior neck muscle in the posterior cervical region of the patient, and are allowed to be in the sternocleidomastoid muscle/scalenus muscle in the anterior cervical region.

### Alternative Embodiment of the Present Invention

The present invention is not limited to the embodiments described above but includes appropriate modifications without damaging the objects and advantages of the present invention. Further, the present invention is not limited to the numerical values set forth in the embodiments described above.

### DESCRIPTION OF REFERENCE NUMERALS

10: Maxillary model
12: Mandibular model
14: Maxillary teeth dentition
16: Mandibular teeth dentition
18: Patient
20: Posterior cervical region
20-1: Right of posterior cervical region
20-2: Left of posterior cervical region
22: Anterior cervical region
22-1: Right of anterior cervical region
22-2: Left of anterior cervical region
24-1, 26-1, 28-1, 30-1: Central incisor
24-2, 26-2, 28-2, 30-2: Lateral incisor
24-3, 26-3, 28-3, 30-3: Cuspid
24-4, 26-4, 28-4, 30-4: First premolar tooth
24-5, 26-5, 28-5, 30-5: Second premolar tooth
24-6, 26-6, 28-6, 30-6: First molar tooth
24-7, 26-7, 28-7, 30-7: Second molar tooth
32-1, 32-2, 34-1, 34-2, 48, 50: Wax
36: Curve of Spee
40: Horizontal reference plane
42: Occlusal reference plane
46: Mini-splint
52: Occludator
54: Stand
55: Support arm
56: Moveable arm
58: Shaft
60: Positioning pin
62: Fixation knob
64: Stopper portion
66: Spring
68: Adjustment knob
70: Optimum occlusal impression member
72, 82: Maxillary teeth dentition impression
74, 84: Lower teeth dentition impression
76: Mouthpiece
78: Mouthpiece body
80: Upper opening
90-1, 90-2, 92-1, 92-2, 94: Polished portion
96: Thickened portion
100: Control processor
102: Display
104: Keyboard
106: Mouse
108: Keypad
110: CPU
112: Bus
114: Control logic
116: ROM
118: RAM
120: Jaw model production guidance portion
122: Chewing occlusal jaw position guidance portion
124: Optimum occlusion taking guidance portion
126: Occlusal model production guidance portion
128: Mouthpiece production guidance portion
130: Mouthpiece fine adjustment guidance portion
131: Tool guidance
132: Screen storage portion
134: Mandibular angle position correction guidance portion
136: Mental region position correction guidance portion
138: Height difference correction guidance portion
140: Anteroposterior position adjustment guidance portion
142: Mandibular angle position fine adjustment guidance portion
144: Mental region position fine adjustment guidance portion
146: Anteroposterior position fine adjustment guidance portion

1. A mouthpiece that is detachably attached to a maxillary teeth dentition of a user,
wherein a teeth mark of a mandibular teeth dentition in an optimized chewing occlusal jaw position is formed on an occlusal surface of a mouthpiece body having an approximately uniform thickness so that a systemic basal muscle tone of a patient is balanced; and wherein a thickness of a lingual tooth surface of the mouthpiece body in a resting jaw position is adjusted so that the systemic basal muscle tone of the user is balanced.

2. The mouthpiece according to claim 1, wherein the mouthpiece body comprises a soft-type material.

3. The mouthpiece according to claim 1, wherein the mouthpiece body comprises a hard-type material.

4. A system for supporting production of a mouthpiece that is detachably mounted on a maxillary teeth dentition of a patient, the system comprising:
jaw model guidance means that guides production of maxillary and mandibular models on which teeth dentitions of the patient are arranged;
chewing occlusal jaw position guidance means that guides a medical care operation by which a chewing occlusal jaw position optimized for the patient is constructed;
optimum occlusion taking guidance means that guides production of an optimum occlusal impression member on which, in a state of constructing a chewing occlusal jaw position optimized for the patient according to the guidance of the chewing occlusal jaw position guidance means, occlusal teeth dentitions of the optimized chewing occlusal jaw position are impressed;
occlusal model production guidance means that fixes the optimum occlusal impression member produced according to the guidance of the optimum occlusion taking guidance means to an occludator in a state of sandwiching the optimum occlusal impression member between the teeth dentitions of the maxillary model and the mandibular model to guide an operation of constructing an optimized chewing occlusal model;
mouthpiece production guidance means that guides production of a mouthpiece on which a teeth dentition impression of the maxillary model and a teeth dentition impression of the mandibular model are formed by the occludator in which the chewing occlusal model is constructed, so that the optimized chewing occlusal jaw position is achieved, and which has an approximately uniform thickness; and
mouthpiece fine adjustment guidance means that guides a medical care operation of finely adjusting a predetermined lingual tooth surface of the mouthpiece so that a systemic basal muscle tone of the patient is balanced to obtain an optimum resting jaw position in a resting jaw position state of the patient on which the mouthpiece produced by the mouthpiece production guidance means is mounted.

5. The system for supporting production of a mouthpiece according to claim 4, wherein the chewing occlusal jaw position guidance means comprises:
mandibular angle position correction guidance means that guides a medical care operation of correcting a lateral position of a mandibular angle at an optimized position using a tongue/jaw position reflex of the patient, based on hypertonia of a posterior cervical region of the patient, input by a screen operation through a display;
mental region position correction guidance means that guides a medical care operation of correcting a lateral position of a mental region at an optimized position using the tongue/jaw position reflex of the patient, based on hypertonia of an anterior cervical region of the patient, input by a screen operation through the display;
height difference correction guidance means that guides a medical care operation of correcting a difference between heights of the mandibular angle and the mental region using a masseter/jaw position reflex of the patient in a state in which corrections according to the guidance of the mandibular angle position correction guidance means and the guidance of the mental region position correction guidance means have been finished; and
anteroposterior position adjustment guidance means that guides a medical care operation of adjusting an anteroposterior position of a mandibular position at an optimized position in a state in which a correction according to the guidance of the height difference correction guidance means has been finished.

6. The system for supporting production of a mouthpiece according to claim 5, wherein the mandibular angle position correction guidance means:
guides a medical care operation of putting wax on a lingual tooth surface of a mandibular right molar tooth and adjusting a thickness of the wax so that laterality of a basal muscle tone of a posterior cervical region is eliminated, when enhancement of a basal muscle tone of a right of the posterior cervical region is input by a screen operation through the display;
guides a medical care operation of putting wax on a lingual tooth surface of a mandibular left molar tooth and adjusting a thickness of the wax so that the laterality of the basal muscle tone of the posterior cervical region is eliminated, when enhancement of a basal muscle tone of a left of the posterior cervical region is input by a screen operation through the display; and
guides a medical care operation of preventing wax from being put on a lingual tooth surface of a mandibular molar tooth when no enhancement of the basal muscle tone of the posterior cervical region is input by a screen operation through the display and there is no laterality.

7. The system for supporting production of a mouthpiece according to claim 5, wherein the mental region position correction guidance means:
guides a medical care operation of putting wax on a lingual tooth surface of a mandibular right premolar tooth and adjusting a thickness of the wax so that laterality of a basal muscle tone of an anterior cervical region is eliminated, when enhancement of a basal muscle tone of a right of the anterior cervical region is input by a screen operation through the display;
guides a medical care operation of putting wax on a lingual tooth surface of a mandibular left premolar tooth and adjusting a thickness of the wax so that the laterality of the basal muscle tone of the anterior cervical region is eliminated, when enhancement of a basal muscle tone of a left of the anterior cervical region is input by a screen operation through the display; and
guides a medical care operation of preventing wax from being put on a lingual tooth surface of a mandibular premolar tooth when no enhancement of the basal muscle tone of the anterior cervical region is input by a screen operation through the display and there is no laterality.

8. The system for supporting production of a mouthpiece according to claim 6, wherein
the height difference correction guidance means guides a medical care operation of putting wax on occlusal surfaces of both maxillary first molar teeth to configure a chewing jaw position, measuring a forced expiratory residual volume with a spirometer after induction of a masseter/jaw position reflex, and correcting an elevation angle of an occlusal reference plane of a mandibular teeth dentition with respect to a horizontal reference plane so that the forced expiratory residual volume is not more than a predetermined value.

9. The system for supporting production of a mouthpiece according to claim 8, wherein the height difference correction guidance means guides a medical care operation of correcting the elevation angle so that the forced expiratory residual volume is 500 milliliters or less.

10. The system for supporting production of a mouthpiece according to claim 8, wherein the height difference correction guidance means guides a medical care operation of preparing a mini-splint in which a predetermined, corrected elevation angle of the occlusal reference plane of the mandibular teeth dentition with respect to the horizontal reference plane is set, and mounting the mini-splint on the mandibular teeth dentition to measure the forced expiratory residual volume with the spirometer.

11. The system for supporting production of a mouthpiece according to claim 10, wherein the height difference correction guidance means guides a medical care operation of preparing a plurality of kinds of mini-splints of which the corrected elevation angles are different, selecting a mini-splint having a predetermined elevation angle-corrected angle from the mini-splints, and using the mini-splint.

12. The system for supporting production of a mouthpiece according to claim 5, wherein the anteroposterior position adjustment guidance means guides a medical care operation of finely adjusting an anteroposterior position of a chewing occlusal jaw position so that a head position of the patient in rest and relaxation is maintained at a midline position with respect to a visual line of the patient.

13. The system for supporting production of a mouthpiece according to claim 12, wherein the anteroposterior position adjustment guidance means guides a medical care operation of moving forward a mandibular position when the head position of the patient in the rest and relaxation is a forward bending position and moving backward the mandibular position when the head position is a backward bending position.

14. The system for supporting production of a mouthpiece according to claim 13, wherein
in a case in which the patient is incapable of single-handedly finely adjusting an anteroposterior position, the anteroposterior position adjustment guidance means
guides a medical care operation of putting wax on a lingual tooth surface of a mandibular anterior teeth portion to adjust a thickness of the wax so that forward movement is assisted when the head position of the patient in the rest and relaxation is a forward bending position, and
guides a medical care operation of putting wax on a lingual tooth surface of a maxillary anterior teeth portion to adjust a thickness of the wax so that backward movement is assisted when the head position of the patient in the rest and relaxation is a backward bending position.

15. The system for supporting production of a mouthpiece according to claim 4, wherein the mouthpiece production guidance means:
guides production of a mouthpiece part on which the maxillary teeth dentition is carved based on the maxillary model, and which has an approximately uniform thickness;
then guides an operation of sandwiching the optimum occlusal impression member between the jaw model and the mandibular model to fix the jaw model and the mandibular model to an occludator to reproduce the optimized chewing occlusal jaw position; and
then guides an operation of mounting the mouthpiece part on the maxillary model of the occludator from which the optimum occlusal impression member is removed, and applying a teeth dentition impression of the mandibular model to an occlusal surface of the mouthpiece part based on occlusion with the mandibular model via the mouthpiece part to complete a mouthpiece.

16. The system for supporting production of a mouthpiece according to claim 4, wherein
the mouthpiece fine adjustment guidance means,
in a state of a resting jaw position of the patient in which the completed mouthpiece is mounted on a maxillary teeth dentition of the patient,
guides a fine adjustment operation of polishing a thickness of the mouthpiece located on a lingual tooth surface of a maxillary right molar tooth to reduce the thickness of the mouthpiece so that laterality of a basal muscle tone of a posterior cervical region is eliminated when enhancement of a basal muscle tone of a right of the posterior cervical region is input by a screen operation through a display,
guides a fine adjustment operation of polishing a thickness of the mouthpiece located on a lingual tooth surface of a maxillary left molar tooth to reduce the thickness of the mouthpiece so that the laterality of the basal muscle tone of the posterior cervical region is eliminated when enhancement of a basal muscle tone of a left of the posterior cervical region is input by a screen operation through the display, and
guides a fine adjustment operation of performing no fine adjustment by polishing of the mouthpiece when no enhancement of the basal muscle tone of the posterior cervical region is input by a screen operation through the display and there is no laterality.

17. The system for supporting production of a mouthpiece according to claim 4, wherein
the mouthpiece fine adjustment guidance means,
in a state of a resting jaw position of the patient in which the completed mouthpiece is mounted on a maxillary teeth dentition of the patient,
guides a fine adjustment operation of polishing a thickness of the mouthpiece located on a lingual tooth surface of a maxillary right premolar tooth to reduce the thickness of the mouthpiece so that laterality of a basal muscle tone of an anterior cervical region is eliminated when enhancement of a basal muscle tone of a right of the anterior cervical region is input by a screen operation through a display,
guides a fine adjustment operation of polishing a thickness of the mouthpiece located on a lingual tooth surface of a maxillary left premolar tooth to reduce the thickness of the mouthpiece so that the laterality of the basal muscle tone of the anterior cervical region is eliminated when enhancement of a basal muscle tone of a left of the anterior cervical region is input by a screen operation through the display, and
guides a fine adjustment operation of performing no fine adjustment by polishing of the mouthpiece when no enhancement of the basal muscle tone of the anterior cervical region is input by a screen operation through the display and there is no laterality.

18. The system for supporting production of a mouthpiece according to claim 4, wherein
the mouthpiece fine adjustment guidance means
in a resting jaw position state of the patient in which the completed mouthpiece is mounted on a maxillary teeth dentition of the patient,
guides a fine adjustment operation of polishing a thickness of the mouthpiece located on a lingual tooth surface of a maxillary anterior teeth portion to reduce the thickness of the mouthpiece so that forward movement of a mandible is assisted when an input in that a head position of the patient in rest and relaxation is a forward bending position is performed by a screen operation through the display, and
guides a fine adjustment operation of increasing the thickness of the mouthpiece located on the lingual tooth surface of the maxillary anterior teeth portion to increase the thickness of the mouthpiece so that backward movement of the mandible is assisted when an input in that the head position of the patient in the rest and relaxation is a backward bending position is performed by a screen operation through the display.

19. The system for supporting production of a mouthpiece according to claim 16, wherein the mouthpiece fine adjustment guidance means guides a fine adjustment operation of changing the thickness of the mouthpiece in a unit of micron order.

20. The system for supporting production of a mouthpiece according to claim 4, further comprising:
tool guidance means that guides a predetermined tool used for a treatment operation for producing the mouthpiece.

21. The system for supporting production of a mouthpiece according to claim 20, wherein the tool guidance means guides: a posture measurement instrument that measures a posture of a patient; and an electromyograph that measures enhancement of basal muscle tones of posterior and anterior cervical regions of the patient.

22. A mini-splint used in construction of an optimized chewing occlusal jaw position that balances a systemic basal muscle tone of a patient, the mini-splint comprising:
a fixation portion that is detachably fixedly mounted between at least a second premolar tooth and a first molar tooth in a mandibular teeth dentition of the patient; and
an angle-corrected surface that is disposed integrally with the fixation portion and fitted to an occlusal surface of the first molar tooth to set a predetermined, corrected elevation angle with respect to a horizontal reference plane of an occlusal reference plane of a mandibular teeth dentition with respect to a predetermined buccal cusp of the first molar tooth,
wherein the corrected elevation angle is a predetermined angle that allows a forced expiratory residual volume to be decreased in a state of attachment to the mandibular teeth dentition.

23. The mini-splint according to claim 22, wherein a plurality of kinds of mini-splints of which the corrected elevation angles due to the angle-corrected surface are different are prepared, and a mini-splint having a predetermined corrected elevation angle is selected from the mini-splints and fixedly mounted between at least a second premolar tooth and a first molar tooth in a mandibular teeth dentition of the patient through the fixation portion.

24. A method for producing a mouthpiece that is detachably mounted on a maxillary teeth dentition of a patient to configure an optimized chewing occlusal jaw position that balances a systemic basal muscle tone of the patient, the method comprising:
a maxillary and mandibular models production procedure of producing a maxillary model and a mandibular model on which teeth dentitions of the patient are arranged;
a mandibular angle position correction procedure of correcting a lateral position of a mandibular angle at an optimized position using a tongue/jaw position reflex of the patient;
a mental region position correction procedure of correcting a lateral position of a mental region at an optimized position using the tongue/jaw position reflex of the patient;
a height difference correction procedure of correcting a difference between heights of the mandibular angle and the mental region using a masseter/jaw position reflex of the patient in a state in which corrections according to the mandibular angle position correction procedure and the mental region position correction procedure have been finished;
an anteroposterior position adjustment procedure of adjusting an anteroposterior position of a mandibular position to an optimized position in a state in which a correction according to the height difference correction procedure has been finished;
an optimum occlusion taking procedure of producing an optimum occlusal impression member on which occlusal teeth dentitions of the optimized chewing occlusal jaw position are impressed in a state in which a chewing occlusal jaw position optimized for the patient is constructed by the anteroposterior position adjustment procedure;
an occlusal model production procedure of constructing an optimized chewing occlusal model by fixing the optimum occlusal impression member produced in the optimum occlusion taking procedure to an occludator in a state in which the optimum occlusal impression member is sandwiched between teeth dentitions of the maxillary model and the mandibular model;
a mouthpiece production procedure of producing a mouthpiece on which a teeth dentition impression of a maxillary tooth shape and a teeth dentition impression of a mandibular tooth shape are formed, so that an optimized chewing occlusal jaw position is achieved, by the occludator in which the chewing occlusal model is constructed, and which has an approximately uniform thickness; and
a mouthpiece fine adjustment procedure of performing fine adjustment so that a systemic basal muscle tone of the patient is balanced to obtain an optimum resting jaw position in a resting jaw position state of the patient on which the mouthpiece produced in the mouthpiece production procedure is mounted.

25. The method for producing a mouthpiece according to claim 24, wherein in the mandibular angle position correction procedure:
in occurrence of enhancement of a basal muscle tone in a right of a posterior cervical region, wax is put on a lingual tooth surface of a mandibular right molar tooth to adjust a thickness of the wax so that laterality of a basal muscle tone of the posterior cervical region is eliminated;
in occurrence of enhancement of a basal muscle tone in a left of the posterior cervical region, wax is put on a lingual tooth surface of a mandibular left molar tooth to adjust a thickness of the wax so that the laterality of the basal muscle tone of the posterior cervical region is eliminated; and
in absence of the laterality of the basal muscle tone of the posterior cervical region, the wax is not put on a lingual tooth surface of a mandibular molar tooth.

26. The method for producing a mouthpiece according to claim 24, wherein in the mental region position correction procedure:
in occurrence of enhancement of a basal muscle tone in a right of an anterior cervical region, wax is put on a lingual tooth surface of a mandibular right premolar tooth to adjust a thickness of the wax so that laterality of a basal muscle tone of the anterior cervical region is eliminated;
in occurrence of enhancement of a basal muscle tone in a left of the anterior cervical region, wax is put on a lingual tooth surface of a mandibular left premolar tooth to adjust a thickness of the wax so that the laterality of the basal muscle tone of the anterior cervical region is eliminated; and
in absence of the laterality of the basal muscle tone of the anterior cervical region, wax is not put on a lingual tooth surface of a mandibular premolar tooth.

27. The method for producing a mouthpiece according to claim 24, wherein
in the height difference correction procedure, wax is put on occlusal surfaces of both maxillary first molar teeth to configure a chewing jaw position, a forced expiratory residual volume is measured with a spirometer after induction of a masseter/jaw position reflex, and an elevation angle of an occlusal reference plane of a mandibular teeth dentition with respect to a horizontal reference plane is corrected so that the forced expiratory residual volume is not more than a predetermined value.

28. The method for producing a mouthpiece according to claim 27, wherein the elevation angle is corrected so that the forced expiratory residual volume is 500 milliliters or less.

29. The method for producing a mouthpiece according to claim 28, wherein a mini-splint in which a predetermined, corrected elevation angle of an occlusal reference plane of the mandibular teeth dentition with respect to a horizontal reference plane is set is prepared, and the mini-splint is mounted on the mandibular teeth dentition to measure the forced expiratory residual volume with the spirometer.

30. The method for producing a mouthpiece according to claim 29, wherein the mini-splint comprises:
a fixation portion that is detachably fixedly mounted between at least a second premolar tooth and a first molar tooth of a mandible; and
an angle-corrected surface that is formed on an occlusal surface of the first molar tooth to set a predetermined elevation angle-corrected angle with respect to a predetermined buccal cusp of the first molar tooth.

31. The method for producing a mouthpiece according to claim 29, wherein in the mini-splint, a plurality of kinds of mini-splints of which the elevation angle-corrected angles are different are prepared, and a mini-splint having a predetermined elevation angle-corrected angle is selected from the mini-splints and used.

32. The method for producing a mouthpiece according to claim 24, wherein in the anteroposterior position adjustment procedure,
an anteroposterior position of a chewing occlusal jaw position is finely adjusted so that a head position of the patient in rest and relaxation is maintained at a midline position with respect to a visual line of the patient.

33. The method for producing a mouthpiece according to claim 32, wherein in the anteroposterior position adjustment procedure, a mandibular position is moved forward when the head position of the patient in the rest and relaxation is a forward bending position, and the mandibular position is moved backward when the head position is a backward bending position.

34. The method for producing a mouthpiece according to claim 33, wherein
in the anteroposterior position adjustment procedure, in a case in which the patient is incapable of single-handedly finely adjusting an anteroposterior position,
wax is put on a lingual tooth surface of a mandibular anterior teeth portion to adjust a thickness of the wax so that forward movement is assisted when the head position of the patient in the rest and relaxation is a forward bending position, and
wax is put on a lingual tooth surface of a maxillary anterior teeth portion to adjust a thickness of the wax so that backward movement is assisted when the head position is a backward bending position.

35. The method for producing a mouthpiece according to claim 24, wherein the mouthpiece production procedure comprises:
a procedure of producing a mouthpiece part on which a maxillary teeth dentition is carved based on a produced maxillary model of a patient, and which has an approximately uniform thickness;
a procedure of, based on a produced optimum occlusal impression member which balances a systemic basal muscle tone of the patient, and on which impressions of a maxillary teeth dentition and a mandibular teeth dentition in an optimum chewing occlusal jaw position are carved, fixing the produced maxillary and mandibular models of the patient to an occludator to reproduce the optimum chewing occlusal jaw position;
a procedure of mounting the mouthpiece part on the maxillary model fixed to the occludator and applying a teeth dentition impression of the mandibular model to an occlusal surface of the mouthpiece part based on occlusion with the mandibular model via the mouthpiece part to complete a mouthpiece; and
a procedure of finely adjusting a thickness of the mouthpiece located on a lingual tooth surface of the completed mouthpiece so that an optimum resting occlusal jaw position is obtained when the completed mouthpiece is mounted on the patient.

36. The method for producing a mouthpiece according to claim 24, wherein
in the mouthpiece fine adjustment procedure, in a state of a resting jaw position of the patient on which the completed mouthpiece is mounted,
a thickness of the mouthpiece located on a lingual tooth surface of a maxillary right molar tooth is polished to reduce the thickness of the mouthpiece so that laterality of a basal muscle tone of a posterior cervical region is eliminated in occurrence of enhancement of a basal muscle tone of a right of the posterior cervical region,
a thickness of the mouthpiece located on a lingual tooth surface of a maxillary left molar tooth is polished to reduce the thickness of the mouthpiece so that the laterality of the basal muscle tone of the posterior cervical region is eliminated in occurrence of enhancement of a basal muscle tone of a left of the posterior cervical region, and
no fine adjustment by polishing of the mouthpiece is performed in absence of the laterality of the basal muscle tone of the posterior cervical region.

37. The method for producing a mouthpiece according to claim 24, wherein
in the mouthpiece fine adjustment procedure, in a state of a resting jaw position of the patient on which the completed mouthpiece is mounted,
a thickness of the mouthpiece located on a lingual tooth surface of a maxillary right premolar tooth is polished to reduce the thickness of the mouthpiece so that laterality of a basal muscle tone of an anterior cervical region is eliminated in occurrence of enhancement of a basal muscle tone of a right of the anterior cervical region,
a thickness of the mouthpiece located on a lingual tooth surface of a maxillary left premolar tooth is polished to reduce the thickness of the mouthpiece so that the laterality of the basal muscle tone of the anterior cervical region is eliminated in occurrence of enhancement of a basal muscle tone of a left of the anterior cervical region, and
no fine adjustment by polishing of the mouthpiece is performed in absence of the laterality of the basal muscle tone of the anterior cervical region.

38. The method for producing a mouthpiece according to claim 24, wherein
in the mouthpiece fine adjustment procedure, in a resting jaw position state of the patient on which the completed mouthpiece is mounted,
a thickness of the mouthpiece located on a lingual tooth surface of a maxillary anterior teeth portion is polished to reduce the thickness of the mouthpiece so that forward movement of a mandible is assisted when a head position of the patient in rest and relaxation is a forward bending position, and
the thickness of the mouthpiece located on the lingual tooth surface of the maxillary anterior teeth portion is increased to increase the thickness of the mouthpiece so that backward movement of the mandible is assisted when the head position of the patient in the rest and relaxation is a backward bending position.

39. The method for producing a mouthpiece according to claim 36, wherein a thickness of the mouthpiece is changed in a unit of micron order.

40. The method for producing a mouthpiece according to claim 24, wherein in the mouthpiece production procedure, the mouthpiece comprising a soft-type material is produced.

41. The method for producing a mouthpiece according to claim 24, wherein in the mouthpiece production procedure, the mouthpiece comprising a hard-type material is produced.
